(19) United States
(12) Patent Application Publication
(10) Pub. No.: US 2025/0288685 A1
(43) Pub. Date: Sep. 18, 2025

(54) BIOMATERIALS FOR IMPROVING BRAIN HEALING AFTER STROKE AND METHODS OF USING SAME

(71) Applicant: Duke University, Durham, NC (US)

(72) Inventors: Tatiana Segura, Durham, NC (US); Shangjing Xin, Durham, NC (US)

(21) Appl. No.: 18/860,428

(22) PCT Filed: Apr. 27, 2023
(86) PCT No.: PCT/US2023/066315

§ 371 Date: Oct. 25, 2024

Related U.S. Application Data
(60) Provisional application No. 63/335,321, filed on Apr. 27, 2022.
(60) Provisional application No. 63/335,347, filed on Apr. 27, 2022.

(51) Int. Cl. A61K 47/61; A61P 25/00; C12N 5/0783; C12N 5/079

(52) U.S. Cl. CPC A61K 47/61; A61P 25/00; C12N 5/0622; C12N 5/0636; C12N 2501/20; C12N 2501/2301; C12N 2501/25

(57) ABSTRACT

Embodiments of the instant disclosure relate to biomaterial for delivering extracellular vesicles derived from reactive cell populations for improving brain healing after injury. Also provided are methods of improving angiogenesis, axonogenesis, or inducing tissue repair using the disclosed biomaterials. Also provided are methods of making the biomaterials.

### CROSS REFERENCE TO RELATED APPLICATIONS

This application claims priority to U.S. Provisional Patent Application No. 63/335,321, filed Apr. 27, 2022, and U.S. Provisional Patent Application No. 63/335,347, filed Apr. 27, 2022, the contents of each are hereby incorporated by reference in their entirety.

### FEDERAL FUNDING LEGEND

This invention was made with Government support under Federal Grant No. 5R01NS112940 awarded by the National Institute of Neurological Disorders and Stroke (NIH/NINDS). The Federal Government has certain rights to this invention.

### INCORPORATION BY REFERENCE OF SEQUENCE LISTING

This application contains a sequence listing that has been submitted via PatentCenter in a computer readable format and is hereby incorporated by reference in its entirety. The computer readable file, created on Apr. 27, 2023, is named 109726-753010 (23-2068-WO) SequenceListing.xml and is about 6000 bytes in size.

### BACKGROUND OF THE INVENTION

Astrocytes are known to regulate the body responses to diseases and injuries in central nervous system (CNS). Under these circumstances, they become reactive and undergo functional changes. Reactive astrocytes can be both hindering or supporting CNS recovery, possibly due to their different reactive phenotypes. For example, scar-forming reactive astrocytes after spinal cord injury are required to facilitate axonal regrowth induced by releasing axon-specific growth factors from hydrogel depots. Glial fibrillary acidic protein (GFAP) labeled reactive astrocytes interact with new vessel sprouts after stroke and chemoablation of these astrocyte disrupts vascular repair and remodeling. On the other hand, neurodegenerative diseases induce neurotoxic astrocytes that lead to neuronal inflammation and degeneration. The context of when these cells may be helpful or harmful to CNS recovery and their underlying biological mechanisms is not completely understood, which limits the development of new astrocyte-based therapies for CNS diseases and injuries.

In vitro culture and activation of varying reactive astrocytes are invaluable tools to identify reactive astrocyte phenotypes and elucidate their biological functions. Microglia-induced neurotoxic astrocytes have recently been derived through in vitro activation of rat primary astrocytes with microglia-releasing interleukin-1 alpha (IL-1α), tumor necrosis factor alpha (TNF-α), and the classical complement component C1q. These cells show similar transcriptome profiles as lipopolysaccharide (LPS)-induced reactive astrocytes in vivo, which present in various neurodegenerative diseases and induce the death of neurons and oligodendrocytes via the secretion of saturated lipids. There is potential to develop more cytokine cocktails for induction of varying reactive astrocytes, which helps the search for new astrocyte targets or therapies to treat CNS diseases. However, direct transplantation of these reactive astrocytes can be difficult because of the possible immune rejection and limited cell sources. While patient-derived astrocyte progenitor cells have shown promising therapeutic outcomes for CNS diseases, this strategy still relies on the cell differentiation into pro-repair lineage with the knowledge of reactive astrocyte categories and functions.

What is needed are improved delivery methods for delivering active components from reactive astrocytes or other reactive cell types in order to harness the therapeutic potential of these reactive cell populations.

### BRIEF SUMMARY OF THE DISCLOSURE

The present disclosure provides, in part, an extracellular vesicle hydrogel microparticle (EV-HMP), the microparticle comprising: (i) a hydrogel microparticle (HMP) and (ii) an extracellular vesicle (EV) derived or collected from a cell population, wherein the extracellular vesicle is immobilized to the HMP.

In various aspects, the extracellular vesicle of the EV-HMP is labeled with an azido sugar and the hydrogel microparticle is functionalized with a strained alkyne. In some aspects, the extracellular vesicle (EV) is immobilized to the hydrogel microparticle (HMP) via a triazole linkage between the azido sugar and the strained alkyne.

Further aspects of the present disclosure relate to methods of making an extracellular vesicle hydrogel microparticle (EV-HMP), the methods comprising (a) isolating an extracellular vesicle labeled with an azido sugar (azido-EV) from a cell population; and (b) contacting the azido-EV with a hydrogel microparticle (HMP) functionalized with a strained alkyne so that the azido sugar of the azido-EV forms a stable triazole linkage with strained alkyne of the HMP to make the extracellular vesicle hydrogel microparticle (EV-HMP).

In various aspects, step (b) of the method of making an EV-HMP provided herein can comprise incubating the azido-EV with the hydrogel microparticle at an elevated temperature for at least 10 minutes, at least 15 minutes, at least 20 minutes, at least 25 minutes, or at least 30 minutes. In some aspects, the elevated temperature can be from about 30° C. to about 40° C.

In various aspects, the methods of making an EV-HMP as provided herein may further comprise culturing the cell population in the presence of the azido sugar before isolating the azido-EV from the population.

In any of the preceding aspects, the azido sugar can comprise-azidoacetylmannosamine-tetraacylated (Ac4MannAz) or 9-azido sialic acid, 6-Azide-Trehalose (6-TreAz), 8-Azido-3,8-dideoxy-D-manno-octulosonic acid (Kdo Azide), 9-azido-9-deoxy-N-acetylneuraminic acid (9AzNeu5Ac), N-azidoacetylglucosamine-tetraacylated (Ac4GlcNAz), N-azidoacetylgalactosamine-tetraacylated (Ac4GalNAz), 6-azido-6-deoxy-N-acetyl-glucosamine triacylated (Ac3-6AzGlcNAc), or any combination thereof. In any of the preceding aspects, the strained alkyne can comprise dibenzocyclooctyne (DBCO), bicyclo[6.1.0]non-4-yn-9-ylmethanol (BCN), azadibenzocyclooctyne (ADIBO), cyclooctyne (OCT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), 2,3,6,7-tetramethoxy-DIBO (TMDIBO), sulfonylated DIBO (S-DIBO), or carboxymethylmonobenzocyclooctyne (COMBO), pyrrolocyclooctyne (PYRROC), or any combination thereof. In any of the preceding aspects, the hydrogel microparticle can comprise hyaluronic acid poly (ethylene glycol), gelatin, alginate, collagen, Methyl cellulose, or any combination thereof.

In various aspects, the cell population of any of the EV-HMPs or methods described herein comprises astrocytes or T-cells.

In any of the foregoing compositions and methods, the cell population may be cultured in a medium comprising at least one cytokine or cytokine antibody. In some aspects, the cell population may be cultured in the medium comprising the at least one cytokine or cytokine antibody for at least 10, at least 15, at least 20, or at least 24 hours. In some aspects, the medium comprising the at least one cytokine or cytokine antibody comprises an anti-CD28 antibody, an anti-IL4 antibody, IL-2, IL-12, an anti-IFNγ antibody, IL-4, TGFβ1, IL-1α, TNF, C1q, or any combination thereof.

In various aspects, the cell population comprises astrocytes and the medium comprising the at least one cytokine or cytokine antibody comprises IL-4, IL-1α, TNF, C1q, or any combination thereof. For example, in some aspects, the medium comprising the at least one cytokine or cytokine antibody comprises (a) IL-1α, TNF, and/or C1q; or (b) IL-4 and/or C1q.

In various aspects, the cell population comprises T-cells and the medium comprising the at least one cytokine or cytokine antibody comprises anti-CD28 antibody, anti-IL4 antibody, IL-2, IL-12, anti-IFNγ antibody, IL-4, TGFB1, or any combination thereof. For example, in some aspects, the medium comprising the at least one cytokine or cytokine antibody comprises (a) anti-CD28 antibody, anti-IL4 antibody, IL-2, and/or IL-12; (b) anti-CD28 antibody, anti-IFNγ antibody, IL-2, and/or IL-4; or (c) anti-CD28 antibody, anti-IFNγ antibody, anti-IL4 antibody, and/or TGFβ1.

In any of the compositions and methods herein, the medium comprising the at least one cytokine or cytokine antibody can comprise from about 0.05 to about 3 μg/mL of the anti-CD28 antibody, about 0.5 to about 5 μg/mL of the anti-IL4 antibody, about 1 to about 10 ng/ml of the IL-2, about 5 to about 20 ng/ml of the IL-12, about 0.5 to about 5 μg/mL of the anti-IFNγ antibody, about 5 to about 20 ng/ml of the IL-4, about 0.5 to about 5 ng/ml of the TGFB1, about 1 to about 5 ng/ml of the IL-1α, about 10 to about 50 ng/ml of the TNF, about 100 to about 800 ng/ml of the C1q, or any combination thereof. For example, in some aspects, the medium comprising the at least one cytokine or cytokine antibody can comprise about 0.5 μg/mL of the anti-CD28 antibody, about 1 μg/mL of the anti-IL4 antibody, about 5 ng/ml of the IL-2, about 10 ng/ml of the IL-12, about 1 μg/mL of the anti-IFNγ antibody, about 10 ng/ml of the IL-4, about 2 ng/ml of the TGFB1, about 3 ng/mL of the IL-1α, about 30 ng/ml of the TNF, about 400 ng/ml of the C1q or any combination thereof.

In any of the compositions or methods herein, the medium comprising the at least one cytokine or cytokine antibody can further comprise an extracellular vesicle collecting media (EV collecting media). In various aspects, the EV collecting media can comprise Dulbecco's Modified Eagle Medium (DMEM), FBS, hydrocortisone, an antibacterial agent, glutamine, insulin, NaPyruvate, N-acetyl-L-cysteine or any combination thereof.

Further aspects of the present disclosure are directed to EV-HMPs prepared by any method provided herein. Also provided is an EV-HMP suspension comprising two or more EV-HMPs provided herein and/or prepared by a method provided herein.

Further aspects of the present disclosure are directed to an extracellular vesicle microporous annealed particle hydrogel (EV-MAP hydrogel), wherein the EV-MAP hydrogel comprises one or more EV-HMPs provided herein and a crosslinker, wherein at least two EV-HMPs are linked by a crosslinker. For example, in some aspects, at least 50%, at least 60%, at least 70%, at least 80%, at least 85%, or at least 90% of EV-HMPs in the hydrogel are linked by the crosslinker. In various aspects, the crosslinker can comprise two or more azide groups. In various aspects, the crosslinker can comprise tetra-arm PEG-azide.

Further aspects of the present disclosure are directed to methods of making an extracellular vesicle microporous annealed particle hydrogel (EV-MAP), the methods comprising contacting one or more EV-HMPs as provided herein with a crosslinker to obtain the EV-MAP. In various aspects, the methods can comprise contacting the one or more EV-HMPs with the crosslinker in vivo or in situ. In various aspects, the crosslinker can comprise two or more azide groups. In various aspects, the crosslinker can comprise tetra-arm PEG-azide.

Also provided are EV-MAP hydrogels prepared by any method provided herein.

Further aspects of the disclosure provide for a pharmaceutical composition comprising one or more EV-HMPs provided herein and a pharmaceutically appropriate carrier. Additional aspects of the present disclosure provide for a pharmaceutical composition comprising an EV-MAP hydrogel as prepared by any method provided herein and a pharmaceutically appropriate carrier.

Additional aspects of the disclosure provide for a kit comprising: (a) the pharmaceutical composition comprising one or more EV-HMPs as provided herein and a pharmaceutically appropriate carrier and (b) a crosslinker. In various aspects, the crosslinker can comprise two or more azide groups. In some aspects, the crosslinker comprises tetra-arm PEG-azide.

Further aspects of the present disclosure provide for methods of inducing and/or increasing angiogenesis and/or axonogenesis in a subject, the method comprising administering to the subject a therapeutically effective amount of a pharmaceutical composition as provided herein (e.g., comprising one or more EV-HMPs or an EV-MAP hydrogel) to the subject such that angiogenesis and/or axonogenesis is increased in the subject. In some aspects, the method comprises inducing and/or increasing angiogenesis and/or axonogenesis in the brain of the subject.

Further aspects of the present disclosure provide for methods of inducing tissue repair in a subject, the methods comprising administering to the subject a therapeutically effective amount of a pharmaceutical composition as provided herein (e.g., comprising one or more EV-HMPs or an EV-MAP hydrogel) to the subject such that the tissue repair in the subject is induced. In various aspects, the tissue repair can comprise brain tissue repair.

In various aspects, the subject treated by the methods herein may have or be suspected of having a brain injury. In various aspects, the brain injury can comprise or be caused by a traumatic injury (e.g., external injury), an aneurysm, a stroke, an infection, a cancer, or a combination of any thereof.

Further aspects of the present disclosure provide for methods of treating a stroke in a subject, the method comprising administering to the subject a therapeutically effective amount of a pharmaceutical composition provided herein (e.g., comprising one or more EV-HMPs or an EV-MAP hydrogel) such that the stroke is treated in the subject.

In various aspects, methods of treating a stroke provided herein can comprise reducing or preventing short-term tissue damage at the stroke site. In various aspects short-term tissue damage comprises damage to surrounding tissue at a stroke site that occurs within 24 hours of the stroke.

In various aspects, methods of treating a stroke provided herein can comprise promoting and/or inducing long-term recovery after the stroke. In further aspects, methods of treating a stroke can comprise inducing and/or increasing angiogenesis and/or axogenesis at the site of the stroke.

In any of the methods herein, the stroke may be a hemorrhagic stroke. In any of the methods herein, the stroke may be an occlusive stroke.

Any of the methods of inducing and/or increasing angiogenesis and/or axonogenesis, inducing tissue repair, or treating a stroke may comprise administering a therapeutically effective amount of a pharmaceutical composition comprising an EV-HMP suspension one or more EV-HMPs provided herein and a pharmaceutically acceptable carrier. In various aspects, these methods may further comprise administering a crosslinker to the subject. In various aspects, the crosslinker may be administered before the pharmaceutical composition. In various aspects, the crosslinker may be administered simultaneously with the pharmaceutical composition. In various aspects, the crosslinker may be administered after the pharmaceutical composition. For example, in some aspects, the crosslinker may be administered 1 to 10 days after the pharmaceutical composition. In some aspects, the crosslinker is administered about 5 days after the pharmaceutical composition. In any of these aspects, the crosslinker may comprise can comprise two or more azide groups. For example, in some methods the crosslinker comprises tetra-arm PEG-azide.

In any of the foregoing or related aspects, the pharmaceutical composition and/or crosslinker can both be delivered into the brain of the subject. In various aspects, the pharmaceutical composition and/or the crosslinker are each administered via stereotaxic injection. In various aspects, the pharmaceutical composition and/or the crosslinker are each administered intrathecally, intraventricularly, via direct infarct injection, via peri-infarct injection, or any combination thereof. In various aspects, the pharmaceutical composition and/or the crosslinker are each administered into a stroke core of the subject. In various aspects, the crosslinker is administered via the same route as the therapeutically effective amount of the pharmaceutical composition.

In any of the methods provided herein, the subject may be a mammal.

Also provided herein are methods of preparing a population of reactive astrocytes, the method comprising culturing a population of astrocytes in a culture medium comprising at least one cytokine or cytokine antibody. In various aspects, the medium comprising the at least one cytokine or cytokine antibody comprises IL-4, IL-1α, TNF, C1q or any combination thereof. For example, in some aspects, the medium comprising the at least one cytokine or cytokine antibody comprises (a) IL-1α, TNF, and/or C1q; or (b) IL-4 and/or C1q.

Further aspects of the present disclosure provide for methods of preparing a population of activated T-cells, the method comprising culturing a population of T-cells in a culture medium comprising at least one cytokine or cytokine antibody. In various aspects, the medium comprising the at least one cytokine or cytokine antibody comprises anti-CD28 antibody, anti-IL4 antibody, IL-2, IL-12, anti-IFNγ antibody, IL-4, TGFB1, or any combination thereof. For example, in some aspects, the medium comprising the at least one cytokine or cytokine antibody comprises (a) anti-CD28 antibody, anti-IL4 antibody, IL-2, and/or IL-12; (b) anti-CD28 antibody, anti-IFNγ antibody, IL-2, and/or IL-4; or (c) anti-CD28 antibody, anti-IFNγ antibody, anti-IL4 antibody, and/or TGFβ1.

Also provided herein is an extracellular vesicle hydrogel microparticle (EV-HMP), the microparticle comprising: (i) a hydrogel microparticle (HMP) functionalized with a strained alkyne and (ii) an extracellular vesicle (EV) labeled with an azido sugar derived or collected from a cell population, wherein the cell population comprises astrocytes or T-cells cultured in a medium comprising at least one of an anti-CD28 antibody, an anti-IL4 antibody, IL-2, IL-12, an anti-IFNγ antibody, IL-4, TGFB1, IL-1α, TNF, C1q, or any combination thereof, and wherein the extracellular vesicle is immobilized to the HMP via a stable triazole linkage between the strained alkyne and the azido sugar.

These and other features and advantages of the disclosure will be fully understood from the following detailed description and the accompanying drawings.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A depicts a schematic illustrating the generation of two reactive astrocytes from naïve primary astrocytes, and the use of their derived EVs to make EV-MAPs for stroke treatment;

FIG. 1B depicts a schematic of the generation of two reactive astrocyte populations from naïve primary astrocytes according to certain aspects of the present disclosure.

FIG. 2A depicts a schematic of treatments to generate neurotoxic (Ntx) and reparative (Rep) astrocytes and a heatmap of gene expression of untreated, neurotoxic and reparative astrocytes.

FIG. 2B depicts size characterization and concentrations of EVs derived from UT, NTx, and Rep astrocytes from nanoparticle tracking analysis.

FIG. 2C depicts a schematic illustrating the metabolic labeling process to produce azide-bearing astrocytic EVs and the conjugation between astrocytic EVs and strain-alkyne microgels.

FIG. 2D is a schematic and confocal image of a selection of the entire stroke infarct as region of interest for void space quantification to determine the degradation of MAPS.

FIG. 2E is representative Z-projection confocal images and quantification of stroke infarct area after injection with vehicle (μgels), MAPs alone, NTx-EV-MAP or Rep-EV-MAP. Scale bars, 500 μm.

FIG. 3A is a Venn diagram showing the overlapping genes among Neurotoxic (Ntx), Reparative (Rep) and untreated astrocytes derived according to the disclosure herein.

FIG. 3B is a Venn diagram showing that compared to untreated (UT) astrocytes, NTx astrocytes had more up-regulated genes than Rep astrocytes.

FIG. 3C is an abundance comparison among three astrocytes of functional genes showed in the heatmap of FIG. 2A.

FIG. 3D is heatmap showing the up-regulation of pan-reactive, LPS-specific, and MCAO-specific markers in our UT, NTx, and Rep astrocytes as a phenotype comparison to the astrocytes in Liddelow et al (Nature 541, 481-487 (2017).

FIG. 4A-4B depicts stitched 4X Z-projection large scan image of a stroke only sample (FIG. 4A) and quantification (FIG. 4B) of stroke volume post-stroke.

FIG. 4C-4D is a schematic depicting the grid walking setup (FIG. 4C) and quantification (FIG. 4D) of healthy (n=5 mice) and stroke only (n=6 mice) showing functional differences after stroke.

FIG. 4E-4F depicts Stitched 20X Z-projection images of large image scans and inset images of tomato lectin (green), GFAP (red), and NF200 (white) in the stroke and peri-infarct regions for sham (FIG. 4E) and EV only (FIG. 4F) groups. Scale bars (large scans), 500 μm; Scale bars (insets), 100 μm.

FIG. 4G-4H depict plots showing quantification of total axon area (FIG. 4G), and total vessel area (FIG. 4H) in the infarct between Sham (n=3 mice) and EV only (n=4 mice).

FIG. 5A is a histogram of microgel size after conjugation of AD-EVs onto hyaluronic acid microgels.

FIG. 5B depicts a confocal image of void space in MAPS infiltrated by high molecular weight dextran.

FIG. 5C is a schematic illustrating the metabolic labeling process to produce azide-bearing astrocytic EVs and the conjugation between astrocytic EVs and strain-alkyne microgels.

FIG. 5D-5E depicts Z-projection confocal images (FIG. 5D) and concentrations of EV suspensions (FIG. 5E) indicating the successful conjugation of ADEVs onto microgels via strain-promoted azide-alkyne click chemistry. Scale bars, 100 μm. Statistics were calculated by two-tailed unpaired t-test.

FIG. 5F depicts Z-projection confocal images showing the different thickness of EV layers at different EV concentrations on the surface of microgels. Scale bars, 100 μm.

FIG. 5G depicts in situ rheological monitoring of the MAPS annealing process with the presence of different concentrations of EVs on the microgels.

FIG. 6A depicts a stitched 4X Z-projection large scan image of a sample at Day 21 after stroke with MAPS injected into the infarct.

FIG. 6B depicts Z-projection 20X confocal images showing the MAPS degradation after implantation. Scale bars, 100 μm.

FIG. 6C depicts Z-projection large scan images of DAPI staining showing the gaussian blur and mask at different variances (o).

FIG. 7A depicts stitched 20X Z-projection large scan and inset images of perfused tomato lectin (red) in the stroke infarcts. Scale bars (large scans), 500 μm; Scale bars (insets), 100 μm.

FIG. 7B depicts binarization of tomato lectin signals within stroke infarct for blood vessel quantification. Scale bars, 250 μm.

FIG. 7C-7E are bar graphs depicting quantification of total blood vessel area normalized to tissue area (FIG. 7C), blood vessel infiltration (FIG. 7D), and diameter of blood vessels (FIG. 7E) showing improved angiogenesis in both NTx- and Rep-EV+MAPS groups (n=4 or 5 mice).

FIG. 7F-7H depict images (FIG. 7F) and quantification of blood vessel intensity, as measure of distance (FIG. 7G) and in average (FIG. 7H) showing Rep-EV+MAPS had more mature vessels than NTx-EV+MAPS.

FIG. 7I-7J depict Z-projection images (FIG. 7I) and quantification (FIG. 7J) showing pericyte (white) coverage over the new blood vessels (red) in the stroke infarct, indicating vessels are more mature in Rep-EV+MAPS group. (n=4 or 5 mice). Yellow lines are 5 μm boundary around the vessels as an illustration of vessel ROI for quantification of the interactions between pericytes and vessels. Scale bars, 100 μm.

FIG. 7K-7L depict representative Z-projection images (FIG. 7K) and quantification (FIG. 7L) of pericytes by colocalizating PDGFRβ (green) and NG2 (red). Scale bars, 100 μm.

FIG. 8A-8D are bar graphs depicting quantification of vessel count normalized to tissue area (FIG. 8A), branch count (FIG. 8B), length of longest vessel (FIG. 8C), and total vessel length (FIG. 8D) showing improved angiogenesis in the Rep-EV+MAP group (n=4 or 5 mice).

FIG. 8E-8F depicts representative Z-projection images (FIG. 8E) and quantification (FIG. 8F) showing pericytes (white) constituted a large population of infiltrated cells and they were not restricted to the area around newly formed blood vessels (n=4 or 5 mice). Scale bars, 500 μm.

FIG. 8G depicts a representative Z-projection image showing pericytes were mostly restrict to the blood vessel surroundings in the peri-infarct.

FIG. 8H depicts a representative 3D rendering image showing the colocalization of PDGFRβ and NG2 signals.

FIG. 8I depicts representative stitched 20X Z-projection large scan images of pericyte staining in the stroke infarct using PDGFRβ (green) and NG2 (red). Scale bars, 500 μm.

FIG. 9A-9B depicts stitched 20X Z-projection large scan and inset images (FIG. 9A) and quantification (FIG. 9B) of Iba-1 (white) in the stroke infarct (n=4 or 5 mice). Scale bars (large scans), 500 μm; Scale bars (insets), 100 μm.

FIG. 9C-9D depicts stitched 20X Z-projection large scan and inset images (FIG. 9C) and quantification (FIG. 9D) of GFAP (red) in the stroke infarct (n=4 or 5 mice). Scale bars (large scans), 500 μm; Scale bars (insets), 100 μm.

FIG. 10A depicts representative stitched 20X Z-projection large scan and inset images of NF200 (white) in the stroke and peri-infarct regions. Scale bars (large scans), 500 μm; Scale bars (insets), 100 μm.

FIG. 10B depicts a schematic showing the region of interest for the stroke infarct, peri-infarct, and the tissue area between MAPS for image quantification.

FIG. 10C-10E are bar graphs depicting quantification of total axon area in infarct normalized to tissue area (FIG. 10C), axon area in peri-infarct (FIG. 10D), and axon infiltration (FIG. 10E) showing NTx-EV+MAPS promotes axon regrowth in both infarct and peri-infarct after stroke (n=4 or 5 mice).

FIG. 10F-10H depict a schematic of testing conditions (FIG. 10F) and a plot of missed/total step ratio (FIG. 10G) and a plot of % foot faults of total steps (FIG. 10H) from Grid walking behavioral studies showing functional recoveries from the treatment of Rep-EV+MAPS after stroke (n=5 mice).

FIG. 11A is a schematic showing the process of sample preparation, data acquisition on LC-MS/MS, and differential and enrichment analysis.

FIG. 11B depicts a volcano plot showing differential expression of proteins between Rep-EVs and UT-EVs.

FIG. 11C depicts a volcano plot showing differential expression of proteins between NTx-EVs and UT-EVs.

FIG. 11D depicts a volcano plot showing differential expression of proteins between Rep-EVs and NTx-EVs.

FIG. 11E-11F depict PSEA-Quant analysis of up-regulated pathways using abundance ratios of Rep/NTx (FIG. 11E) and NTx/Rep (FIG. 11F).

FIG. 11G depicts a heatmap of core proteins from PSEA-Quant analysis showing the differential expression levels of these key proteins among UT, NTx- and Rep-EVs.

FIG. 12A is a Venn diagram showing the overlapping proteins among UT, NTx, and Rep astrocytes.

FIG. 12B is a volcano plot with significance ratio of 1.3 and fold change of 1 showing differential expression of proteins between NTx-EVs and UT EVs.

FIG. 12C is a volcano plot with significance ratio of 1.3 and fold change of 1 showing differential expression of proteins between UT-EVs and Rep-EVs.

FIG. 12D is a volcano plot with significance ratio of 1.3 and fold change of 1 showing differential expression of proteins between Rep-EVs and Ntx-EVs

FIG. 12E is a plot of the difference of log 2 fold change of C3 comparing NTx to UT between astrocyte RNA sequencing and EV proteomics data, showing inflammatory proteins were less encapsulated within EVs by astrocytes.

FIG. 13A depicts stitched 20X Z-projection large scan and inset images of NF200 (white) in the stroke and peri-infarct regions, showing all Th1, Th2, Treg-EV+MAPS groups improve axon regrowth at Day 21 after stroke.

FIG. 13B depicts stitched 20X Z-projection large scan and inset images of tomato lectin (white) in the stroke and peri-infarct regions, showing all Th1, Th2, Treg-EV+MAPS groups do not improve angiogenesis at Day 21 after stroke.

FIG. 13C-13D provides bar graphs quantifying total axon area (FIG. 13C), and total vessel area (FIG. 13D) in the infarct among MAPS and Th EV+MAPS groups.

### DETAILED DESCRIPTION

The present disclosure is based, in part, on the discovery by the inventors that the use of astrocyte-derived extracellular vesicles (ADEVs) and/or T-cell derived extracellular vesicles present therapeutic benefits on brain tissue repair after stroke. EVs are lipid bilayer nanoparticles containing proteins, RNAs, and metabolites in response to cell states. They participate in cell-cell communication to orchestrate biological outcomes. For example, astrocytic EVs released in response to IL-1β and TNFα reduce dendritic growth of targeted neurons, while EVs secreted from untreated astrocytes enhance neuronal survival and electrophysiological function. The inventors showed that ADEVs isolated from naïve or activated astrocytes delivered by microporous annealed particle (MAP) scaffolds in a mouse photothrombotic stroke model showed astrocyte-coordinated tissue repair, thus providing for a novel ADEV-MAP based therapy for treating stroke. Further, the inventors showed that extracellular vesicles obtained from other treated cells (e.g., T-cells) can also promote brain repair after stroke-thereby adding TDEV-MAP based therapies as additional stroke treatment methods.

Accordingly, various aspects of the present disclosure are directed to extracellular vesicles isolated from treated astrocytes and/or T-cells, EVs immobilized on hydrogel microparticles (EV-HMPs) and microgels comprising them. Further aspects of the disclosure relate to methods of using these EV-HMPs and microgels in methods to induce brain tissue repair, improve angiogenesis, and/or promote or induce recovery after a stroke.

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition (e.g., a stroke) manifested by a patient or to which a patient may be susceptible. The aim of treatment includes the alleviation or prevention of symptoms, slowing or stopping the progression or worsening of a disease, disorder, or condition and/or the remission of the disease, disorder or condition (e.g., a stroke).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, the terms “prevent,” “preventing,” “prevention,” “prophylactic treatment” and the like refer to reducing the probability of developing a disease, disorder or condition (e.g., stroke) in a subject, who does not have, but is at risk of or susceptible to developing a disease, disorder or condition. The term “effective amount” or “therapeutically effective amount” refers to an amount sufficient to effect beneficial or desirable biological and/or clinical results. In other words, in an aspect, preventing stroke, brain damage (including damage to neurons, axons, or glial) is intended. The words “prevent” and “preventing” and “prevention” also refer to prophylactic or preventative measures for protecting or precluding a subject (e.g., an individual) not having brain damage from progressing to that complication (e.g., after suffering a stroke).

As used herein, the term “stroke core” refers to an area immediately surrounding a site of occlusion or hemorrhage in a stroke.

As used herein, the term “treating a stroke” can refer to inducing a positive or beneficial outcome to a patient after suffering a stroke. This can occur by, for example, minimizing, reducing, or preventing tissue damage (i.e., brain tissue damage, neuronal tissue damage or epithelial cell damage) in the stroke core or in a nearby region of the brain. Treating a stroke can involve reducing short term tissue damage (i.e., at the site of a stroke, at the stroke core). As used herein, “short term” tissue damage refers to damage that occurs within the first 24 hours after the stroke begins. Treating a stroke can also involve promoting and/or inducing long term recovery after the stroke. Long term recovery can comprise, for example, regaining neural function lost at the time of the stroke (e.g., motor function, somatosensory function, cognitive function etc). It can also comprise repairing tissue damage to the brain that occurs after the stroke (e.g., at the stroke core but also at distal regions in the brain). Treating a stroke also explicitly covers reducing total infarct area.

As used herein, the term “stroke” refers to an incidence where a blood vessel to or in a brain is occluded (blocked) or hemorrhages. Any “stroke” described herein may be a hemorrhagic or occlusive stroke. In various aspects, a stroke may be a hemorrhagic stroke which refers to a stroke caused by bleeding into the brain by a rupture of a blood vessel. Hemorrhagic strokes may be further subdivided into intracerebral hemorrhage (ICH), which is bleeding into the brain parenchyma, and subarachnoid hemorrhage (SAH), which is bleeding into the subarachnoid space. In various aspects, a stroke may be an occlusive stroke, also known as an ischemic stroke, which refers to a stroke where a blood vessel supplying blood to the brain is obstructed.

As used herein, the term “angiogenesis” refers to an increase in blood vessel flow into a region of interest and can comprise, for example, migration, growth, and differentiation of endothelial cells, which line the inside wall of blood vessels, formation of new blood vessels, or any combination thereof.

As used herein, the term “axonogenesis” refers to a de novo generation of a long process of a neuron, including the terminal branched region. It can also refer to the morphogenesis or creation of shape or form of the developing axon, which carries efferent (outgoing) action potentials from the cell body towards target cells.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “extracellular vesicle” refers to a lipid bilayer nanoparticle derives from a cell in response to a cell state.

As used herein, the term “astrocyte-derived extracellular vesicle” (or ADEV) refer to those lipid bilayer nanoparticles derived from astrocytes in response to cell states, the ADEV may contain, but are not limited to, proteins, RNAs, and metabolites.

As used herein, the term “T-cell-derived extracellular vesicle” (or TDEV) refer to those lipid bilayer nanoparticles derived from T-cells in response to cell states that include, but are not limited to, proteins, RNAs, and metabolites.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample can be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It can be caused by an external factor, such as an infectious disease, or by internal dysfunctions, such as stroke, infarction, and the like.

“Contacting” as used herein, e.g., as in “contacting a sample” refers to contacting a sample directly or indirectly in vitro, ex vivo, or in vivo (i.e., within a subject as defined herein). Contacting a sample can include addition of a compound (e.g., a nucleic acid and/or vector as provided herein) to a sample, or administration to a subject. Contacting encompasses administration to a solution, cell, tissue, mammal, subject, patient, or human. Further, contacting a cell includes adding an agent to a cell culture.

As used herein, the term “therapeutic agent” means an agent utilized to treat, combat, ameliorate, prevent or improve an unwanted condition or disease of a subject, such as stroke and associated complications.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Nonhuman animals may further comprise companion animals, livestock animals or laboratory animals. Companion animals of the disclosure can include any animal domesticated by humans for company or entertainment (e.g., dog, cat, horse, rabbit). Livestock refers to any animal kept by humans for agricultural or dietary purposes (e.g., cow, sheep, chickens, pigs, etc). Laboratory animals refer to animals used by humans in a laboratory for experimental purposes (e., mice or rats). The categories provided herein are not mutually exclusive and it is envisioned that some animals (subjects herein) may be part of more than one category (e.g., dogs can be both companion and animals and laboratory animals; pigs can be both livestock and laboratory animals). The methods and compositions disclosed herein can be used on a sample either in vitro (for example, on isolated cells or tissues) or in vivo in a subject (i.e., living organism, such as a patient).

As used herein, the term “sequence identity” refers to the number of identical or similar residues (i.e., nucleotide bases or amino acid) on a comparison between a test and reference nucleotide or amino acid sequence. Sequence identity can be determined by sequence alignment of nucleic acid to identify regions of similarity or identity. As described herein, sequence identity is generally determined by alignment to identify identical residues. Matches, mismatches, and gaps can be identified between compared sequences. Alternatively, sequence identity can be determined without taking into account gaps as the number of identical positions/length of the total aligned sequence x 100. In one non-limiting embodiment, the term “at least 90% sequence identity to” refers to percent identities from 90 to 100%, relative to the reference nucleotide or amino acid sequence. Identity at a level of 90% or more is indicative of the fact that, assuming for exemplary purposes a test and reference oligonucleotide or length of 100 nucleotides are compared, no more than 10% (i.e., 10 out of 100) of the nucleotides in the test oligonucleotide differ from those of the reference oligonucleotide. Differences are defined as nucleic acid or amino acid substitutions, insertions, or deletions.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “cytokine” refers to a small molecule, protein or peptide released by a cell (e.g., an immune cell) that can control growth and activity of other cells (e.g., other cells in the immune system).

As used herein, a “cytokine antibody” refers to an antibody or antigen binding fragment thereof that selectively binds a cytokine or its receptor. In some embodiments, a cytokine antibody disclosed herein can be a full-length antibody or an antigen-binding fragment thereof. In some aspects, the cytokine antibody may be a neutralizing antibody towards its target cytokine. In some embodiments, a cytokine antibody disclosed herein can be a full-length antibody, such as an IgG molecule. In some embodiments a cytokine antibody disclosed herein can be an antibody fragment and can be a Fab, a (Fab′)2, and/or a single-chain antibody. In some embodiments, a cytokine antibody disclosed herein can be a human antibody or a humanized antibody.

Unless otherwise defined, all technical terms used herein have the same meaning as commonly understood by one of ordinary skill in the art to which this disclosure belongs.

### II. Compositions

In various aspects, the present disclosure is directed to compositions comprising extracellular vesicles isolated from treated astrocytes and/or T-cells, EVs immobilized on hydrogel microparticles (EV-HMPs) and microgels comprising them. Each of these components is described in more detail below.

(a) Extracellular Vesicles Derived from Cell Populations

### Cell Populations

As noted, aspects of the present disclosure are directed to compositions containing extracellular vesicles derived from treated cell populations. In various aspects, these treated cell populations are identified as “reactive” populations, to distinguish from non-reactive untreated cell populations. In some aspects, the treated cell populations are referred to herein as “activated”, to distinguish from untreated “quiescent” cell populations. There are many possible cell populations that can are generate extracellular vesicles usable in the compostions and methods herein. Exemplary cell populations include astrocytes and T-cells. Further examples of these populations include “neurotoxic” or “reparative” astrocytes and regulatory T-cells or activated helper T cells (e.g, Th1 or Th2 cell populations). These populations are described further below.

In various aspects, cell population used to derive extracellular vesicles may be cultured in a medium comprising one or more cytokines or cytokine antibodies. In various aspects, the one or more cytokines may comprise a protein, antibody, or small molecule. For example, in some aspects, the one or more cytokines or cytokine antibodies may include an anti-CD28 antibody, an anti-IL4 antibody, IL-2, IL-12, an anti-IFNγ antibody, IL-4, TGFβ1, IL-1α, TNF, C1q, or any combination thereof. Each of these components are described in more detail below. CD28 and anti-CD28 antibodies

CD28 (Cluster of Differentiation 28) is a protein expressed on T cells that provide co-stimulatory signals required for T cell activation and survival. T cell stimulation through CD28 in addition to the T-cell receptor can provide a potent signal for the production of various interleukins. CD28 is the receptor for CD80 and CD86 proteins. When activated by Toll-like receptor ligands, the CD80 expression is upregulated in antigen-presenting cells (APCs). The CD86 expression on antigen-presenting cells is constitutive (expression is independent of environmental factors). An anti-CD28 antibody is an antibody that selectively binds to CD28. In various aspects, the anti-CD28antibody used herein is a neutralizing antibody.

When included in the compositions herein (i.e., a medium comprising one or more cytokine or cytokine antibody), anti-CD28 antibodies may be included in a concentration of about 0.05 to about 5 μg/mL. For example, the medium containing one or more cytokine or cytokine antibody can comprise from about 0.05 μg/mL to about 4 μg/ml, from about 0.05 μg/mL to about 3 μg/mL, from about 0.05 μg/mL to about 2 μg/mL, or from about 0.05 to about 1 μg/mL of the anti-CD28 antibody. For example, the medium comprising one or more cytokine or cytokine antibody may comprise about 0.05 μg/mL to about 3 μg/mL. In some aspects, the medium comprising one or more cytokine or cytokine antibody may comprise about 0.05 μg/mL, 0.1 μg/mL, 0.2 μg/mL, 0.3 μg/mL, 0.40 μg/mL, 0.50 μg/mL, 0.60 μg/mL, 0.7 μg/mL, 0.8 μg/mL, 0.9 μg/mL, 1.0 μg/mL, 2 μg/mL, 3 μg/mL, 4 μg/mL, or 5 μg/mL of the anti-CD28 antibody. In some aspects, the medium comprising the one or more cytokine or cytokine antibody may comprise about 0.5 μg/mL of the anti-CD28 antibody.

### IL4 and Anti-IL4 Antibodies

Interleukin-4 (IL4) is a cytokine that induces differentiation of naive helper T cells (Th0 cells) to Th2 cells. Upon activation by IL-4, Th2 cells subsequently produce additional IL-4 in a positive feedback loop. IL-4 is produced primarily by mast cells, Th2 cells, eosinophils and basophils. It is closely related and has functions similar to IL-13. Anti-IL4 antibodies used herein bind selectively to IL4.

When included in the cytokine compositions herein, IL-4 may be included at a concentration of 5 to 20 ng/mL. For example, the medium containing one or more cytokine or cytokine antibody can comprise from about 5 to 15 ng/ml, from about 5 to 12 ng/ml, from about 8 to 20 ng/mL, from about 8 to 15 ng/mL, or from about 8 to 12 ng/mL IL-4. In some aspects, the medium containing one or more cytokine or cytokine antibody can comprise about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/mL, about 18 ng/ml, about 19 ng/mL, or about 20 ng/ml IL-4. In some aspects, the medium containing one or more cytokine or cytokine antibody comprises about 10 ng/mL IL-4.

When included in the compositions herein (i.e., a medium comprising one or more cytokine or cytokine antibody), anti-IL4 antibody may be included at a concentration of about 0.5 to about 5 μg/mL. For example, the medum containing one or more cytokine or cytokine antibody can comprise from about 0.5 μg/mL to about 4 μg/ml, from about 0.5 μg/mL to about 3 μg/mL, from about 0.5 μg/mL to about 2 μg/mL, or from about 0.5 to about 1 μg/mL of the anti-IL4 antibody. In some aspects, the medium comprising one or more cytokine or cytokine antibody may comprise about 0.5 μg/mL, 1.0 μg/mL, 2 μg/mL, 3 μg/mL, 4 g/mL, or 5 μg/mL of the anti-IL4 antibody. In some aspects, the medium comprising the one or more cytokine or cytokine antibody may comprise about 1.0 μg/mL of the anti-IL4 antibody.

### IL-2

Interleukin-2 (IL-2) is a protein that regulates activity of white blood cells (e.g., leukocytes and especially lymphocytes) that are responsible for immunity. IL-2 is involved in distinguishing between foreign (“non-self”) and “self” antigens in the body and acts by binding to IL-2 receptors expressed by lymphocytes. It is released from activated CD4+ T-cells and activated CD8+ T cells.

When included in the compositions herein (i.e., a medium comprising one or more cytokine or cytokine antibody), IL-2 may be included at a concentration of 1 to 10 ng/ml. For example, in some aspects, a medium comprising one or more cytokine or cytokine antibodies can comprise from about 2 ng to 9 ng/mL, from about 3 ng to about 8 ng/mL, or from about 4 ng/ml to about 7 ng/ml of IL-2. In some aspects, the medium comprises about 1 ng/ml, about 2 ng/mL, about 3 ng/mL, about 4 ng/ml, about 5 ng/ml, about 6 ng/ml, about 7 ng/ml, about 8 ng/mL, about 9 ng/mL, or about 10 ng/ml of IL-2. In some aspects, the medium comprises about 5 ng/ml IL-2.

### IL-12

Interleukin-12 is a heterodimeric protein naturally produced by dendritic cells, macrophages, neutrophils, and human B-lymphoblastoid cells in response to antigenic stimulation.

When included in the compositions herein (i.e., a medium comprising one or more cytokine or cytokine antibody), IL-12 may be included at a concentration of 5 to 20 ng/mL. For example, the medium containing one or more cytokine or cytokine antibody can comprise from about 5 to 15 ng/mL, from about 5 to 12 ng/ml, from about 8 to 20 ng/ml, from about 8 to 15 ng/mL, or from about 8 to 12 ng/ml IL-12. In some aspects, the medium containing one or more cytokine or cytokine antibody can comprise about 5 ng/ml, about 6 ng/ml, about 7 ng/mL, about 8 ng/mL, about 9 ng/mL, about 10 ng/ml, about 11 ng/mL, about 12 ng/mL, about 13 ng/mL, about 14 ng/mL, about 15 ng/ml, about 16 ng/mL, about 17 ng/ml, about 18 ng/mL, about 19 ng/mL, or about 20 ng/mL IL-12. In some aspects, the medium containing one or more cytokine or cytokine antibody comprises about 10 ng/ml IL-12.

### IFNγ and Anti-IFNγ Antibodies

Interferon gamma (IFNγ) is a dimerized soluble cytokine that is the only member of the type II class of interferons. It regulates the immune response of a targeted cell by activating the JAK-STAT pathway and is important in both innate and adaptive immunity. It is secreted by adaptive immune cells like CD4+ Helper T (Th1) cells, natural killer cells (NK) cells and CD8+ cytotoxic T cells. Its expression levels are regulated by different cytokines. It promotes inflammation, antiviral and antibacterial activity and cell proliferation and differentiation. Anti-IFNγ antibodies bind selectively to IFNγ. In some aspects, an anti-IFNγ antibody used herein is a neutralizing antibody.

When included in the compositions herein (i.e., a medium comprising one or more cytokine or cytokine antibody), an anti-IFNγ antibody may be included at a concentration of 0.5 to about 5 μg/mL. For example, the medium containing one or more cytokine or cytokine antibody can comprise from about 0.5 μg/mL to about 4 μg/ml, from about 0.5 μg/mL to about 3 μg/mL, from about 0.5 μg/mL to about 2 μg/mL, or from about 0.5 to about 1 μg/mL of the anti-IFNγ antibody. In some aspects, the medium comprising one or more cytokine or cytokine antibody may comprise about 0.5 μg/mL, 1.0 μg/mL, 2 μg/mL, 3 μg/mL, 4 μg/mL, or 5 μg/mL of the anti-IFNγ antibody. In some aspects, the medium comprising the one or more cytokine or cytokine antibody may comprise about 1.0 μg/mL of the anti-IFNγ antibody.

### TGFβ1

TGF-β1 is a member of the transforming growth factor beta superfamily of cytokines that are collectively involved in mediating cell growth, cell proliferation, cell differentiation and aptoptosis.

When included in the compositions herein (i.e., a medium comprising one or more cytokine or cytokine antibody), TGF-β1 may be included at a concentration of 0.5 to about 5 ng/mL. For example, in some aspects, the medium comprising the one or more cytokine or cytokine antibody may comprise from about 0.5 to about 4 ng/ml, from about 0.5 to about 4 ng/mL, from about 0.5 to about 3 ng/mL, from about 1 to 5 ng/ml, from about 1 to 4 ng/ml, or from about 1 to 3 ng/mL TGF-β1. In some aspects, the medium comprising the one or more cytokine or cytokine antibody may comprise about 0.5 ng/ml, about 1.0 ng/mL, about 1.5 ng/ml, about 2.0 ng/ml, about 2.5 ng/ml, about 3.0 ng/mL, about 3.5 ng/mL, about 4.0 ng/ml, about 4.5 ng/ml, or about 5.0 ng/ml TGF-β1. In various aspects, the medium comprising the one or more cytokine or cytokine antibody may comprise about 2 ng/ml TGF-β1.

### IL-1α

Interleukin-1α (IL-1α) also known as fibroblast-activating factor (FAF), lymphocyte-activating factor (LAF), B-cell-activating factor (BAF), leukocyte endogenous mediator (LEM), epidermal cell-derived thymocyte-activating factor (ETAF), serum amyloid A inducer or hepatocyte-stimulating factor (HSP), catabolin, hemopoetin-1 (H-1), endogenous pyrogen (EP), and proteolysis-inducing factor (PIF) is a cytokine involved in inflammation. It is produced mainly by activated macrophages, neutrophils, epithelial cells and endothelial cells.

When included in the compositions herein (i.e., a medium comprising one or more cytokine or cytokine antibody), IL-1 α may be included at a concentration of 0.5 to about 5 ng/mL. For example, in some aspects, the medium comprising one or more cytokine or cytokine antibody can comprise from about 0.5 to about 4 ng/ml, from about 0.5 to about 4 ng/mL, from about 0.5 to about 3 ng/mL, from about 1 to 5 ng/mL, from about 1 to 4 ng/ml, from about 1 to 3 ng/mL, from about 2 to 5 ng/ml or from about 2 to 4 ng/ml IL-1α. In some aspects, the medium comprising the one or more cytokine or cytokine antibody may comprise about 0.5 ng/mL, about 1.0 ng/ml, about 1.5 ng/ml, about 2.0 ng/ml, about 2.5 ng/ml, about 3.0 ng/mL, about 3.5 ng/mL, about 4.0 ng/ml, about 4.5 ng/ml, or about 5.0 ng/IL-1α. In various aspects, the medium comprising the one or more cytokine or cytokine antibody may comprise about 3 ng/ml of the IL-1α.

### TNF

Tumor necrosis factor (TNF) is an adipokine and a cytokine. TNF is a member of the TNF superfamily, which consists of various transmembrane proteins with a homologous TNF domain. As an adipokine, TNF promotes insulin resistance, and is associated with obesity-induced type 2 diabetes. As a cytokine that has pleiotropic effects on various cell types. It has been identified as a major regulator of inflammatory responses and is known to be involved in the pathogenesis of some inflammatory and autoimmune diseases. TNF is also known as TNFα, cachexin and cachectin.

When included in the compositions herein (i.e., a medium comprising one or more cytokine or cytokine antibody), TNF may be included at a concentration of about 10 to about 50 ng/mL. For example, in some aspects, the medium comprising one or more cytokine or cytokine antibody can comprise from about 10 to about 45 ng/mL, from about 10 to about 40 ng/ml, from about 10 to about 35 ng/ml, from about 10 to about 30 ng/mL, from about 15 to about 50 ng/mL, from about 20 to about 50 ng/mL, from about 25 to about 50 ng/mL, from about 30 to 50 ng/ml, from about 15 to 45 ng/ml, from about 20 to 40 ng/ml, or from about 25 to 35 ng/mL TNF. For example, medium comprising one or more cytokine or cytokine antibody can comprise about 10 ng/ml, about 11 ng/mL, about 12 ng/ml, about 13 ng/ml, about 14 ng/ml, about 15 ng/mL, about 16 ng/ml, about 17 ng/ml, about 18 ng/ml, about 19 ng/ml, about 20 ng/mL, about 21 ng/mL, about 22 ng/mL, about 23 ng/ml, about 24 ng/ml, about 25 ng/mL, about 26 ng/ml, about 27 ng/ml, about 28 ng/mL, about 29 ng/ml, about 30 ng/mL, about 31 ng/mL, about 32 ng/ml, about 33 ng/mL, about 34 ng/ml, about 35 ng/ml, about 36 ng/ml, about 37 ng/ml, about 38 ng/mL, about 39 ng/ml, about 40 ng/ml, about 41 ng/mL, about 42 ng/ml, about 43 ng/mL, about 44 ng/mL, about 45 ng/ml, about 46 ng/ml, about 47 ng/ml, about 48 ng/mL, about 49 ng/mL, or about 50 ng/mL TNF. For example in some aspects, the medium comprising one or more cytokine or cytokine antibody can comprise about 30 ng/mL TNF.

### C1q

C1q is the first subcomponent of the C1 complex of the classical pathway of complement activation. Several functions have been assigned to C1q, which include antibody-dependent and independent immune functions, which are mediated by C1q receptors present on the effector cell surface

When included in the compositions herein (i.e., a medium comprising one or more cytokine or cytokine antibody), C1q may be included at a concentration of 100 to about 800 ng/mL. For example, in some aspects, the medium comprising one or more cytokine or cytokine antibody may comprise from about 100 ng/ml to about 800 ng/ml, from about 200 ng/ml to about 700 ng/mL, from about 300 ng/ml to about 600 ng/mL or from about 400 ng/ml to about 600 ng/mL C1q. In some aspects, the medium comprising one or more cytokine or cytokine antibody may comprise about 100 ng/ml, about 150 ng/ml, about 200 ng/ml, about 250 ng/mL, about 300 ng/mL, about 350 ng/ml, about 400 ng/ml, about 450 ng/ml, about 500 ng/mL, about 550 ng/ml, about 600 ng/ml, about 650 ng/ml, about 700 ng/mL, about 750 ng/mL, or about 800 ng/mL C1q. In some aspects, the medium comprising one or more cytokine or cytokine antibody may comprise about 400 ng/mL C1q.

Different cell populations useful for obtaining extracellular vesicles may be cultured in the presence of different combinations of the cytokines and antibodies thereof described above. As described above, the cell populations may comprise an astrocyte population or a T cell population.

In various aspects, the cell population comprises an astrocyte population. In some aspects, the astrocyte populations are “neurotoxic” or “reparative populations”. That is, in some aspects, the astrocyte population may be neurotoxic (“Ntx”) which herein refers to astrocytes that are activated by IL-1α, TNFα, C1q cytokines. In other aspects, the astrocyte population may be reparative (“Rep”) which herein refers to astrocytes that are activated by IL-4 and C1q cytokines.

In various aspects, the cell population can comprise astrocytes cultured in a culture medium comprising IL-4, IL-1α, TNF, C1q, or any combination thereof. For example, in some aspects, the cell population can comprise astrocytes cultured in a culture medium comprising (a) IL-1α, TNF, and/or C1q, or (b) IL-4 and/or C1q.

For example, the cell population can comprise astrocytes cultured in a culture medium comprising IL-1α, TNF, and/or C1q. In some aspects the cell population can comprise astrocytes cultured in a culture medium comprising C1q and at least one of IL-1a and TNF. In some aspects, the cell population can comprise astrocytes cultured in a culture medium comprising IL-1a and at least one of C1q and TNF. In some aspects, the cell population can comprise astrocytes cultured in a culture medium comprising TNF and at least one of C1q and IL-1α. In some aspects, the cell population can comprise astrocytes cultured in a culture medium comprising IL-1α, TNF, and C1q. In some aspects, the cell population can comprise astrocytes cultured in a culture medium comprising IL-1α, TNF, and/or C1q where the culture medium does not comprise any other cytokine or cytokine antibody, as defined herein. In certain aspects, the cell population comprising astrocytes cultured in a culture medium comprising IL-1α, TNF, and C1q comprises a neurotoxic astrocyte population.

As another example, the cell population can comprise astrocytes cultured in a culture medium comprising IL-4 and/or C1q. In some aspects, the cell population can comprise astrocytes cultured in a culture medium comprising IL-4. In some aspects the cell population can comprise astrocytes cultured in a culture medium comprising C1q. In some aspects, the cell population can comprise astrocytes cultured in a culture medium comprising IL-4 and/or C1q, where the culture medium does not comprise any other cytokine or cytokine antibody, as defined herein. In certain aspects, the cell population comprising astrocytes cultured in a culture medium comprising IL-4 and C1q comprises a reparative astrocyte population.

In various aspects, the cell population comprises a T-cells. In various aspects, the T-cells can comprise activated helper T-cells (e.g., Th1 cells or Th2 cells) or regulatory T cells. T helper 1 (Th1) cells are a subpopulation of CD4+ T helper cells that play a critical role in cell-mediated immune responses. They are characterized by their ability to secrete cytokines such as interferon-gamma (IFN-γ) and tumor necrosis factor-beta (TNF-β), which activate macrophages and enhance the cytotoxic activity of CD8+ T cells. Th1 cells are involved in the defense against intracellular pathogens, such as viruses and intracellular bacteria, and are also implicated in the pathogenesis of certain autoimmune diseases. In contrast, T helper 2 (Th2) cells are a distinct subset of CD4+ T helper cells that primarily produce cytokines such as interleukin-4 (IL-4), interleukin-5 (IL-5), and interleukin-13 (IL-13), which stimulate the production of antibodies by B cells and enhance the activity of eosinophils and mast cells. Th2 cells are involved in the defense against extracellular parasites, such as helminths, and are also implicated in the pathogenesis of allergic diseases. Regulatory T (Treg) cells are a specialized subset of CD4+ T cells that are essential for maintaining immune tolerance and preventing autoimmunity. They are characterized by the expression of the transcription factor FoxP3 and the production of the immunosuppressive cytokine transforming growth factor-beta (TGF-β). Treg cells suppress the activation and proliferation of other immune cells, including T helper cells, by various mechanisms, including the secretion of inhibitory cytokines and the induction of apoptosis in target cells.

In various aspects, the cell population comprises T cells cultured in a culture medium comprising an anti-CD28 antibody, an anti-IL4 antibody, IL-2, IL-12, an anti-IFNγ antibody, IL-4, TGFB1, or any combination thereof. For example, in some aspects, the cell population comprises T cells cultured in a culture medium comprising (a) anti-CD28 antibody, anti-IL4 antibody, IL-2, and/or IL-12; (b) anti-CD28 antibody, anti-IFNγ antibody, IL-2, and/or IL-4; or (c) anti-CD28 antibody, anti-IFNγ antibody, anti-IL4 antibody, and/or TGFβ1.

In various aspects, the cell population comprises T cells cultured in a culture medium comprising n anti-CD28 antibody, an anti-IL4 antibody, IL-2, and/or IL-12. For example, in some aspects, the cell population comprises T cells cultured in a culture medium comprising an anti-CD28 antibody. In some aspects, the cell population comprises T cells cultured in a culture medium comprising an anti-IL4 antibody. In some aspects, the cell population comprises T cells cultured in a culture medium comprising IL-2. In some aspects, the cell population comprises T cells cultured in a culture medium comprising IL-12. In some aspects, the cell population comprises T cells cultured in a culture medium comprising an anti-CD28 antibody and an anti-IL4 antibody. In some aspects, the cell population comprises T cells cultured in a culture medium comprising anti-CD28 antibody and IL-12. In some aspects, the cell population comprises T cells cultured in a culture medium comprising anti-CD28 antibody and IL-2. In some aspects, the cell population comprises T cells cultured in a culture medium comprising an anti-IL4 antibody and IL-2. In some aspects, the cell population comprises T cells cultured in a culture medium comprising an anti-IL4 antibody and IL-12. In some aspects, the cell population comprises T cells cultured in a culture medium comprising IL-2 and IL-12. In some aspects, the cell population comprises T cells cultured in a culture medium comprising anti-CD28 antibody, an anti-IL4 antibody, and IL-2. In some aspects, the cell population comprises T cells cultured in a culture medium comprising anti-CD28 antibody, an anti-IL4 antibody, and IL-12. In some aspects, the cell population comprises T cells cultured in a culture medium comprising anti-IL4 antibody, IL-2 and IL-12. In some aspects, the cell population comprises T cells cultured in a culture medium comprising an anti-CD28 antibody, IL-2 and IL-12. In various aspects, the cell population comprises T cells cultured in a culture medium comprising anti-CD28 antibody, an anti-IL4 antibody, IL-2, and IL-12. In various aspects, the cell population comprises T cells cultured in a culture medium comprising an anti-CD28 antibody, an anti-IL4 antibody, IL-2, and/or IL-12 where the culture medium does not comprise any other cytokine or cytokine antibody, as defined herein. In certain aspects, the cell population comprising T cells cultured in a culture medium comprising an anti-CD28 antibody, an anti-IL4 antibody, IL-2, and/or IL-12 comprises a Th1 helper T-cell population.

In various aspects the cell population comprises T cells cultured in a culture medium comprising an anti-CD28 antibody, an anti-IFNγ antibody, IL-2, and/or IL-4. For example, the cell population comprises T cells cultured in a culture medium comprising an anti-CD28 antibody. For example, in some aspects, the cell population comprises T cells cultured in a culture medium comprising an anti-IFNγ antibody. For example, in some aspects, the cell population comprises T cells cultured in a culture medium comprising IL-2. For example, the cell population comprises T cells cultured in a culture medium comprising IL-4. In some aspects, the cell population comprises T cells cultured in a culture medium comprising an anti-CD28 antibody and an anti-IFNγ antibody. In some aspects, the cell population comprises T cells cultured in a culture medium comprising an anti-CD28 antibody and IL-2. In some aspects, the cell population comprises T cells cultured in a culture medium comprising an anti-CD28 antibody and IL-4. In some aspects, the cell population comprises T cells cultured in a culture medium comprising an anti-IFNγ antibody and IL-2. In some aspects, the cell population comprises T cells cultured in a culture medium comprising an anti-IFNγ antibody and IL-4. In some aspects, the cell population comprises T cells cultured in a culture medium comprising IL-2 and IL-4. In some aspects, the cell population comprises T cells cultured in a culture medium comprising an anti-CD28 antibody, an anti-IFNγ antibody, and IL-2. In some aspects, the cell population comprises T cells cultured in a culture medium comprising an anti-CD28 antibody, an anti-IFNγ antibody, and IL-4. In some aspects, the cell population comprises T cells cultured in a culture medium comprising an anti-IFNγ antibody, IL-2, and IL-4. In some aspects, the cell population comprises T cells cultured in a culture medium comprising an anti-CD28 antibody, IL-2, and IL-4. In some aspects, the cell population comprises T cells cultured in a culture medium comprising an anti-CD28 antibody, an anti-IFNγ antibody, IL-2, and IL-4. In some aspects, the cell population comprises T cells cultured in a culture medium comprising an anti-CD28 antibody, an anti-IFNγ antibody, IL-2, and/or IL-4, where the culture medium does not comprise any other cytokine or cytokine antibody, as defined herein. In certain aspects, a cell population comprising T cells cultured in a culture medium comprising an anti-CD28 antibody, an anti-IFNγ antibody, IL-2, and/or IL-4 comprises a Th2 helper T-cell population.

In various aspects the cell population comprises T cells cultured in a culture medium comprising an anti-CD28 antibody, an anti-IFNγ antibody, an anti-IL4 antibody, and/or TGFB1. For example, in some aspects, the T-cell population may be derived by culturing T-cells in a culture medium comprising an anti-CD28 antibody. In some aspects, the T-cell population may be derived by culturing T-cells in a culture medium comprising an anti-IFNγ antibody. In some aspects, the T-cell population may be derived by culturing T-cells in a culture medium comprising an anti-IL4 antibody. In some aspects, the T-cell population may be derived by culturing T-cells in a culture medium comprising TGFB1. In some aspects, the T-cell population may be derived by culturing T-cells in a culture medium comprising an anti-CD28 antibody and an anti-IFNγ antibody. In some aspects, the T-cell population may be derived by culturing T-cells in a culture medium comprising an anti-CD28 antibody and an anti-IL4 antibody. In some aspects, the T-cell population may be derived by culturing T-cells in a culture medium comprising an anti-CD28 antibody and TGFB1. In some aspects, the T-cell population may be derived by culturing T-cells in a culture medium comprising an anti-IFNγ antibody and an anti-IL4 antibody. In some aspects, the T-cell population may be derived by culturing T-cells in a culture medium comprising an anti-IFNγ antibody and TGFβ1. In some aspects, the T-cell population may be derived by culturing T-cells in a culture medium comprising an anti-IL4 antibody and TGFβ1. In some aspects, the T-cell population may be derived by culturing T-cells in a culture medium comprising an anti-CD28 antibody, an anti-IL4 antibody and TGFB1. In some aspects, the T-cell population may be derived by culturing T-cells in a culture medium comprising an anti-IFNγ antibody, an anti-IL4 antibody and TGFB1. In some aspects, the T-cell population may be derived by culturing T-cells in a culture medium comprising an anti-CD28 antibody, an anti-IFNγ antibody and TGFβ1. In some aspects, the T-cell population may be derived by culturing T-cells in a culture medium comprising an anti-CD28 antibody, an anti-IFNγ antibody and an anti-IL4 antibody. In various aspects the cell population comprises T cells cultured in a culture medium comprising an anti-CD28 antibody, an anti-IFNγ antibody, an anti-IL4 antibody, and TGFB1. In various aspects the cell population comprises T cells cultured in a culture medium comprising an anti-CD28 antibody, an anti-IFNγ antibody, an anti-IL4 antibody, and/or TGFβ1, where the culture medium does not comprise any other cytokine or cytokine antibody, as defined herein. In certain aspects, a cell population comprising T cells cultured in a culture medium comprising an anti-CD28 antibody, an anti-IFNγ antibody, an anti-IL4 antibody, and/or TGFβ1 comprises a regulatory T-cell population.

In any of the aforementioned aspects, when the cell populations are cultured in a medium comprising an anti-CD28 antibody, an anti-IL4 antibody, IL-2, IL-12, an anti-IFNγ antibody, IL-4, TGFβ1, IL-1α, TNF, C1q, or any combination thereof, the medium can comprise from about 0.05 to about 3 μg/mL of the anti-CD28 antibody, about 0.5 to about 5 μg/mL of the anti-IL4 antibody, about 1 to about 10 ng/ml of the IL-2, about 5 to about 20 ng/ml of the IL-12, about 0.5 to about 5 μg/mL of the anti-IFNγ antibody, about 5 to about 20 ng/mL of the IL-4, about 0.5 to about 5 ng/ml of the TGFβ1, about 1 to about 5 ng/ml of the IL-1α, about 10 to about 50 ng/ml of the TNF, about 100 to about 800 ng/mL of the C1q, or any combination thereof. In some aspects, when the cell populations are cultured in a medium comprising an anti-CD28 antibody, an anti-IL4 antibody, IL-2, IL-12, an anti-IFNγ antibody, IL-4, TGFβ1, IL-1α, TNF, C1q, or any combination thereof as described above, the medium comprises about 0.5 μg/mL of the anti-CD28 antibody, about 1 μg/mL of the anti-IL4 antibody, about 5 ng/ml of the IL-2, about 10 ng/ml of the IL-12, about 1 μg/mL of the anti-IFNγ antibody, about 10 ng/ml of the IL-4, about 2 ng/mL of the TGFβ1, about 3 ng/ml of the IL-1α, about 30 ng/ml of the TNF, about 400 ng/ml of the C1q, or any combination thereof.

In various aspects, the cells may be cultured in the medium comprising the one or more cytokines or cytokine antibodies for a period of time. For example, the cells may be cultured in the medium comprising the one or more cytokines or cytokine antibodies for at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some aspects, the cells may be cultured in the medium comprising the one or more cytokines or cytokine antibodies for at least 24 hours. In some aspects, the cells are cultured in the medium comprising the one or more cytokines or cytokine antibodies for about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some aspects, the cells may be cultured in the medium comprising the one or more cytokines or cytokine antibodies for about 24 hours. In some aspects, the cells are cultured in the medium comprising the one or more cytokines or cytokine antibodies for 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some aspects, the cells may be cultured in the medium comprising the one or more cytokines or cytokine antibodies for 24 hours.

In various aspects, the medium comprising the one or more cytokines or cytokine antibodies described herein comprises an extracellular vesicle (EV) collecting media as described below.

### Isolating Extracellular Vesicles

Extracellular vesicles may be isolated from the cell populations above using conventional methods. In various aspects, isolating extracellular vesicles may comprise culturing a cell population in an extracellular vesicle (EV) collecting media. In some aspects, the EV collecting media can comprise a Dulbecco's Modified Eagle Medium (DMEM) modified with additional components to promote generation and recovery of EVs from a cell population. For example, the EV collecting media may further comprise FBS (fetal bovine serum), hydrocortisone, an antibacterial agent (e.g., Pen/Strep), glutamine, insulin, NaPyruvate, N-acetyl-L-cysteine or any combination thereof. Accordingly, in some aspects, the EV collecting media can comprise Dulbecco's Modified Eagle Medium (DMEM), FBS (fetal bovine serum), hydrocortisone, an antibacterial agent (e.g., Pen/Strep), glutamine, insulin, NaPyruvate, N-acetyl-L-cysteine, or a combination of any thereof. In some aspects, the EV collecting media can comprise Dulbecco's Modified Eagle Medium (DMEM), FBS (fetal bovine serum), hydrocortisone, an antibacterial agent (e.g., Pen/Strep), glutamine, insulin, NaPyruvate, and N-acetyl-L-cysteine.

In various aspects, the EV collecting media comprises the media comprising the one or more cytokines or cytokine antibodies as described above. That is, in some aspects, a cell population may be cultured in an EV collecting media (e.g., comprising Dulbecco's Modified Eagle Medium (DMEM), FBS (fetal bovine serum), hydrocortisone, an antibacterial agent (e.g., Pen/Strep), glutamine, insulin, NaPyruvate, and N-acetyl-L-cysteine) and further comprising one or more cytokines or cytokine antibodies described above.

Accordingly, in view of the foregoing aspects, extracellular vesicles derived or collected from a cell population are provided. These extracellular vesicles (EVs) are then immobilized on various microparticles to form EV loaded microparticles as described further below.

### (b) Hydrogel Microparticles

As noted, aspects of the present disclosure are directed to compositions containing extracellular vesicles (EVs) immobilized on hydrogel microparticles.

In various aspects, the hydrogel microparticles can comprise a polymer. Suitable polymers, non-limiting examples of which include hyaluronic acid (HA), chitosan, heparin, alginate, gelatin, fibrin, collagen, MATRIGEL®, polyvinyl alcohol, polyethylene glycol, sodium polyacrylate, acrylate polymers, copolymers, dithiol polymers (e.g., acrylamide), click-based composite hydrogels poly (ethylene glycol)-diacrylate, poly(ethylene glycol)-vinyl sulfone, or any combination thereof. In some aspects, the hydrogel microparticles can comprise hyaluronic acid (HA), poly (ethylene glycol), gelatin, alginate, collagen, Methyl cellulose, or any combination thereof. In some aspects, the hydrogel microparticles can comprise hyaluronic acid (HA). In some aspects, polymers can be functionalized polymers. Functionalization of the polymer can be confirmed with ¹H nuclear magnetic resonance spectroscopy, mass spectroscopy, Elman's reagent, UV-Vis spectroscopy, infrared spectroscopy, and other methods known to those skilled in the art.

In some aspects, the hydrogel polymer in the microparticle can be modified with one or more functional groups. In some aspects, hydrogel polymer can be modified with a functional group norbornene, acrylamide, tetrazine, sulfate, cyclodextrin, adamantane, vinyl sulfone, acrylate, allyl, azide, alkyne, thiol, PEG, or any combination thereof. In some aspects, hydrogel microparticle can comprise HA modified with acrylamide functional groups (HA-AC). In some aspects, hydrogel microparticle can comprise HA modified with norbornene functional group (HA-NB).

In further aspects, the hydrogel polymer can be further modified with a cell adhesion peptide. In some aspects the cell adhesion peptide can be RGD ligand (RGDSP, SEQ ID NO: 1 or RGDSPGERCG, SEQ ID NO:2). In other aspects, the hydrogel polymer can be modified with other ligands, Q peptide, K peptide, or any combination thereof. In some aspects, hydrogel scaffold can comprise HA modified with one or more RGD ligands (RGDSP (SEQ ID NO: 1) or RGDSPGERCG (SEQ ID NO: 2)), one or more Q-peptides (NQEQVSPLGGERCG, SEQ ID NO: 3), one or more K-peptides (FKGGERCG, SEQ ID NO: 4), or any combination thereof.

In accord with the foregoing, functionalized microparticles may be generate comprising hyaluronic acid functionalized with norbonene functional groups. These prepared microparticles may be further modified by converting the functional group (e.g., the norbonene functional group) to a strained alkyne. For example, dibenzocyclooctyne-tratazine (DBCO-tetrazine) may be used to convert free norbonene groups to DBCO groups using a Diels-Alder click reaction.

### (c) Extracellular Vesicle-Hydrogel Microparticles (EV-HMP)

As noted, aspects of the present disclosure are directed to compositions containing extracellular vesicles (EVs) immobilized on hydrogel microparticles (e.g., EV-HMPs). These compositions are provided herein as an extracellular vesicle hydrogel microparticle (EV-HMP), the microparticle comprising: (i) a hydrogel microparticle (HMP) and (ii) an extracellular vesicle (EV) derived or collected from a cell population, where the extracellular vesicle is immobilized to the HMP. The cell populations can be any of those disclosed herein. For example, the cell populations may comprise astrocytes and/or T-cells.

In various aspects, the EVs are immobilized to the HMPs via a Diels-Alder click reaction between an strained alkyne functional group (e.g., “strained alkyne”) on the HMP and an azide containing moiety on the EV. As used herein, the term “strained alkyne” refers to a type of alkyne molecule where the triple bond between the carbon atoms is under significant strain due to the presence of bulky or electron-withdrawing groups that cause the carbon-carbon triple bond to deviate from the linear configuration. The strain in strained alkynes arises from the steric repulsion between the substituents attached to the carbon atoms of the triple bond. The presence of these substituents leads to bond angles that are less than the ideal 180 degrees, which results in increased energy and instability of the molecule. Therefore, strained alkynes have high reactivity, which makes them suitable to be reacted with an azide group under physiological conditions without the need of toxic catalyst. Suitable strained alkynes may include, for example, dibenzocyclooctyne (DBCO) bicyclo[6.1.0]non-4-yn-9-ylmethanol (BCN), azadibenzocyclooctyne (ADIBO), cyclooctyne (OCT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or any combination thereof. In various aspects, the strained alkyne can comprise dibenzocyclooctyne (DBCO). In certain aspects, a norborene-HA microparticle may be prepared according to the methods above and then reacted with DBCO-triazine to form a DBCO-functionalized microparticle. Azide reactive groups on the EV may be provided by, for example, labeling an EV with an azido sugar. This can be accomplished, for example, by culturing a cell population with the azido sugar before isolating the extracellular vesicle. As the cell population metabolizes the azido sugar it presents it on its plasma membrane, which is then incorporated into extracellular vesicles produced by the cell population. Exemplary azido sugars that may be used include, for example, azidoacetylmannosamine-tetraacylated (Ac4MannAz), 9-azido sialic acid, 6-Azide-Trehalose (6-TreAz), 8-Azido-3,8-dideoxy-D-manno-octulosonic acid (Kdo Azide), 9-azido-9-deoxy-N-acetylneuraminic acid (9AzNeu5Ac), N-azidoacetylglucosamine-tetraacylated (Ac4GlcNAz), N-azidoacetylgalactosamine-tetraacylated (Ac4GalNAz), or 6-azido-6-deoxy-N-acetyl-glucosamine triacylated (Ac3-6AzGlcNAc). For example, in some aspects, the azido sugar may be azidoacetylmannosamine-tetraacylated (Ac4MannAz).

Accordingly, in various aspects, the EVs may be labeled with an azido sugar (e.g., azidoacetylmannosamine-tetraacylated (Ac4MannAz)) and the hydrogel microparticles may be functionalized with a strained alkyne (i.e., DBCO) such that the azido sugar and the strained alkyne react to form a stable triazole linkage. In various aspects, the reaction between the azido sugar and the strained alkyne is a biorthogonal reaction which herein refers to a a chemical reaction that can occur inside living organisms without interfering with the natural biological processes or causing any harm to the cells. One of the key features of a bioorthogonal reaction is its selectivity. It should be specific to the biomolecule of interest and not react with other molecules present in the cell. Furthermore, it should be fast, efficient, and occur under mild conditions, such as neutral pH, low temperature, or in the presence of water.

Accordingly, in some aspects, an extracellular vesicle hydrogel microparticle (HMP) is provided comprising an extracellular vesicle immobilized to a hydrogel microparticle via a stable triazole linkage. In various aspects, the stable triazole linkage is formed from a biorthogonal reaction between the azido sugar and strained alkyne.

In accord with various aspects described above, an extracellular vesicle hydrogel microparticle (EV-HMP) is provided wherein the EV-HMP comprises: (i) a hydrogel microparticle (HMP) functionalized with a strained alkyne and (ii) an extracellular vesicle (EV) labeled with an azido sugar derived or collected from a cell population, wherein the cell population comprises astrocytes or T-cells cultured in a medium comprising at least one of an anti-CD28 antibody, an anti-IL4 antibody, IL-2, IL-12, an anti-IFNγ antibody, IL-4, TGFβ1, IL-1α, TNF, C1q, or any combination thereof, and wherein the extracellular vesicle is immobilized to the HMP via a stable triazole linkage between the strained alkyne and the azido sugar.

Methods for preparing an EV-HMP are provided in more detail below.

Also provided is an EV-HMP suspension comprising two or more EV-HMPs herein. The term EV-HMP suspension (also known as a plurality of EV-HMPs) refers to a collection of two or more EV-HMPs, optionally in the presence of an inert carrier (i.e., water, buffer, saline or the like).

### (d) Extracellular Vesicle Microporous Annealed Particle (EV-MAP) Hydrogel

Further aspects of the present disclosure are directed to extracellular microporous annealed particle (EV-MAP) hydrogels, where the EV-MAPs comprise one or more EV-HMPs (e.g., two or more HMPs) and a crosslinker.

Suitable crosslinkers include, for example, any crosslinker comprising two or more azide groups. In some aspects, the crosslinker comprises tetra-arm PEG-azide.

In various aspects, a portion of the EV-HMPs in the composition are linked by the crosslinker. In various aspects, at least two EV-HMPs are linked by the crosslinker. For example, in some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the EV-HMPs in the composition are linked by the crosslinker. In some aspects about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% of the EV-HMPs in the composition are linked by the crosslinker. In some aspects 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the EV-HMPs in the composition are linked by the crosslinker.

### II. Pharmaceutical Formulations

The present disclosure is also directed to pharmaceutical compositions (formulations) comprising one or more EV-HMPs and/or EV-MAPs described above.

In some aspects, a pharmaceutical composition is provided comprising one or more EV-HMPs described herein and a pharmaceutically appropriate carrier. In some aspects, a pharmaceutical composition is provided comprising two or more EV-HMPs described herein and a pharmaceutically appropriate carrier. In further aspects, a pharmaceutical composition is provided comprising an EV-MAP hydrogel as described herein and a pharmaceutically appropriate carrier.

By “pharmaceutically acceptable” it is meant a material that is not biologically or otherwise undesirable, i.e., the material can be administered to a subject along with the isolated nucleic acid or vector without causing any undesirable biological effects such as toxicity. Thus, such a pharmaceutical composition can be used, for example, in transfection of a cell ex vivo or in administering an isolated nucleic acid or vector directly to a subject.

In some embodiments, compositions of the present disclosure comprise, consist of, or consist essentially of one or more EV-HMPs or EV-MAP hydrogel, as provided herein, and/or a pharmaceutically acceptable carrier and/or excipient, and, optionally, other medicinal agents, pharmaceutical agents, stabilizing agents, buffers, carriers, adjuvants, diluents, etc. For injection, the carrier will typically be a liquid. For various methods of administration, the carrier can be either solid or liquid. For injectable methods of administration (i.e., via stereotaxic injection), the carrier may be a liquid.

The compositions can also comprise other ingredients such as diluents and adjuvants. Acceptable carriers, diluents and adjuvants are nontoxic to recipients and are preferably inert at the dosages and concentrations employed, and can include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

The pharmaceutical carriers, diluents or excipients suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some embodiments, sterile injectable solutions are prepared by incorporating one more EV-HMPs in the required amount in the appropriate solvent with various other ingredients enumerated above, as required, followed by filter sterilization. In some embodiments, sterile injectable solutions are prepared by an EV-MAP hydrogel in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. A dispersion of EV-HMPs can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

Pharmaceutical compositions can be prepared as injectable formulations or as topical formulations to be delivered to the subject by transdermal transport. Numerous formulations for both intramuscular injection and transdermal transport have been previously developed and can be used in the practice of the disclosure. The EV-HMPs and/or EV-MAPs of the present disclosure can be used with any pharmaceutically acceptable carrier and/or excipient for ease of administration and handling.

### III. Methods

Further aspects of the present disclosure are related to methods of culturing cell populations in the presence of one or more cytokines or cytokine antibodies, methods of making EV-HMPs and EV-MAPs using EVs derived from these cell populations, and then using the EV-HMPs and EV-MAPs in various therapeutic applications.

### (a) Methods of Making Cell Populations

Various methods of the present disclosure comprise or are directed to methods of making a cell population. In various aspects, these methods comprise culturing a cell population in a culture medium comprising at least one cytokine or cytokine antibody. These methods are described or referenced in Section (II)(a) above. In various aspects, a method of making a reactive astrocyte population is provided, the method comprising culturing an astrocyte population in a culture medium comprising at least one cytokine or cytokine antibody. In other aspects, a method of making an activated T-cell population is provided, the method comprising culturing a T-cell population in a culture medium comprising at least one cytokine or cytokine antibody.

Suitable combinations of cytokines and cytokine antibodies that may be included in these culture medium to derive the cell populations are described in more detail in Section (II)(a) and also referenced in Section (III)(b) below.

Briefly, in some aspects, the method comprises preparing a reactive astrocyte population by culturing astrocytes in a culture medium comprising IL-4, IL-1α, TNF, C1q, or any combination thereof. For example, the method can comprise culturing astrocytes in a culture medium comprising (a) IL-1α, TNF, and/or C1q; or (b) IL-4 and/or C1q. In some aspects, the method comprises preparing a neurotoxic astrocyte population by culturing astrocytes in a culture medium comprising (a) IL-1α, TNF, and/or C1q. In some aspects, the method comprises preparing a reparative astrocyte population by culturing astrocytes in a culture medium comprising (b) IL-4 and/or C1q.

In other aspects, the method comprises preparing an activated T-cell population by culturing T-cells in a culture medium comprising anti-CD28 antibody, anti-IL4 antibody, IL-2, IL-12, anti-IFNγ antibody, IL-4, TGFβ1, or any combination thereof. For example, the method can comprise culturing T-cells in a culture medium comprising an (a) anti-CD28 antibody, anti-IL4 antibody, IL-2, and/or IL-12; (b) anti-CD28 antibody, anti-IFNγ antibody, IL-2, and/or IL-4; or (c) anti-CD28 antibody, anti-IFNγ antibody, anti-IL4 antibody, and/or TGFβ1. In some aspects, the method comprises preparing a Th1 helper T cell population by culturing T-cells in a culture medium comprising an (a) anti-CD28 antibody, anti-IL4 antibody, IL-2, and/or IL-12. In some aspects, the method comprises preparing a Th2 helper T cell population by culturing T-cells in a culture medium comprising (b) anti-CD28 antibody, anti-IFNγ antibody, IL-2, and/or IL-4. In some aspects, the method comprises preparing a Regulatory T cell population by culturing T-cells in a culture medium comprising (c) anti-CD28 antibody, anti-IFNγ antibody, anti-IL4 antibody, and/or TGFβ1.

In any of these methods, the culture medium described can comprise from about 0.05 to about 3 μg/mL of the anti-CD28 antibody, about 0.5 to about 5 μg/mL of the anti-IL4 antibody, about 1 to about 10 ng/ml of the IL-2, about 5 to about 20 ng/ml of the IL-12, about 0.5 to about 5 μg/mL of the anti-IFNγ antibody, about 5 to about 20 ng/ml of the IL-4, about 0.5 to about 5 ng/ml of the TGFβ1, about 1 to about 5 ng/ml of the IL-1α, about 10 to about 50 ng/ml of the TNF, about 100 to about 800 ng/ml of the C1q, or any combination thereof. In some aspects, the culture medium described above can comprise at least one cytokine or cytokine antibody comprises about 0.5 μg/mL of the anti-CD28 antibody, about 1 μg/mL of the anti-IL4 antibody, about 5 ng/mL of the IL-2, about 10 ng/ml of the IL-12, about 1 μg/mL of the anti-IFNγ antibody, about 10 ng/ml of the IL-4, about 2 ng/mL of the TGFβ1, about 3 ng/mL of the IL-1α, about 30 ng/mL of the TNF, about 400 ng/ml of the C1q, or any combination thereof.

The methods can comprise culturing the cell populations in the appropriate media for at least for at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some aspects, the methods can comprise culturing the cells in the medium comprising the one or more cytokines or cytokine antibodies for at least 24 hours. In some aspects, the cells are cultured in the medium comprising the one or more cytokines or cytokine antibodies for about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some aspects, the method can comprise culturing the cells in the medium comprising the one or more cytokines or cytokine antibodies for about 24 hours. In some aspects, the cells are cultured in the medium comprising the one or more cytokines or cytokine antibodies for 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.

Reactive cell populations (e.g., neurotoxic astrocytes, reparative astrocytes, Th1 helper T cells, Th2 helper T cells, or regulatory T cells) prepared according to these methods can be evaluated for specific markers or activity appropriate for the reactive cell population according to standard methods in the art.

### (b) Methods of Making EV-HMPs

In general, a method of making an EV-HMP provided herein can comprise: (a) isolating an extracellular vesicle labeled with an azido sugar (azido-EV) from a cell population; and (b) contacting the azido-EV with a hydrogel microparticle (HMP) functionalized with a strained alkyne so that the azido sugar of the azido-EV forms a stable triazole linkage with the strained alkyne of the HMP to make the extracellular vesicle hydrogel microparticle (EV-HMP). Each step is described in more detail below.

Step (a)—Isolating an Extracellular Vesicle Labeled with an Azido Sugar (Azido-EV) from a Cell Population.

In various aspects, an extracellular vesicle may be labeled with an azido sugar by culturing the cell population in an azido sugar before isolating the extracellular vesicle. In some aspects, the cell population may be cultured in the presence of an azido sugar for at least 5 hours, at least 10 hours, at least 15 hours, at least 20 hours, at least 25 hours, and at least 30 hours. In some aspects, the cell population may be cultured in the presence of the azido sugar for at least 24 hours. In various aspects, the cell population is cultured in the presence of an azido sugar at an elevated temperature (e.g., at about 37° C.).

Suitable azido sugars can include N-azidoacetylmannosamine-tetraacylated (Ac4MannAz), 9-azido sialic acid, 6-Azide-Trehalose (6-TreAz), 8-Azido-3,8-dideoxy-D-manno-octulosonic acid (Kdo Azide), 9-azido-9-deoxy-N-acetylneuraminic acid (9AzNeu5Ac), N-azidoacetylglucosamine-tetraacylated (Ac4GlcNAz), N-azidoacetylgalactosamine-tetraacylated (Ac4GalNAz), 6-azido-6-deoxy-N-acetyl-glucosamine triacylated (Ac3-6AzGlcNAc), or any combination thereof.

In various aspects, the step (a) comprises isolating more than one azido-EV from the cell population and therefore comprises forming an azido-EV suspension which comprises more than one EV metabolically labeled with the azido sugar according to the methods above.

In various aspects, the cell population of step (a) may be cultured in a medium comprising at least one cytokine or cytokine antibody. The medium comprising at least one cytokine or cytokine antibody may comprise any of the media described above to culture a cell population (e.g., see Section (II) (a) and Section (III) (a) above).

The medium comprising at least one cytokine or cytokine antibody used to collect EVs for use in the disclosed compositions can also comprise the one or more cytokine or cytokine antibody disclosed above to culture the cell populations above (e.g., astrocytes or T-cells). Accordingly, in some non-limiting aspects, the medium comprising at least one cytokine or cytokine antibody may comprise an anti-IL4 antibody, IL-2, IL-12, an anti-IFNγ antibody, IL-4, TGFβ1, IL-1α, TNF, C1q, or any combination thereof.

In some aspects, the medium comprising at least one cytokine or cytokine antibody can comprise IL-4, IL-1α, TNF, C1q, or any combination thereof. In some aspects, the medium comprising at least one cytokine or cytokine antibody can comprise IL-1α, TNF, and C1q. In some aspects, the medium comprising at least one cytokine or cytokine antibody can comprise IL-4, C1q, or any combination thereof. In some aspects, the medium comprising at least one cytokine or cytokine antibody can comprise IL-4, and C1q.

In some aspects, the medium comprising at least one cytokine or cytokine antibody can comprise anti-CD28 antibody, anti-IL4 antibody, IL-2, IL-12, anti-IFNγ antibody, IL-4, TGFβ1, or any combination thereof. In some aspects, the medium comprising at least one cytokine or cytokine antibody can comprise anti-CD28 antibody, anti-IL4 antibody, IL-2, IL-12, or any combination thereof. In some aspects, the medium comprising at least one cytokine or cytokine antibody can comprise anti-CD28 antibody, anti-IL4 antibody, IL-2, and IL-12. In some aspects, the medium comprising at least one cytokine or cytokine antibody can comprise anti-CD28 antibody, anti-IFNγ antibody, IL-2, IL-4, or any combination thereof. In some aspects, the medium comprising at least one cytokine or cytokine antibody can comprise anti-CD28 antibody, anti-IFNγ antibody, IL-2, and IL-4. In some aspects, the medium comprising at least one cytokine or cytokine antibody can comprise anti-CD28 antibody, anti-IFNγ antibody, anti-IL4 antibody, TGFβ1, or any combination thereof. In some aspects, the medium comprising at least one cytokine or cytokine antibody can comprise anti-CD28 antibody, anti-IFNγ antibody, anti-IL4 antibody and TGFβ1.

In various aspects, isolating an extracellular vesicle from the cell population comprises culturing the cell population in an extracellular vesicle (EV) collection media (sometimes referred to as an EV collecting media). Suitable EV collection media are known in the art. In some aspects, the EV collection media provides at least one component from one or more an energy source, usually in the form of a carbohydrate such as glucose, all essential amino acids, and usually the basic set of twenty amino acids plus cysteine, vitamins and/or other organic compounds required at low concentrations, free fatty acids, trace elements, where trace elements are defined as inorganic compounds or naturally occurring elements that are typically required at very low concentrations, usually in the micromolar range, or any combination thereof. The EV collection media may optionally be supplemented with one or more components, non-limiting examples of which include hormones and other growth factors (e.g., insulin, transferrin, and epidermal growth factor), salts and buffers (e.g., calcium, magnesium, and phosphate), nucleosides and bases (e.g., adenosine, thymidine, and hypoxanthine), and protein and tissue hydrolysates. In some aspects, EV collection media can comprise Ham's F10, Minimal Essential Medium (MEM), RPMI-1640, Dulbecco's, Modified Eagle's Medium (DMEM), Ex-cell GTM3, Freestyle 5FM293, Hyclone 5FM293, RPMI1640 medium, GIT medium, Ex-cell 302 medium, IMDM medium, Hybridoma-SFM or any combination thereof. In some aspects, the EV collection media can comprise DMEM. In some aspects, the EV collection media can further comprise fetal bovine serum (FBS), hydrocortisone, an antibacterial agent (e.g., Pen/Strep), glutamine, insulin, NaPyruvate, N-acetyl-L-cysteine or any combination thereof.

In some aspects, the EV collecting media used to collect EVs for use in the disclosed compositions can also comprise the one or more cytokine or cytokine antibody disclosed above. Accordingly, in some non-limiting aspects, the EV-collecting media may comprise an anti-IL4 antibody, IL-2, IL-12, an anti-IFNγ antibody, IL-4, TGFβ1, IL-1α, TNF, C1q, or any combination thereof.

In some aspects, the EV collecting media can comprise IL-4, IL-1α, TNF, C1q, or any combination thereof. In some aspects, the EV collecting media can comprise IL-1α, TNF, and C1q. In some aspects, the EV collecting media can comprise IL-4, C1q, or any combination thereof. In some aspects, the EV collecting media can comprise IL-4 and C1q.

In some aspects, the EV collecting media can comprise anti-CD28 antibody, anti-IL4 antibody, IL-2, IL-12, anti-IFNγ antibody, IL-4, TGFβ1, or any combination thereof. In some aspects, the EV collecting media can comprise anti-CD28 antibody, anti-IL4 antibody, IL-2, IL-12, or any combination thereof. In some aspects, the EV collecting media can comprise anti-CD28 antibody, anti-IL4 antibody, IL-2, and IL-12. In some aspects, the EV collecting media can comprise anti-CD28 antibody, anti-IFNγ antibody, IL-2, IL-4, or any combination thereof. In some aspects, the EV collecting media can comprise anti-CD28 antibody, anti-IFNγ antibody, IL-2, and IL-4. In some aspects, the EV collecting media can comprise anti-CD28 antibody, anti-IFNγ antibody, anti-IL4 antibody, TGFβ1, or any combination thereof. In some aspects, the EV collecting media can comprise anti-CD28 antibody, anti-IFNγ antibody, anti-IL4 antibody and TGFβ1.

In any of the foregoing aspects, the EV collection media may further comprise DMEM, fetal bovine serum (FBS), hydrocortisone, an antibacterial agent (e.g., Pen/Strep), glutamine, insulin, NaPyruvate, N-acetyl-L-cysteine or any combination thereof.

Accordingly, in some aspects, the EV collecting media can comprise DMEM, fetal bovine serum (FBS), hydrocortisone, an antibacterial agent (e.g., Pen/Strep), glutamine, insulin, NaPyruvate, N-acetyl-L-cysteine, an anti-IL4 antibody, IL-2, IL-12, an anti-IFNγ antibody, IL-4, TGFβ1, IL-1α, TNF, C1q, or any combination thereof.

In some aspects, the EV collecting media can comprise DMEM, fetal bovine serum (FBS), hydrocortisone, an antibacterial agent (e.g., Pen/Strep), glutamine, insulin, NaPyruvate, N-acetyl-L-cysteine, IL-4, IL-1α, TNF, C1q, or any combination thereof. In some aspects, the EV collecting media can comprise DMEM, fetal bovine serum (FBS), hydrocortisone, an antibacterial agent (e.g., Pen/Strep), glutamine, insulin, NaPyruvate, N-acetyl-L-cysteine, IL-1a, TNF, and C1q. In some aspects, the EV collecting media can comprise DMEM, fetal bovine serum (FBS), hydrocortisone, an antibacterial agent (e.g., Pen/Strep), glutamine, insulin, NaPyruvate, N-acetyl-L-cysteine, IL-4, C1q, or any combination thereof. In some aspects, the EV collecting media can comprise DMEM, fetal bovine serum (FBS), hydrocortisone, an antibacterial agent (e.g., Pen/Strep), glutamine, insulin, NaPyruvate, N-acetyl-L-cysteine, IL-4, and C1q.

In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IL4 antibody, IL-2, IL-12, anti-IFNγ antibody, IL-4, TGFβ1, or any combination thereof. In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IL4 antibody, IL-2, IL-12, or any combination thereof. In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IL4 antibody, IL-2, and IL-12. In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IFNγ antibody, IL-2, IL-4, or any combination thereof. In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IFNγ antibody, IL-2, and IL-4. In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IFNγ antibody, anti-IL4 antibody, TGFβ1, or any combination thereof. In some aspects, the EV collecting media can comprise DMEM, fetal bovine serum (FBS), hydrocortisone, an antibacterial agent (e.g., Pen/Strep), glutamine, insulin, NaPyruvate, anti-CD28 antibody, anti-IFNγ antibody, anti-IL4 antibody, and TGFβ1.

In various aspects, the cell population is cultured in the medium comprising the one or more cytokines or cytokine antibodies for a period of time. For example, the cell population may be cultured in the medium comprising the one or more cytokines or cytokine antibodies for at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some aspects, the method can comprise culturing the cells in the medium comprising the one or more cytokines or cytokine antibodies for at least 24 hours. In some aspects, the cells are cultured in the medium comprising the one or more cytokines or cytokine antibodies for about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some aspects, the method can comprise culturing the cells in the medium comprising the one or more cytokines or cytokine antibodies for about 24 hours. In some aspects, the cells are cultured in the medium comprising the one or more cytokines or cytokine antibodies for 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some aspects, the method can comprise culturing the cells in the medium comprising the one or more cytokines or cytokine antibodies for 24 hours. In any of these aspects, the medium comprising the one or more cytokines or cytokine antibodies can be an EV collecting media as described above.

Step (b) Contacting the Azido-EV with a Hydrogel Microparticle (HMP) Functionalized with a Strained Alkyne so that the Azido Sugar of the Azido-EV Forms a Stable Triazole Linkage with the Strained Alkyne of the HMP

In various aspects, methods provided herein comprise contacting the azido-EV with a hydrogel microparticle (HMP) functionalized with at least one strained alkyne on a surface so that the azido sugar of the azido-EV forms a stable triazole linkage with the strained alkyne of the HMP.

Suitable hydrogel microparticles (HMPs) that can be used in this method are described above. In some aspects, the HMP comprises hyaluronic acid (HA). In some aspects, the HMP used in these methods may be prepared from an HMP comprising a suitable polymer functionalized with a functional group that may be converted to an strained alkyn. For example, in some aspects, the strained alkyne may comprise DBCO bicyclo[6.1.0]non-4-yn-9-ylmethanol (BCN), azadibenzocyclooctyne (ADIBO), cyclooctyne (OCT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or any combination thereof. In some cases, the HMP may comprise an HMP formed from hyaluronic acid functionalized with norbonene which can be reacted with DBCO-triazine to form the HMP functionalized with DBCO.

In some aspects, hydrogel microparticles can be prepared by crosslinking hydrogel polymers with a polymer crosslinker. Polymer crosslinkers for use in the hydrogel microparticle preparation are known by those skilled in the art. In some aspects, crosslinker can be a multifunctional crosslinker for example, a bifunctional polymer crosslinker. Non-limiting examples of bi-functional polymer crosslinkers and multifunctional polymer crosslinkers include polyethylene glycol dithiol (PEG-DT), protease-degradable crosslinkers and multi-arm poly(ethylene glycol) terminated with thiol (e.g., 4-arm PEG terminated with thiol). In some aspects, the crosslinker can comprise a protease-degradable crosslinker for example, matrix metalloproteinase (MMP)-degradable crosslinkers (MMP-1-degradable crosslinkers, MMP-2-degradable crosslinkers, MMP-9-degradable crosslinkers), Omi degradable sequences, or Heat-Shock Protein degradable sequences. In another aspect, the crosslinker sequences are hydrolytically degradable natural and synthetic polymers e.g., heparin, alginate, poly(ethylene glycol), polyacrylamides, polymethacrylates, polyesters, polyamides, and polyurethanes. In some aspects, the crosslinker can comprise a MMP-degradable crosslinker. In some aspects, hydrogel microparticles can be prepared by crosslinking hydrogel polymer with a MMP dithiol crosslinker (GCRDGPQGIWGQDRCG, SEQ ID NO: 5). In some aspects, hydrogel microparticles can prepared using crosslinking HA with MMP dithiol crosslinkers.

In various aspects, the hydrogel microparticles are prepared using microfluidics (for example, droplet microfluidics) where hydrogel microparticles are formed as droplets. Methods of preparing hydrogel microparticles (e.g, microgels) are generally known by those of skill in the art.

In various aspects, contacting the azido-EV with the HMP functionalized with the strained alkyne comprises incubating the azido-EV with the hydrogel microparticle at an elevated temperature. In some aspects, the elevated temperature is higher than 20° C., higher than 25° C., or higher than 30° C., higher than 35° C., higher than 40° C., or higher than 45° C., or higher than 50° C. In some aspects, the elevated temperature is from about 25° C. to 60° C., from about 30° C. to about 55° C., from about 30° C. to about 50° C., from about 30° C. to about 45° C., from about 30° C. to about 40° C. In some aspects, the elevated temperature is 25° C., 26° C., 27° C., 28° C., 29° C., 30° C. C, 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C. In some aspects, the elevated temperature is from about 30° C. to about 40° C. (i.e., is 30° C. C, 31° C., 32° C., 33° C., 34° C., 35° C., 36° C., 37° C., 38° C., 39° C., or 40° C.). In some aspects, the elevated temperature is 37° C.

In various aspects, contacting the azido-EV with the HMP functionalized with the strained alkyne comprises incubating the azido-EV with the hydrogel microparticle at an elevated temperature for a period of time. For example, in some aspects, the method comprises incubating the azido-EV with the hydrogel microparticle at an elevated temperature for at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, or at least 30 minutes. In some aspects, the method comprises incubating the azido-EV with the hydrogel microparticle at an elevated temperature for at least 30 minutes. In some aspects, the method comprises incubating the azido-EV with the hydrogel microparticle at an elevated temperature for up to 30 minutes, up to 35 minutes, up to 40 minutes, up to 45 minutes, up to 50 minutes, up to 55 minutes, up to 60 minutes, up to 65 minutes, up to 70 minutes, up to 75 minutes, up to 80 minutes, up to 85 minutes, or up to 90 minutes. In some aspects, the method comprises incubating the azido-EV with the hydrogel microparticle at an elevated temperature for 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, or 90 minutes. In some aspects, the method comprises incubating the azido-EV with the hydrogel microparticle at an elevated temperature for about 30 minutes.

In some aspects, the method comprises forming a stable triazole linkage between the strained alkyne on the HMP (e.g., DBCO) and the azido sugar on the EV (e.g., N-azidoacetylmannosamine-tetraacylated (Ac4MannAz)). In various aspects, the reaction between the strained alkyne and the azido sugar is a biorthogonal reaction which refers to a chemical reaction that can occur inside living organisms without interfering with the natural biological processes or causing any harm to the cells. One of the key features of a bioorthogonal reaction is its selectivity. It should be specific to the biomolecule of interest and not react with other molecules present in the cell. Furthermore, it should be fast, efficient, and occur under mild conditions, such as neutral pH, low temperature, or in the presence of water.

In various aspects, the method of making an EV-HMP comprises making a plurality of EV-HMPs. In some aspects, this plurality of EV-HMPs is referred to herein as an EV-HMP suspension, and may optionally further comprise an inert carrier (i.e., water, buffer, saline or the like).

### (c) Methods of Making EV-MAPs

Various aspects of the present disclosure are directed to methods of making extracellular vesicle microporous annealed particle hydrogels.

In various aspects, the methods of making the EV-MAPs comprise contacting one or more EV-HMPs (e.g., the EV-HMP suspension above) with a crosslinker (e.g., a crosslinker comprising at least two azide groups like tetra-polyethylene glycol-azide).

In various aspects, the EV-HMPs are contacted with a crosslinker for a time sufficient for linkages to form between two or more EV-HMPs in the hydrogel. In some aspects, the EV-HMPs are contacted with the crosslinker for a time sufficient for linkages to form between at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the EV-HMPs in the hydrogel. In some aspects, the EV-HMPs are contacted with the crosslinker for a time sufficient for linkages to form between about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% of the EV-HMPs in the hydrogel. In some aspects, the EV-HMPs are contacted with the crosslinker for a time sufficient for linkages to form between 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the EV-HMPs in the composition.

In some aspects, the time sufficient to form linkages between or more EV-HMPs of the hydrogel is at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, or at least 120 minutes. In some aspects, the time sufficient to form linkages between or more EV-HMPs of the hydrogel is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some aspects, the time sufficient to form linkages between or more EV-HMPs of the hydrogel is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

The crosslinker can comprise a crosslinker comprising at least two azide groups. In some aspects, the crosslinker comprises tetra-polyethylene glycol-azide.

In various aspects, the method comprises contacting the EV-HMPs with the crosslinker in vitro or in situ or in vivo.

### (d) Methods of Treatment

Various aspects of the present disclosure are directed to methods of treating subjects in need thereof with the compositions provided herein. In some aspects, the methods herein comprise inducing and/or increasing angiogenesis and/or axonogenesis in a subject, inducing tissue repair in a subject, and/or treating a stroke in a subject.

In some aspects, the methods comprise inducing and/or increasing angiogenesis and/or axonogenesis in a subject. For example, the method can comprise inducing and/or increasing angiogenesis. As used herein, angiogenesis refers to increasing formation of blood vessels or associated structures such that blood flow is increased to an area. In further aspects, inducing and/or improving angiogenesis can comprise inducing and/or improving revascularization (e.g., in a tissue where loss of vasculature as occurred). In another example, the method can comprise inducing and/or increasing axonogensis in a subject. As used herein, axonogenesis refers to increasing neuronal growth (e.g., axons, dendrites) in an area. As described below, this angiogenesis (including revascularization) and/or axonogenesis may occur at or near a site of a stroke in a patient (i.e., in an infarct or peri-infarct region).

In some aspects, the methods comprise inducing tissue repair in a subject. In some aspects, the tissue can comprise brain tissue, and the method comprises inducing brain tissue repair. In some aspects, methods of inducing tissue repair comprises decreasing an area of damaged tissue by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% from a starting damaged tissue area. Methods of assessing tissue repair are known in the art and can comprise for example various imaging techniques, histological analyses, biomarker analyses, mechanical testing, functional assessments, as well as evaluation of patient reported outcomes.

In various aspects, the methods comprise treating a stroke in a patient. A stroke is an infarction in a brain brought about by occlusion or hemorrhage of a blood vessel. As such, strokes may be classified as “hemorrhagic” or “occlusive”, as described previously. Loss of blood flow to a region of the brain causes rapid tissue damage to the brain and nearby structures. The etiology of stroke can therefore cause immediate tissue damage (e.g., occurring within 24 hours of the occlusion or hemorrhage) at the stroke site (i.e., the infarct) and can also lead to long term damage as blood flow is not immediately restored and surrounding regions of the infarct also experience tissue damage. In any of the aspects of treating a stroke or treating tissue damage brought about by a stroke, the stroke may be hemorrhagic. Alternatively, in any of the aspects of treating a stroke or treating tissue damage brought about by a stroke, the stroke may be occlusive.

Accordingly, methods of treating a stroke in a patient can comprise reducing or preventing short-term tissue damage at the stroke site. As used herein, the stroke site is the site of occlusion/hemorrhage in the brain and can further comprise any region of the brain directly innervated by the blood vessel that is occluded or hemorrhaging. In some aspects, the stroke site is also called an “infarct region” or a “stroke core”. In some aspects, the stroke site, infarct region, and/or stroke core comprises any tissue damaged within 24 hours of the stroke event (e.g., occlusion/hemorrhage).

Likewise, methods of treating a stroke in a patient can comprise more long-term stroke treatments. For example, in some aspects, a method of treating a stroke in a patient can comprise promoting and/or inducing long-term recovery after the stroke. As used herein, “promoting and/or inducing long-term recovery” comprises any beneficial outcome that increases likelihood of long-term recovery of a stroke patient. This can include, but is not limited to, improving overall neural functionality of the patient and include for example both conscious neural responsiveness (e.g., cognitive, motor, somatosensory function) and unconscious neural responsiveness (reflexes, breathing, homeostasis, etc).

In order to treat the stroke (i.e., in the short-term or in the long-term as described above), the methods may comprise increasing angiogenesis and/or axogensis in the subject and/or inducing tissue repair (i.e., brain tissue repair) in the subject. As mentioned, a stroke incident can lead to brain damage and loss of neural function at distal sites to the site of occlusion or hemorrhage. So, methods of promoting and/or inducing long-term recovery after a stroke can comprise inducing tissue repair, increasing angiogenesis, and/or angiogenesis at these distal locations as well as long-term ongoing tissue repair, angiogenesis and/or angiogenesis at the stroke site.

In various aspects, the methods of treating a subject in need thereof comprise administering or delivering a pharmaceutical composition comprising one or more EV-HMPs or a pharmaceutical composition comprising an EV-MAP hydrogel, as provided above, to the subject.

In any of these aspects, the methods may comprise delivering a pharmaceutical composition comprising one or more EV-HMPs to the subject and further administering a crosslinker to the subject. In various aspects, the pharmaceutical composition comprising one or more EV-HMPs may comprise two or more EV-HMPs. In these aspects, separate delivery of the crosslinker results in formation of the EV-MAP hydrogel in situ. In accord with these aspects, the crosslinker may be delivered before the pharmaceutical composition comprising one or more EV-HMPs, it may be delivered after the pharmaceutical composition comprising one or more EV-HMPs, or it may be delivered simultaneously with the pharmaceutical composition comprising one or more EV-HMPs. When delivered simultaneously, it is understood that the pharmaceutical composition comprising the one or more EV-HMPs is separate and distinct (i.e., is part of a different composition) than the crosslinking composition. In some aspects, the crosslinker is administered 1 to 10 days after administration of the pharmaceutical composition comprising one or more EV-HMPs. For example, in some aspects, the crosslinker is administered about 1 day, about 2 days, about 3 days, about 4 days, about 5 days, about 6 days, about 7 days, about 8 days, about 9 days, or about 10 days after administration of the pharmaceutical composition comprising one or more EV-HMPs. In some aspects, the crosslinker is administered about 5 days after administration of the pharmaceutical composition comprising one or more EV-HMPs. Suitable crosslinkers that may be delivered or administered according to these methods are described above and may include, for example, any crosslinker having more than two azide groups. In some aspects, the crosslinker that may be delivered comprises tetra-arm PEG-azide.

In any of the foregoing methods, the pharmaceutical composition comprising one or more EV-HMPs, the pharmaceutical composition comprising an EV-MAP hydrogel, and/or the crosslinker may be delivered to a brain of the subject.

In any of the foregoing methods, the pharmaceutical composition comprising one or more EV-HMPs, the pharmaceutical composition comprising an EV-MAP hydrogel, and/or the crosslinker may be delivered via stereotaxic injection. In some aspects, the pharmaceutical composition comprising one or more EV-HMPs, the pharmaceutical composition comprising an EV-MAP hydrogel, and/or the crosslinker may be delivered via stereotaxic injection into the brain of the subject. In some aspects, the pharmaceutical composition comprising one or more EV-HMPs, the pharmaceutical composition comprising an EV-MAP hydrogel, and/or the crosslinker may be delivered via stereotaxic injection into a stroke core of the subject.

In any of the foregoing methods, the pharmaceutical composition comprising one or more EV-HMPs, the pharmaceutical composition comprising an EV-MAP hydrogel, and/or the crosslinker may be delivered intravenously, intrathecally, intraventricularly, via direct infarct injection, via peri-infarct injection, or any combination thereof.

In any of the foregoing methods, particularly in methods directed to treating a stroke in a subject, the pharmaceutical composition comprising one or more EV-HMPs, the pharmaceutical composition comprising an EV-MAP hydrogel, and/or the crosslinker may be delivered directly into the stroke core (as defined above). For example, the pharmaceutical composition comprising one or more EV-HMPs, the pharmaceutical composition comprising an EV-MAP hydrogel, and/or the crosslinker may be delivered via direct infarct injection and/or peri-infarct injection.

In any of the foregoing methods, when the method comprises delivering the pharmaceutical composition comprising one or more EV-HMPs and the crosslinker to the subject, the crosslinker may be administered via the same route of administration of the pharmaceutical composition comprising one or more EV-HMPs. For example, both the pharmaceutical composition comprising one or more EV-HMPs and the crosslinker may be administered via stereotaxic injection into a stroke core of a subject. For example, both the pharmaceutical composition comprising one or more EV-HMPs and the crosslinker may be administered via direct infarct injection and/or peri-infarct injection.

In various aspects, a subject in need thereof may be a subject that has or is suspected of having a brain injury. In some aspects, the brain injury comprises or is caused by a traumatic injury (e.g., external injury), an aneurysm, a stroke (e.g., a hemorrhagic stroke or occlusive stroke), an infection, a cancer, or a combination of any thereof. In some aspects, the subject in need thereof has suffered or is suspected of suffering from a stroke.

In various aspects, the subject may be a human or nonhuman animal. In some aspects, the nonhuman animal is a vertebrate and may comprise a mammal or a non-mammal (e.g., nonhuman primates, sheep, dog, cat, horse, cow, chickens, amphibians, reptiles, and the like). In some aspects, the nonhuman animal is a companion animal (e.g., a dog, cat, horse, rabbit, or any other animal typically in close contact with a human). In some aspects, the nonhuman animal is a laboratory animal (e.g., a mouse, rat, or any other animal typically used in a scientific laboratory). In some aspects, the subject is a human or a mouse.

### IV. Kits

The present disclosure further provides kits comprising the compositions provided herein and for carrying out the subject methods as provided herein. For example, in one embodiment, a subject kit may comprise, consist of, or consist essentially of one or more of the following: (i) one or more EV-HMPs as provided herein; (ii) an EV-MAP hydrogel as provided herein; and/or (iii) pharmaceutical compositions as provided herein.

In some aspects, the subject kit comprises (i) one or more EV-HMPs or a pharmaceutical composition thereof and (ii) a crosslinker or a pharmaceutical composition comprising the crosslinker. Suitable crosslinkers may comprise any crosslinker capable of linking one or more EV-HMPs as described above. For example, the crosslinker can comprise a crosslinker having two or more azide groups (e.g., tetra-polyethylene glycol-azide).

In other embodiments, a kit may further include other components. Such components may be provided individually or in combinations and may provide in any suitable container such as a vial, a bottle, or a tube. Examples of such components include but are not limited to (i) one or more additional reagents, such as one or more dilution buffers; one or more reconstitution solutions; one or more wash buffers; one or more storage buffers, one or more control reagents and the like, (ii) one or more reagents for in vitro crosslinking of the of the HMP molecules provided herein; and the like (e.g., tetra-polyethylene glycol-azide). Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### EXAMPLES

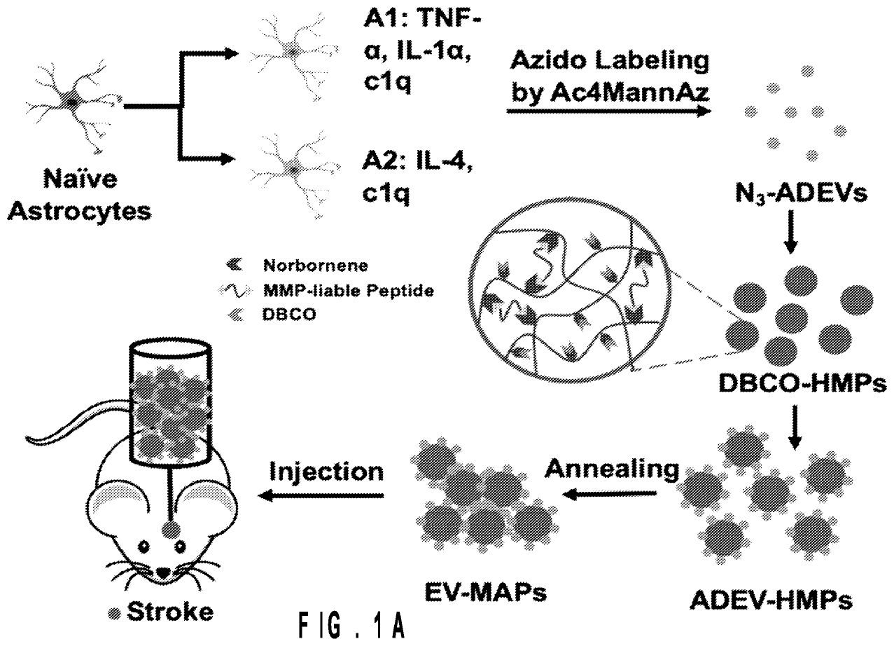

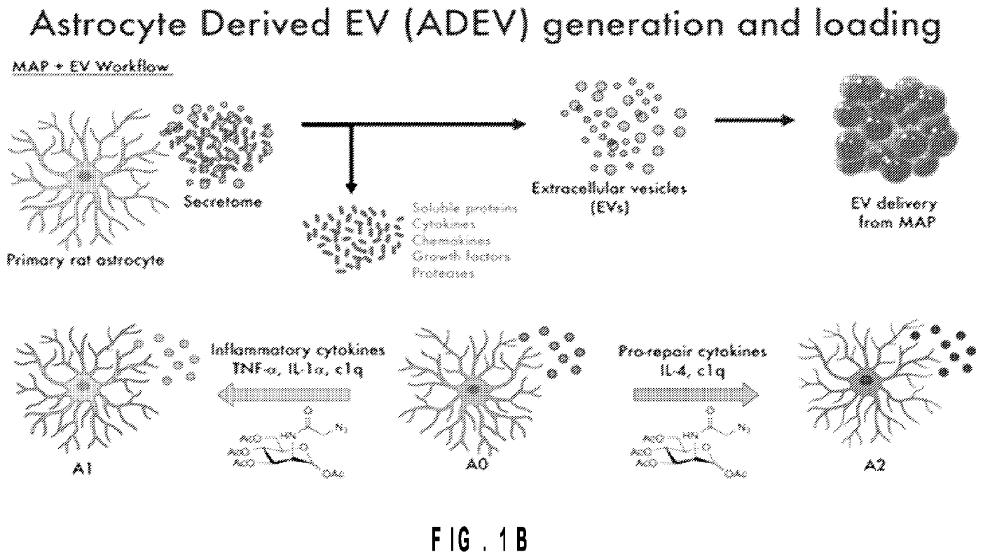

The following examples describe experiments using soluble activation of primary astrocytes or T cells extracted from rat pup cortices or liver to produce astrocytes with distinct gene expression profiles, collection of EVs from these astrocytes and analysis of their ability to promote repair after stroke in both peri-infarct and infarct areas (see e.g., FIG. 1A and FIG. 1B). Specifically, the following examples show that various derived EVs drive substantial regeneration of blood vessels and axons in the infarct after stroke and result in functional behavior recovery. The regeneration only occurs when Rep-EVs are immobilized onto a biomaterial scaffold. Further, the scaffold described here (e.g., MAPS) preserves the injectability of EVs after immobilization and the unique inner microporous structure present the infiltrating cells an environment of interconnected channels coated with astrocytic EVs. The presence of astrocytic or T-cell EVs enhances matrix remodeling by the infiltrating cells and transits the infarct from an inflammatory to a reparative environment as the major population of infiltrating microglia decreases significantly. Astrocytic or T-cell EVs also enrich pro-reparative proteins that are related to signaling pathways for tissue regeneration and functional recovery.

In summary, the following examples describe how a versatile biomaterial platform may be used to deliver EVs from various cell types for tissue repair.

### Example 1: Preparation of Astrocyte-Derived Extracellular Vesicles (ADEVs) from Naïve and Activated Astrocytes

In this example, astrocyte-derived extracellular vesicles (ADEVs) were isolated from activated and naïve rat cortical astrocytes. Specifically, using the following protocols, three sets of ADEVs were generated from (a) untreated rat primary astrocytes (untreated EV), (b) astrocytes treated with IL-1α, TNF-α, and C1q cytokine cocktail to obtain neurotoxic astrocytes (e.g., NTx-EV) and (c) astrocytes treated with interleukin-4 (IL-4) and C1q as an analog to the induction of pro-reparative macrophages (Rep-EV). The cocktail for (b) was chosen because it has been shown that activation of astrocytes with interleukin-1 alpha (IL-1α), tumor necrosis factor alpha (TNF-α), and the classical complement component C1q in vitro results in similar transcriptome profiles as compared to astrocytes activated with LPS in vivo (see e.g., Liddelow, S. A. et al. Nature 541, 481-487 (2017), which is incorporated herein by reference in its entirety). Therefore, this cocktail was used to obtain more established IL-1α/TNF-α/C1q induced astrocytes (referred to herein as NTx-EVs). On the other hand, while astrocytes isolated from middle cerebral artery occlusion (MCAO) stroke have shown pro-reparative gene profile (see e.g., Zamanian, J. L. et al. Journal of Neuroscience 32, 6391-6410 (2012), incorporated herein by reference in its entirety) there are no published reports of an in vitro reparative astrocyte cocktail. Therefore, the cocktail for (c) (including IL-4 and C1q) was chosen to generate reactive astrocytes with a potentially distinct phenotype compared to the IL-1α/TNF-α/C1q induced astrocytes based on a rationale that C1q has induced up-regulation of many MCAO-specific genes in combination with IL-1β, macrophages activate astrocytes and that reparative macrophages that promote tissue repair secrete IL-4 (see e.g., Liddelow et al., supra).

First, rat cortical astrocytes were prepared from PI rat cortices as described in Stogsdill, J. A. et al. Nature 551, 192-197 (2017), which is incorporated herein by reference in its entirety. Briefly the cortices were microdissected and papain digested followed by trituration in low and high ovomucoid solutions. Cells were passed through a 20 μm mesh filter, resuspended in astrocyte growth media (AGM; DMEM, 10% FBS, 10 μM hydrocortisone, 100 U/mL Pen/Strep, 2 mM L-Glutamine, 5 μg/ml Insulin, 1 mM NaPyruvate, 5 μg/ml N-Acetyl-L-cysteine) and 15-20 million cells were plated on 75 mm2 flasks (non-ventilated cap) coated with poly-D-lysine. Flasks containing cells were incubated at 37° C. in 5% CO2. On DIV3, AGM was removed and replaced with DPBS. Flasks were then shaken by hand for 10-15 times until only the adherent monolayer of astroglia remained. DPBS was then replaced with fresh AGM. Cytosine arabinoside (AraC) was supplemented to the media on DIV5 for two days to eliminate fast dividing cells.

Second, prepared astrocytes were then left untreated or treated with one of two different inducing cytokine cocktails before extracellular vesicles were isolated. Specifically, on DIV6, AGM were replaced by EV-collecting media (DMEM, 10% exosome-depleted FBS, 10 μM hydrocortisone, 100 U/mL Pen/Strep, 2 mM L-Glutamine, 5 μg/mL Insulin, 1 mM NaPyruvate, 5 μg/ml N-Acetyl-L-cysteine, 50 μM Ac4ManNAz) with the inducing cytokines (3 ng/ml Il-1α, 30 ng/ml TNF, and 400 ng/ml C1q for A1 activation, 20 ng/mL IL-4 and 400 ng/ml C1q for A2 activation). After 24 hrs, the culture media were collected and centrifuged at 2000 g for 30 minutes to remove cells and debris. The supernatant was transferred into a new tube and mixed with half volume of the total exosome isolation kit (Thermo Scientific) for overnight incubation at 4° C. The solutions were then centrifuged at 10,000 g for 60 mins at 4° C. and the resulted ADEV pellets were ready to be resuspended in PBS for downstream process.

The quantity and size of ADEVs were characterized using nanoparticle tracking analysis. EVs collected from NTx, Rep, and UT astrocytes showed multiple size vesicles with the major size peaks between 50-150 nm, the exosome size range (FIG. 1B). The total astrocyte derived (AD) EV concentration was the same (107 particles/mL) for all treatment conditions, indicating the cytokine treatment did not influence astrocyte derived EV secretion.

### Example 2: Genomic Profiles of Untreated, NTx and Pro-Reparative Astrocytes were Analyzed Using RNA Sequencing

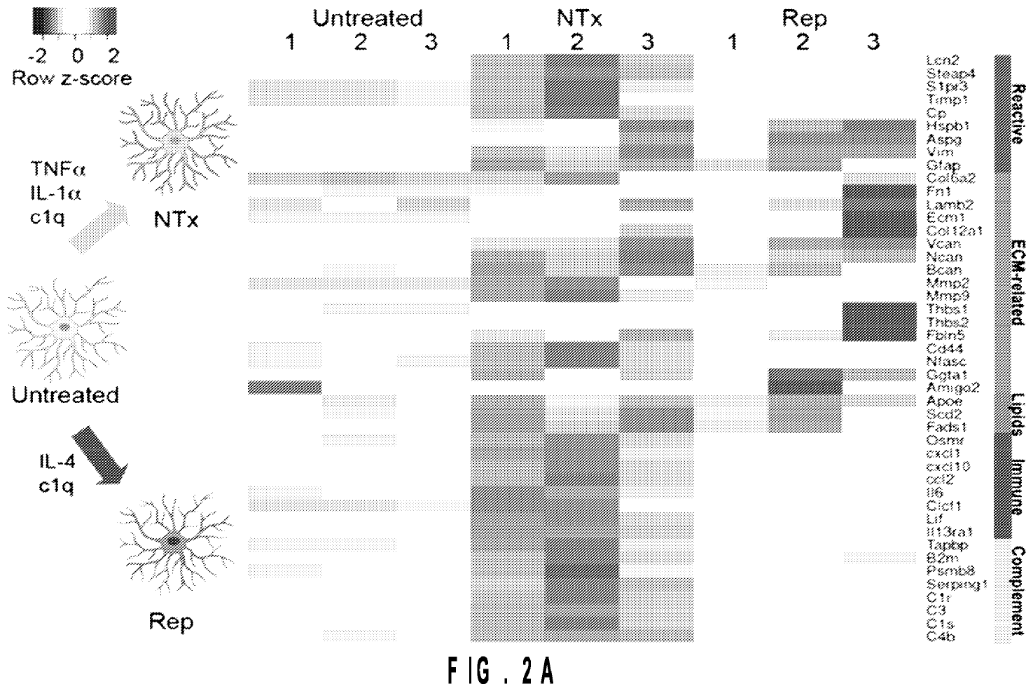

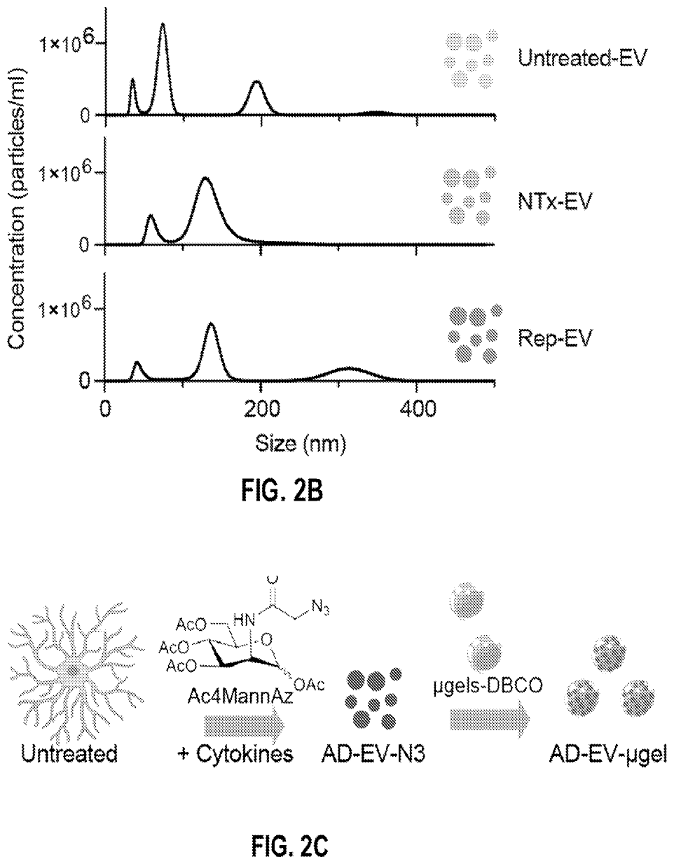

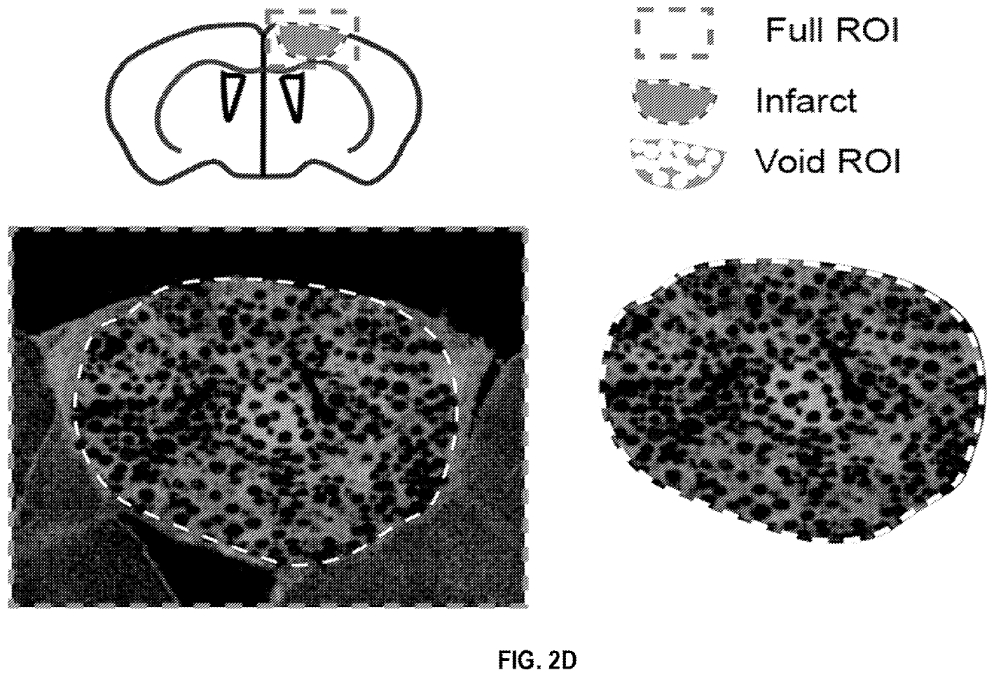

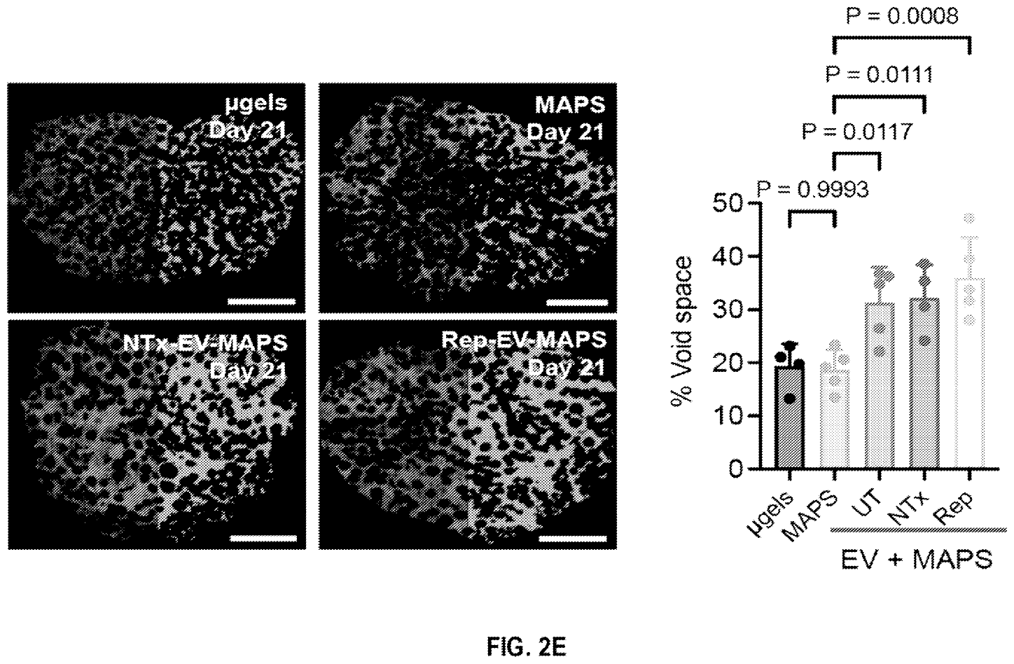

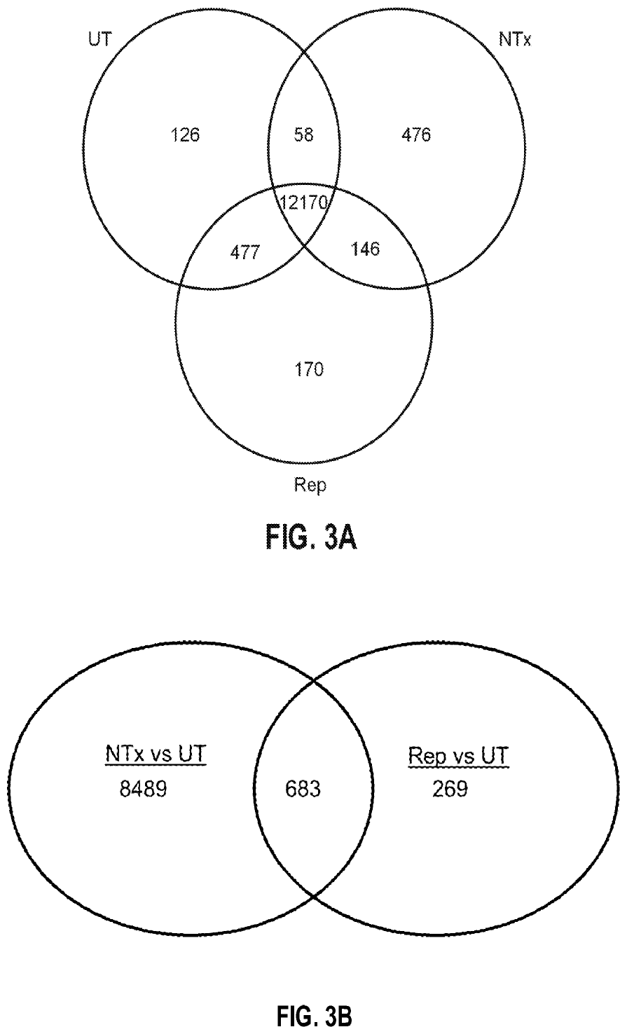

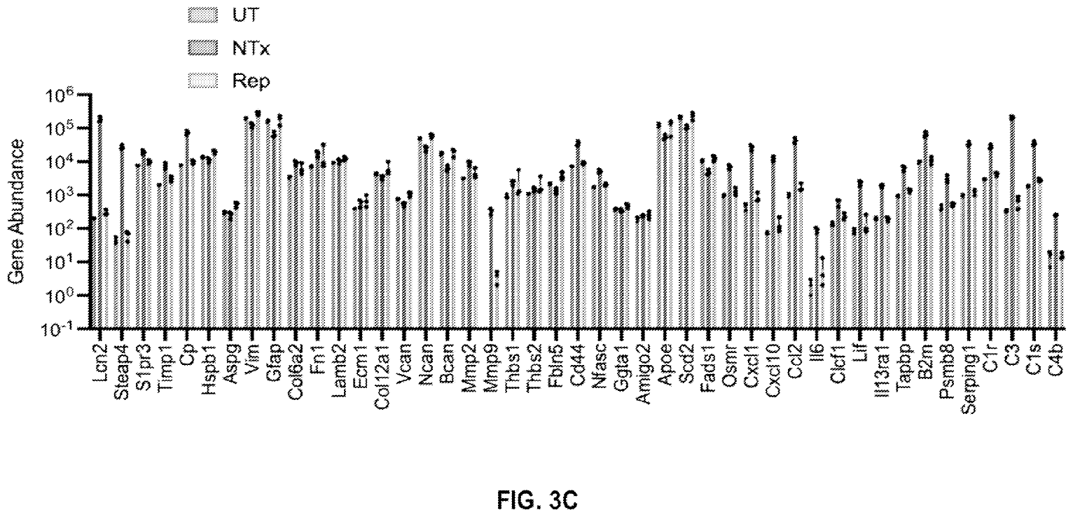

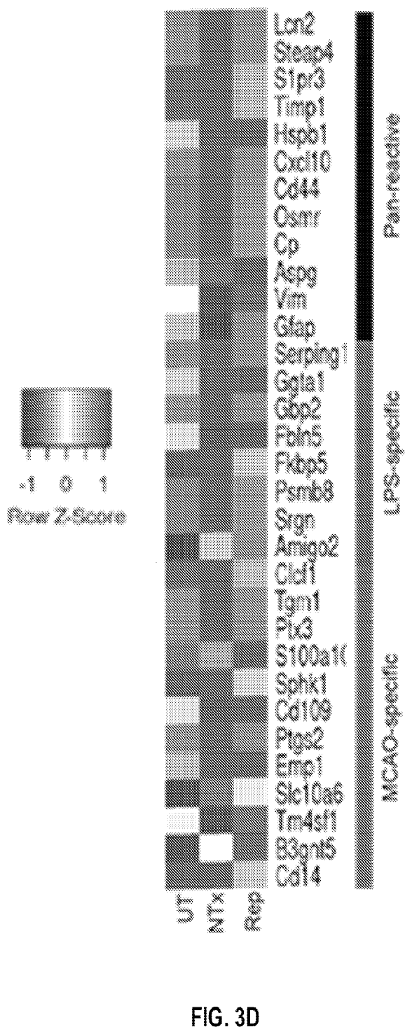

To verify the genomic phenotypes of the untreated (UT), IL-1α/TNF-α/C1q, and IL-4/C1q induced astrocytes, we first investigated their transcriptome by RNA sequencing using standard protocols (FIG. 2A and FIG. 3A-FIG. 3C). 13,176 genes were detected from these samples and 92.36% of them overlapped among the three astrocytes (FIG. 3A and FIG. 3B). IL-1α/TNF-α/C1q induced astrocytes showed significant up-regulation of Lcn2, Cp, and Steap4, indicating they became strongly reactive, and they had up-regulation of some ECM genes (Col6a2, Fn1), several matrix metalloproteinases, proteins involved in cell adhesion (CD44, Nfasc), genes related to immune responses (Osmr, Cxcl10, Il6) and complement pathways (C3, Serping1) (FIG. 2A and FIG. 3C). For ease of reference, relative expression of genes visually depicted in FIG. 2A is described in Table 1 herein below.

TABLE 1

| | | Relative Expression ^{A} | | | | | | | | |
|---|---|---|---|---|---|---|---|---|---|---|
| | | UT* | UT | UT | Ntx* | Ntx | Ntx | Rep* | Rep | Rep |
| Gene | Group | Grp 1 | Grp 2 | Grp 3 | Grp 1 | Grp 2 | Grp 3 | Grp 1 | Grp 2 | Grp 3 |
| Lcn2 | Reactive | Down | Down | Down | Up | Up | Up | Down | Down | Down |
| Steap4 | Reactive | Down | Down | Down | Up | Up | Up | Down | Down | Down |
| S1pr3 | Reactive | Down | Down | Down | Down | Down | Down | Down | Down | NC |
| Timp1 | Reactive | Down | Down | Down | Up | Up | Up | Down | Down | NC |
| Cp | Reactive | Down | Down | Down | Up | Up | Up | Down | Down | Down |
| Hspb1 | Reactive | Down | NC | NC | Down | Down | Down | Up | Up | Up |
| Aspg | Reactive | NC | Down | NC | Down | Down | Down | Up | Up | Up |
| Vim | Reactive | NC | NC | NC | Down | Down | Down | Up | Up | Up |
| Gfap | Reactive | NC | Up | Up | Down | Down | Down | Up | Up | NC |
| Col6a2 | ECM | Down | Down | Down | Up | Up | Up | Down | Up/NC | Up |
| Fn1 | ECM | Down | Down | Down | Up | Up | NC | Down | Down/NC | Up |
| Lamb2 | ECM | Down | Down | Down | NC | Up | Down | NC | Up | Up |
| Ecm1 | ECM | Down | Down | Down | NC | Up | Down | Down | Up/NC | Up |
| Col12a1 | ECM | NC | NC | NC | Down | Down | Down | NC | NC | Up |
| Vcan | ECM | NC | NC | NC | Down | Down | Down | NC | Up | Up |
| Ncan | ECM | NC | Up | NC | Down | Down | Down | Up | Up | Up |
| Bcan | ECM | NC | Up | Up | Down | Down | Down | Up | Up | NC |
| mmp2 | ECM | Down | Down | Down | Up | Up | Up | Down | Down | NC |
| mmp9 | ECM | Down | Down | Down | Up | Up | Up | Down | Down | Down |
| Thbs1 | ECM | Down | Down | Down | NC | NC | NC | Down | Down | Up |
| Thbs2 | ECM | Down | Down | Down | NC | NC | NC | NC | NC | Up |
| Fbln5 | ECM | NC | NC | NC | Down | Down | Down | NC | Up | Up |
| Cd44 | ECM | Down | Down | Down | Up | Up | Up | Down | Down | Down |
| Nfasc | ECM | Down | Down | Down | Up | Up | Up | Down | Down | Down |
| Ggta1 | ECM | Down | NC | NC | Down | Down | Down | NC | Up | Up |
| Amigo2 | ECM | Down | Down | Down/NC | NC | NC/Up | NC | Down/NC | Up | Down |
| Apoe | Lipids | NC | Up | Up | Down | Down | Down | Up | Up | Down |
| Scd2 | Lipids | NC | Up | Up | Down | Down | Down | Up | Up | NC |
| Fads1 | Lipids | NC | Up | Up | Down | Down | Down | Up | Up | NC |
| Osmr | Immune | Down | Down | Down | Up | Up | Up | Down | Down | Down |
| Cxcl1 | Immune | Down | Down | Down | Up | Up | Up | Down | Down | Down |
| Cxcl10 | Immune | Down | Down | Down | Up | Up | Up | Down | Down | Down |
| ccl2 | Immune | Down | Down | Down | Up | Up | Up | Down | Down | Down |
| Il6 | Immune | Down | Down | Down | Up | Up | Up | Down | Down | Down |
| Clcf1 | Immune | Down | Down | Down | Up | Up | Up | Down | Down | NC |
| Lif | Immune | Down | Down | Down | Up | Up | Up | Down | Down | Down |
| Il13ra1 | Immune | Down | Down | Down | Up | Up | Up | Down | Down | Down |
| Tapbp | Complement | Down | Down | Down | Up | Up | Up | Down | Down | Down |
| B2m | Complement | Down | Down | Down | Up | Up | Up | Down | Down | Down |
| Psmb8 | Complement | Down | Down | Down | Up | Up | Up | Down | Down | Down |
| Serping1 | Complement | Down | Down | Down | Up | Up | Up | Down | Down | Down |
| C1r | Complement | Down | Down | Down | Up | Up | Up | Down | Down | Down |
| C3 | Complement | Down | Down | Down | Up | Up | Up | Down | Down | Down |
| C1s | Complement | Down | Down | Down | Up | Up | Up | Down | Down | Down |
| C4b | Complement | Down | Down | Down | Up | Up | Up | Down | Down | Down |

^{A} Relative Expression is defined as “Down” if Z score is between −2 and 0; as “Up” if Z score is between 0 and +2, and as “NC” if it is ~0.
*UT = untreated; Ntx = Neurotoxic; Rep = Reparative as described herein.

FIG. 3D shows that, shows that the IL-1α/TNF-α/C1q induced astrocytes had different expression profiles compared to neurotoxic A1 astrocytes reported in Liddelow et al., Nature 541, 481-487 (2017), such as down-regulation of some pan-reactive markers (Vim, Gfap), fatty acid related markers (Apoe, Scd2), and LPS-specific markers (Ggta1, Fbln5). Also, there are several MACO-specific genes up-regulated in our IL-1α/TNF-α/C1q induced astrocytes (Clcf1, Tgm1, Ptx3, Sphk1, Ptgs2, Cd14). In contrast, IL-4/C1q induced astrocytes up-regulated pan-reactive markers (Hspb1, Aspg, Vim, Gfap) and some MACO-specific genes (S100a10, Cd109, Emp1, Tm4sf1, B3gnt5). They also had notable up-regulation of some ECM proteins, such as Col12a1, Ecm1, Vcan, Ncan, Bcan. For ease of reference, relative expression of genes visually depicted in FIG. 3D is described in Table 2 herein below.

TABLE 2

| | Relative Expression^{A} | | | | Relative Expression^{A} | | |
|---|---|---|---|---|---|---|---|
| Gene | UT* | NTx* | Rep* | Gene | UT* | NTx* | Rep* |
| Lcn2 | Down | Up | Down | Fkbp5 | Down | Up | Down |
| Steap4 | Down | Up | Down | Psmb8 | Down | Up | Down |
| S1pr3 | Down | Up | Down | Srgn | Down | Up | Down |
| Timp1 | Down | Up | Down | Amigo2 | Down | Up | Up |
| Hspbl | Down | Down | Up | Clcf1 | Down | Up | Down |
| Cxcl10 | Down | Up | Down | Tgm1 | Down | Up | Down |
| Cd44 | Down | Up | Down | Ptx3 | Down | Up | Down |
| Osmr | Down | Up | Down | S100a1 | Down | Down | Up |
| Cp | Down | Up | Down | Sphk1 | Down | Up | Down |
| Aspg | Down | Down | Up | Cd109 | NC | Down | Up |
| Vim | NC | Down | Up | Ptgs2 | Down | Up | Down |
| Gfap | Up | Down | Up | Empl | Down | Down | Up |
| Serping1 | Down | Up | Down | Slc10a6 | Down | Up | NC |
| Ggta1 | Down | Down | Up | Tm4sf1 | NC | Down | Up |
| Gbp2 | Down | Up | Down | B3gnt5 | Down | NC | Up |
| Fbln5 | NC | Down | Up | Cd14 | Down | Up | Down |

^{A}Relative Expression is defined as “Down” if Z score is between −2 and 0; as “Up” if Z score is between 0 and +2 and as “NC” if it is about 0.
*UT = untreated; Ntx = Neurotoxic; Rep = Reparative as described herein.

Together, the transcriptome profiles of IL-1α/TNF-α/C1q, and IL-4/C1q activated UT astrocytes were distinct, allowing for testing whether reactive astrocytes release EV that promote recovery after stroke. Since the IL-1α/TNF-α/C1q induced astrocytes that had many reactive and inflammatory genes significantly up-regulated, these astrocytes produced from this cocktail were labeled as neurotoxic (NTx) astrocytes. IL-4/C1q induced astrocytes had fewer reactive genes up-regulated and showed strong up-regulation in many pro-reparative genes; and were labeled as reparative (Rep) astrocytes.

### Example 3: Astrocyte Derived Extracellular Vesicles (ADEVs) Did not Improve Tissue Regeneration after Stroke when Administered Alone

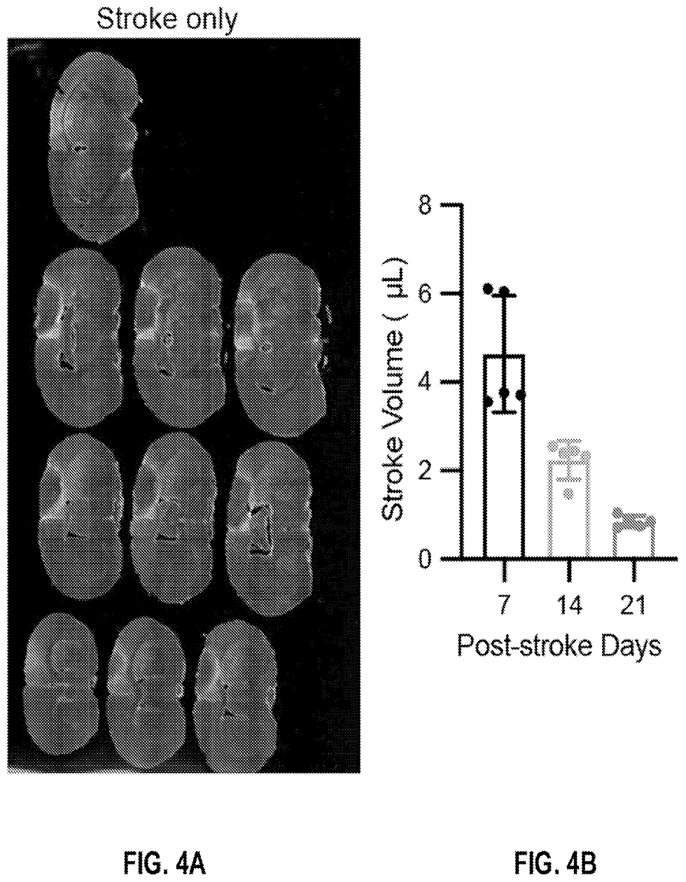

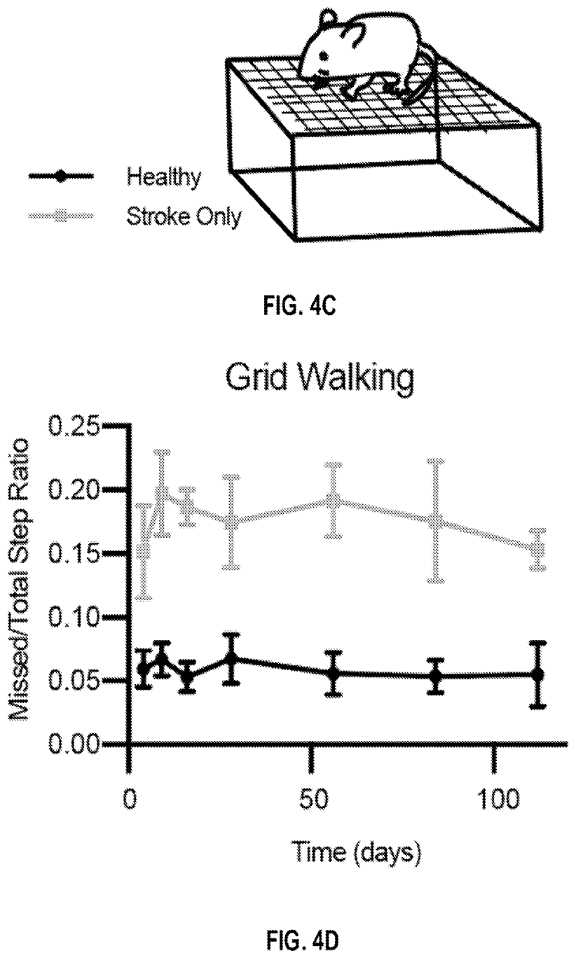

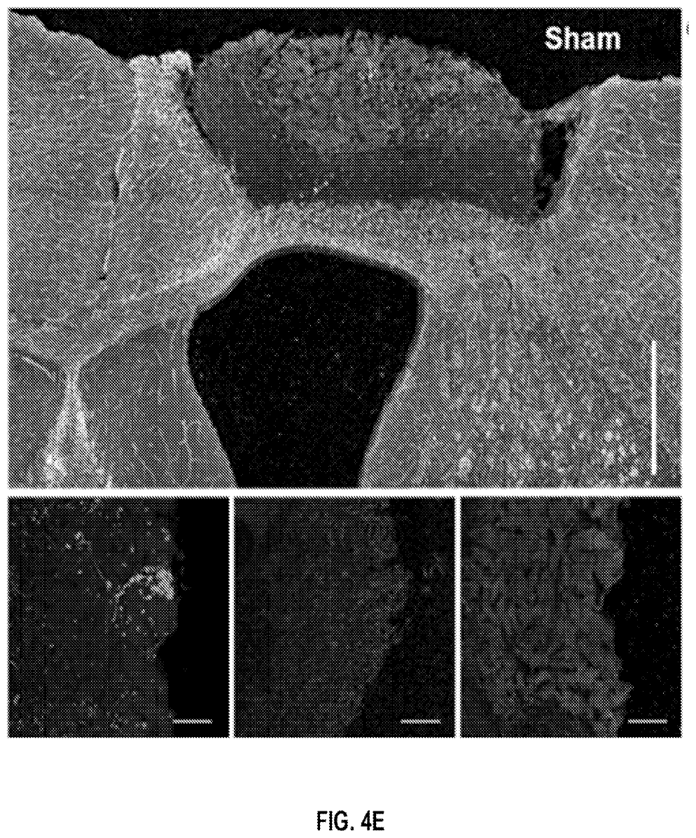

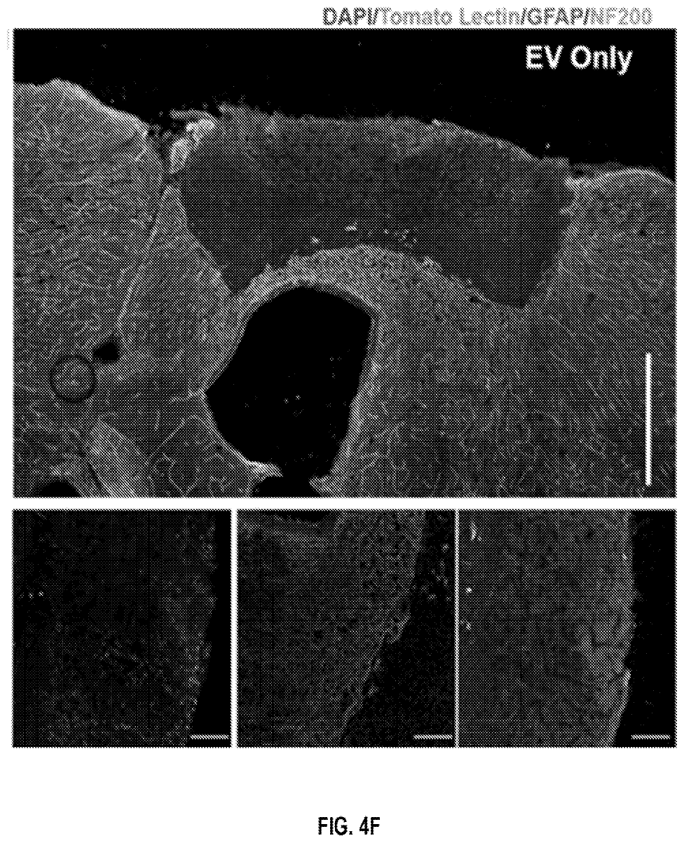

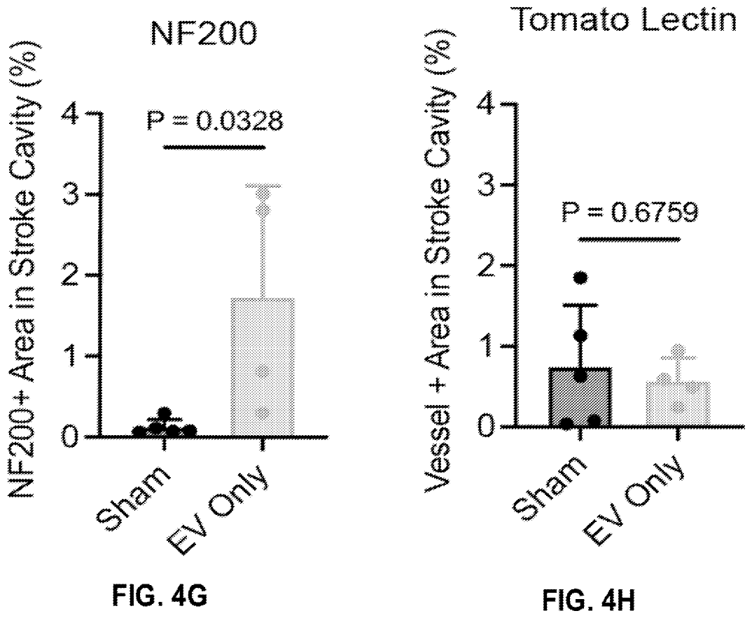

In this example, experiments are described showing that astrocyte-derived extracellular vesicles cannot improve tissue regeneration on their own. A mouse protothrombotic stroke model was used where PT stroke was induced using an intraperitoneal (i.p.)-injected light-sensitive dye. Briefly, mice were positioned in a stereotaxic instrument and administered Rose Bengal (10 mg/ml, 10 uL/g i.p.). After 7 minutes, a laser was illuminated on the spot of the closed skull 1.5 mm left from the bregma for 13 minutes. 5 days post-stroke surgery 2×10⁶ A1 or A2 ADEVs in 6 μL PBS buffer were injected into the stroke cavity via a 30-gauge needle attached to a 10 μl Hamilton syringe at 1 μl/min. The needle was positioned at 0.75 mm penetration from the skull. The control groups were injected with PBS. Brain tissues were collected at Day 21. FIG. 4A-4D show that stroke in these studies spanned all cortex layers (FIGS. 4A-4B) and resulted in a significant motor deficit in the left limb of the mouse (FIGS. 4C-4D).

To assess therapeutic potential of the EVS alone, Rep-EV (1.8×10⁶ particles/mL) were injected directly into the stroke cavity as described in Nih, L et al., Nature materials 17, 642-651 (2018) and Sideris, E. et al. Advanced Therapeutics, 2200048 (2019), which are incorporated herein by reference in their entirety. As described therein, therapeutic agents are injected into the infarct at Day 5 post-stroke, a timepoint at which a cavity has formed and pro-repair pathways remain active. Rep-EV injection (16-days post injection) yielded similar outcomes in the infarct as the sham, no treatment group (FIG. 4E-4F, with quantification in FIG. 4G). Rep-EV injection resulted a slight improvement of axon density in the peri-infarct region (FIG. 4H).

### Example 4: Preparation of ADEV-HMPs and ADEV-MAP Hydrogels

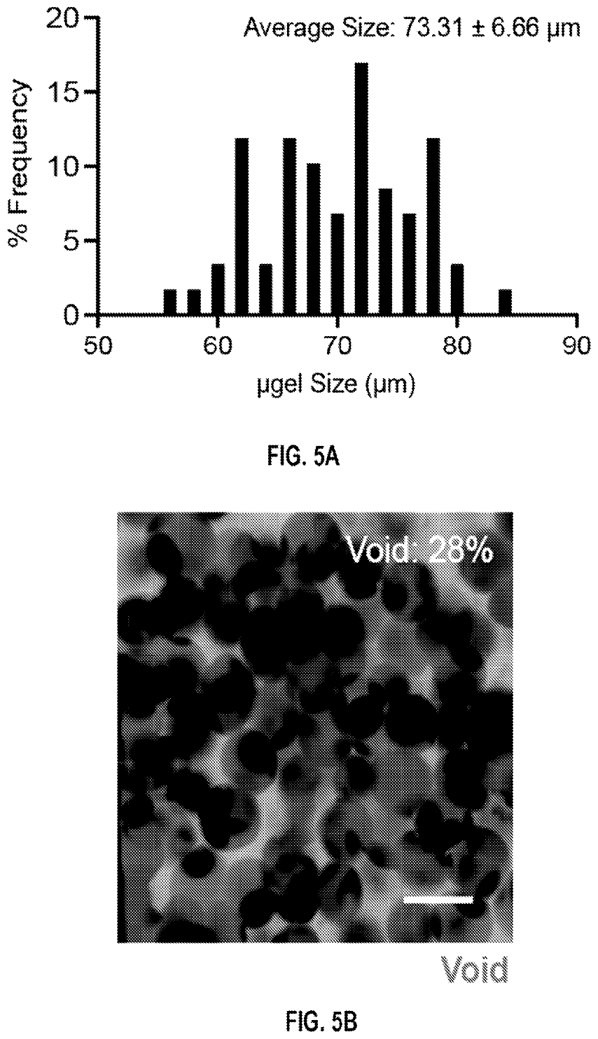

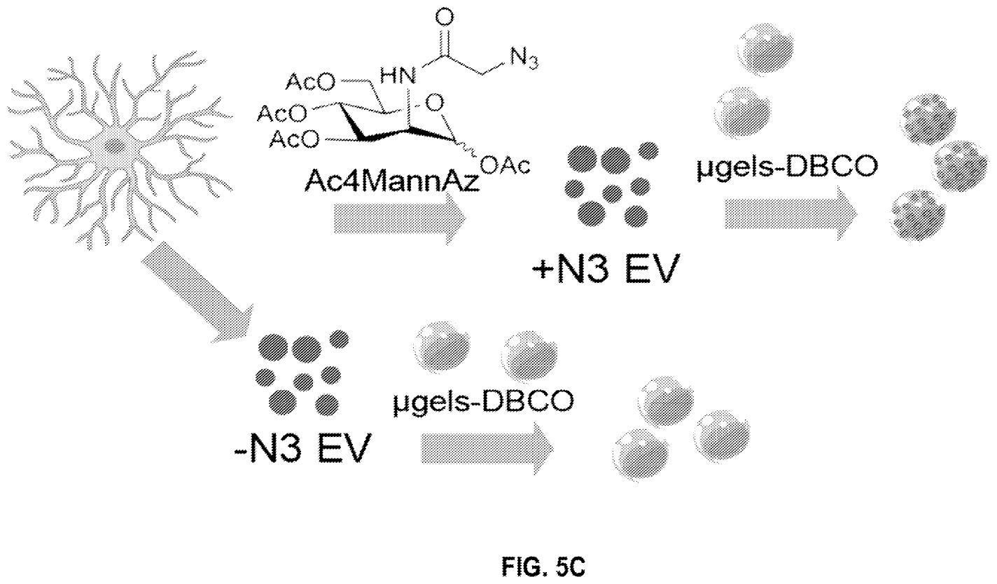

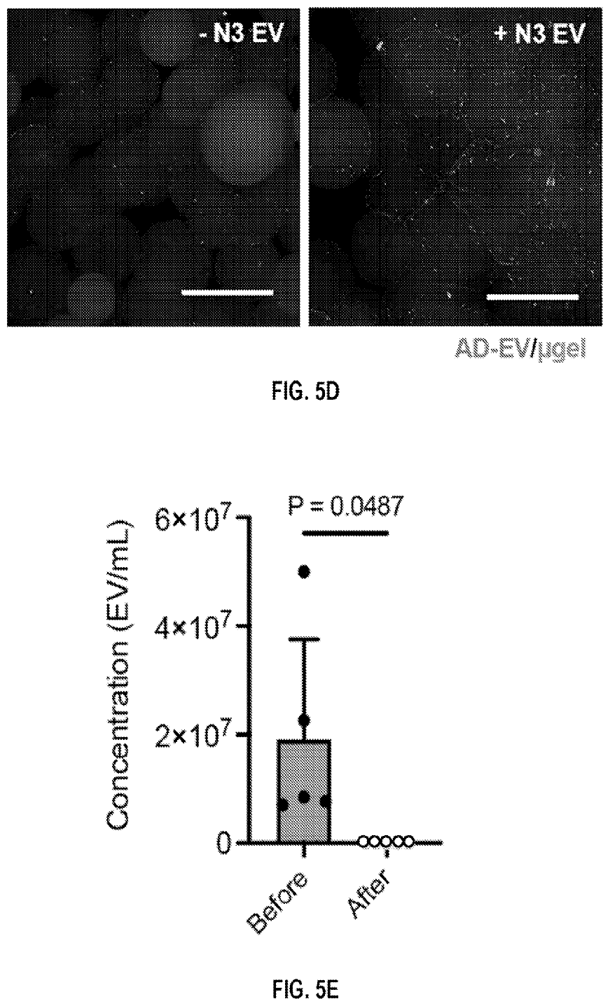

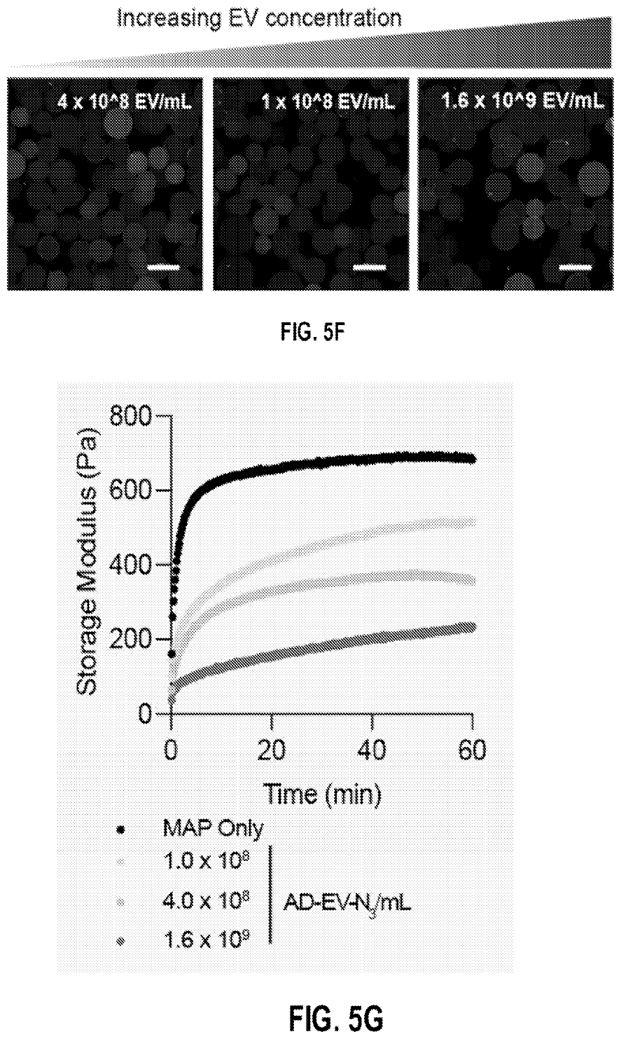

This example describes how ADEVs are immobilized onto HMPs and then prepared into ADEV-microporous annealed particle (ADEV-MAP) hydrogels or scaffolds, in vitro and in situ. The ADEV-MAP hydrogels comprise a defined matrix scaffold (microporous annealed particle scaffold or MAPS) composed of hyaluronic acid microgel building blocks, which can undergo gelation in situ through a bio-orthogonal strain-promoted azide-alkyne click reaction to form a porous hydrogel that allows for diffusion of soluble factors and cell infiltration. MAPS are fundamentally different than other conventional hydrogels because they are granular materials, injectable and porous, rather than continuous polymeric networks that must degrade for cellular remodeling to take place. The hydrogels prepared in this example were also modified with a fibronectin derived RGD (Arg-Gly-Asp) peptide to create a chemically defined environment. As described further below, strain-alkyne bearing microgels with an average size of 73.31 μm were used, generating MAPS with 28% void space after in vitro assembled by multi-arm azide crosslinker. (FIG. 5A-5B)

First, astrocytic EVs collected in Example 1 were metabolically labeled as shown in FIG. 2C with the azido-sugar to form an “azido-ADEV suspension”. N-azidoacetylmannosamine-tetraacylated (Ac4MannAz) was chosen as the azido sugar because it has been previously shown to introduce azide groups at the glycocalyx of the EVs (see e.g., Lee, T. S et al., Biochimica et Biophysica Acta (BBA)-General Subjects 1862, 1091-1100 (2018) and Baskin, J. M. et al. Copper-free click chemistry for dynamic in vivo imaging. Proceedings of the National Academy of Sciences 104, 16793-16797 (2007), which are incorporated herein by reference in their entirety. At the same time, “DBCO-labeled HMPs” were prepared using microfluidic device was made of polydimethylsiloxane (PDMS) by standard soft lithography. Master molds were fabricated on a 4-inch silicon wafer by a photolithographic technique using a negative photoresistor (SU8 2075). Microfluidic devices were molded from master molds by pouring degassed PDMS (elastomer:crosslinker=10:1) and cured at 60° C. for 1 h. PDMS devices were then placed onto a glass slide and bonded together at 60° C. overnight. The bonded device was treated with Rain-X to render a hydrophobic surface. HMP droplets were generated at the flow focusing region where the oil phase broke off the gel solution into droplets. The gel solutions consisted of 3.4 wt % hyaluronic acid-norbornene (MW=79 kDa, degree of functionality was either 28% or 35%), MMP-degradable crosslinker GCRDGPQGIWGQDRCG (SEQ ID NO: 5, 2.3 mM), lithium phenyl-2,4,6-trimethylbenzoylphosphinate (10 mM), and RGDSPGERCG (SEQ ID NO: 2, 1 mM). The oil phase included heavy mineral oil with Span 80 (15 wt %). The flow rate for gel precursors was 0.3 L/min and that for oil phase was 2.7 uL/min. The HMP droplets were cured downstream with ultraviolet (UV) irradiation (20 mW/cm2, 365 nm). The prepared norbornene-bearing HMPs were washed and sterilized with PBS, 1% pluronic F127, and 30% ethanol. DBCO-tetrazine was used to convert the free norbornene groups to DBCO groups through Diels-Alder click reaction at 37° C. for 4 hours.

Once prepared, the DBCO-excessive HMPs were incubated with azido-ADEV suspension at 37° C. for 30 minutes to make ADEV-conjugated HMPs. ADEVs were fluorescently stained with plasma membrane dye to visualize their conjugation on microgels by confocal imaging. Conjugation specific, only astrocytic EVs that were metabolically labeled with azides resulted in nearly complete conjugation to strain-alkyne containing matrix (FIG. 5C) since virtually no EVs were observed in solution after conjugation (FIG. 5D-5E). It was also found that the amount loaded was dose dependent (FIG. 5F) and impacted the gelation kinetics and final bulk storage modulus of the hydrogel matrix. In situ monitoring of ADEV-MAP hydrogel gelation was performed on rheometer at 1% strain and 2.5 rads/s frequency. Increasing the concentration of astrocytic EVs resulted in more astrocytic EVs conjugated to the microgel and a decreased modulus because of reduced microparticle interlinking sites that were now occupied by EVs (FIG. 5G). Accordingly, 4×10⁸ EVs per milliliter of microgels were chosen for subsequent experiments as it allows for a significant number of EVs immobilization, while keeping the bulk modulus near that of the brain (300 Pa). This EV dose was also consistent with the EV only treatment in FIG. 4E (Example 3).

### Example 5: Astrocytic EVs Enhance MAP Scaffolds Degradation in Stroke Infarct

To evaluate the therapeutic outcomes of astrocytic EV+MAP on post stroke repair, 4.5 μL of astrocytic EV modified microgels (“ADEV-conjugated HMPs” from Example 4) along with a tetra-polyethylene glycol-azide crosslinker were injected into the stroke cavity (using a 30 gauge needle attached to a 10 μL Hamilton syringe delivering at 1 μL/min and positioned at 0.75 mm penetration from the skull) in a mouse photothrombotic stroke model at Day 5 post-stroke. Control animals were injected with PBS or the same amount of ADEVs in PBS solution (e.g., Example 1).

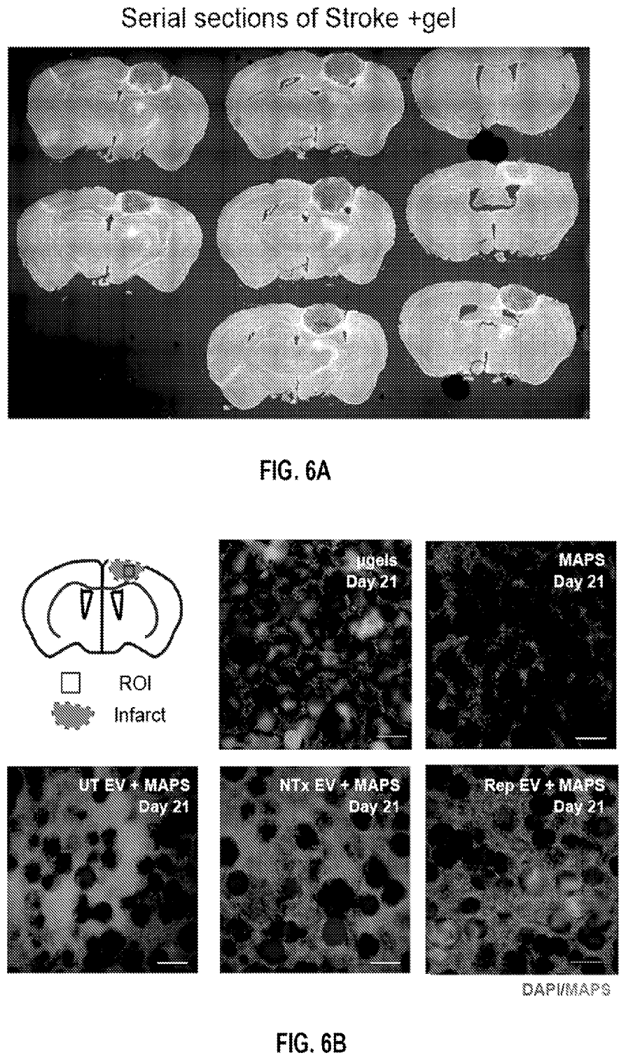

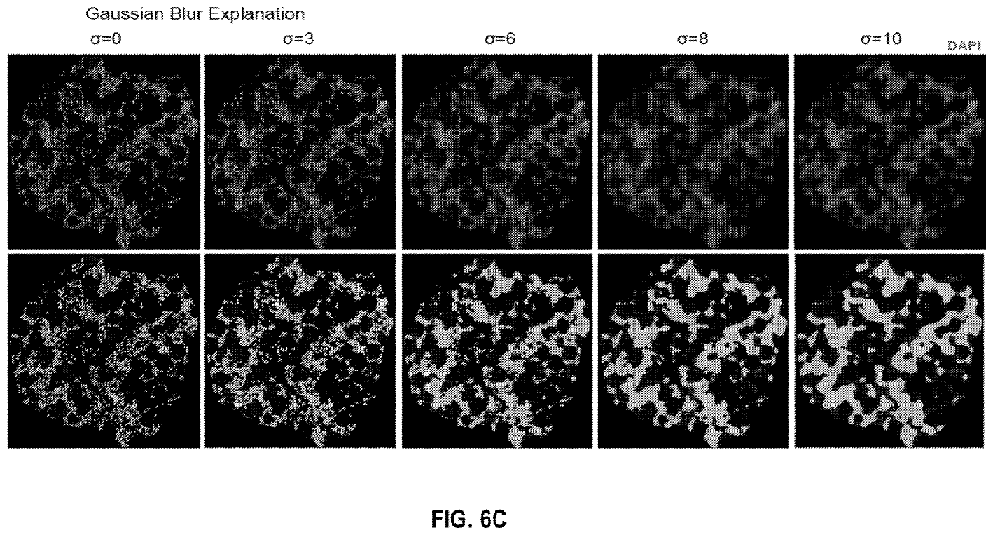

At Day 21 post-stroke, mice were retro-orbitally injected with biotinylated tomato lectin (0.1 mg) 10 mins before perfusion and then transcardially perfused with cold PBS and 4% paraformaldehyde. Brains were harvested and postfixed in 4% PFA for 2 hrs and immersed in 30% sucrose until sinking for cryoprotection. Tangential sectioning of frozen brains (30 μm thick) was obtained using a cryostat and mounted on gelatin-coated glass slides for confocal imaging and other analysis. Large confocal scans on the entire stroke infarct were performed by stitching 4*5 20X field-view (FIG. 6A). Brain sections from 900-1200 μm into the stroke infarct were used for tissue study quantification using the following methods.

MAPS scaffolds are infiltrated by stroke and peri-infarct resident cells through its void space, space between microgel particles, and not through direct microgel infiltration (e.g., see Sideris, E. et al. Advanced Therapeutics, 2200048 (2019). Accordingly, the DAPI signals of infiltrating cells within the ischemic core were used to quantify the void space of MAPS and EV+MAPS groups. This helped determine the degradation of MAPS and monitor hydrogel remodeling by cells over time. It was predicted that MAPS remodeling would result in more void which in turn would result in more DAPI area. In line with previous reports, it was observed that the void space within the implanted MAPS was filled by residing and infiltrating cells (FIG. 2D and FIG. 6B). To account for the cell body and matrix deposition, the DAPI signal was connected using a Gaussian Blur to estimate the degree of MAPS remodeling in the stroke environment over time (FIG. 6C). By applying this gaussian blur to the DAPI signal, a mask for tissue within the infarct area was created and the variance (σ) of a gaussian blur determines how much blur there is. This value was chosen as the one which obscured individual cells without removing features of the void (e.g., σ=8, FIG. 6C). This mask was then used to normalize area-based measurements made within the infarct.

As shown in FIG. 2E, NTx- and Rep-EV+MAP scaffolds (NTx, and Rep) as well as UT EVs displayed improved hydrogel degradation compared to the MAPS only group. This improved degradation was not caused by the reduced crosslinking linkages between microgels as results of astrocytic EV conjugation, because a microgel only (no interlinking) group did not have significant degradation. These results show that the delivery of astrocytic EVs on MAP gels results in improved matrix remodeling and cellular infiltration within the stroke infarct.

### Example 6: NTx- and Rep-EV-MAP Scaffolds Both Promote Angiogenesis after Stroke

Angiogenesis has been shown to be an essential component of biomaterial driven brain repair and behavioral improvement after ischemic stroke (see e.g., Nih, L. R et al., Nature materials 17, 642-651 (2018) which is incorporated herein by reference in its entirety. This example shows that both Ntx- and Rep-EV-MAP scaffolds promote angiogenesis after stroke.

Specifically, the effect of Ntx and Rep-EV-MAP scaffolds on angiogenesis was monitored by quantifying the density and morphology of perfused vessels that were connected to the circulation system after treatment with astrocytic EV decorated MAPS. Photothrombotic stroke model mice were treated with the MAP scaffolds and brain tissue obtained as described in Example 5. As described above, perfused vessels were labeled with tomato lectin before fixative perfusion, sectioning, and immunohistochemistry as described here. Specifically, immunohistochemistry included antigen-retrieval by incubating the tissue sections in citrate buffer pH=6 for 30 mins at 80° C., washing steps using PBS, and permeabilization/blockage using Triton (0.3%) and normal donkey serum (10%). Primary antibodies were as follows: rat anti-GFAP (1:400) for astrocytes; rabbit anti-Iba-1 (1:250) for microglias; rabbit anti-NF200 (1:200) for neurofilaments; goat anti-PDGFRβ (1:20) and rabbit anti-NG2 (1:500) for pericytes. Primary antibodies were incubated overnight at 4° C. followed by a fluorescently labelled secondary antibody (1:500) for 1 h at room temperature with streptavidin-conjugated dye (1:500) for biotinylated tomato lectin and DAPI (1:1000) for cell nuclei. After washing, Aquapoly was used as mounting media to mount coverslip onto the tissue sections.

Angiogenesis and axonogenesis were quantified by using tomato lectin and NF200 signal, respectively. Positive area was measured by denoising using the ImageJ Despeckle filter, and thresholding the image. Infiltration into the infarct was determined by taking the shortest distance between the edge of the ROI and signal within it. Blood vessels were further quantified by skeletonizing the tomato lectin signal and using the AnalyzeSkeleton plugin in ImageJ. To reduce noise, only vessels greater than 50 μm in length were considered. From the results, vessel average length, maximum length, number of branches, and count over area were calculated. For each sample, vessel thickness was the average of 20 random measurements of blood vessel cross-sections. The vessel intensity profile was also taken from cross-sections of blood vessels. PDGFRβ+ area surrounding the tomato lectin signal was measured by first selecting the vessels within a sample, enlarging the selection by 5 μm on each side, applying the selection onto the PDGFRβ image, and then thresholding appropriately. After thresholding NG2+ and PDGFRβ+ areas separately, the percent of the infarct positive for both stains was used for colocalization analysis.

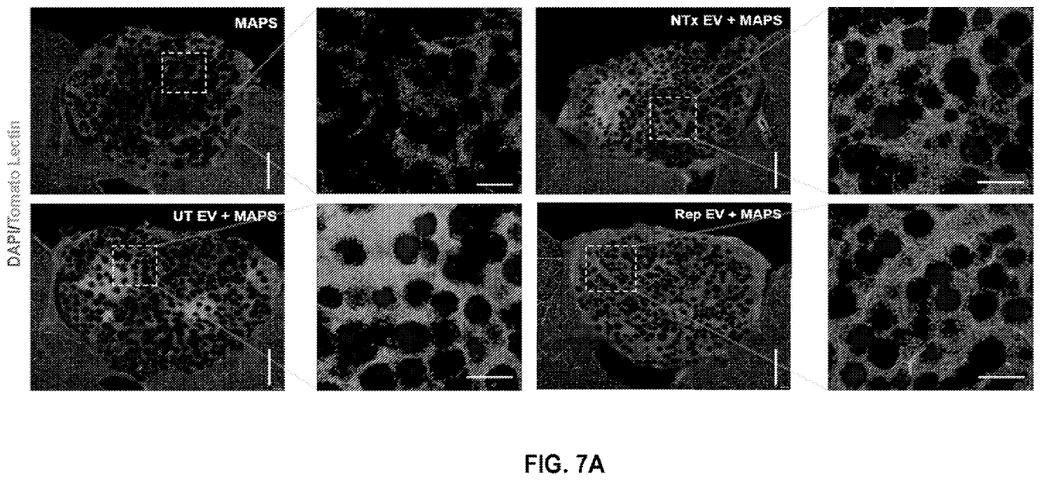

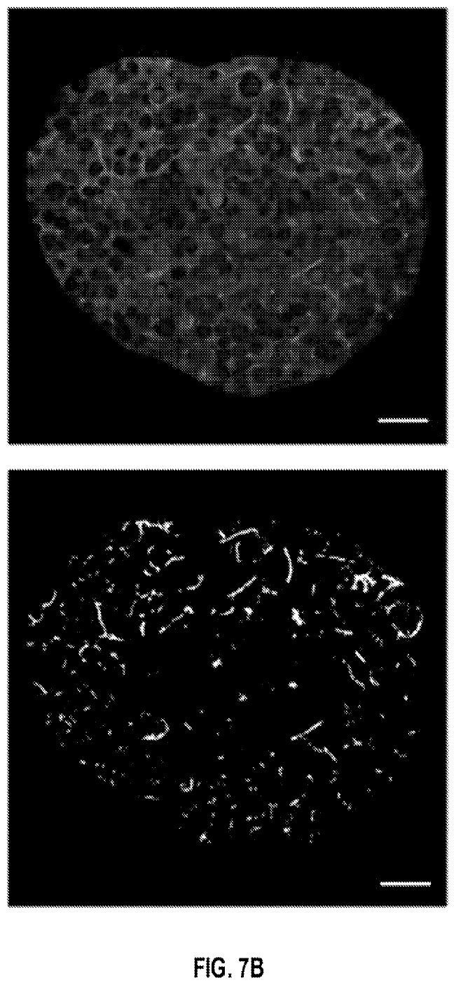

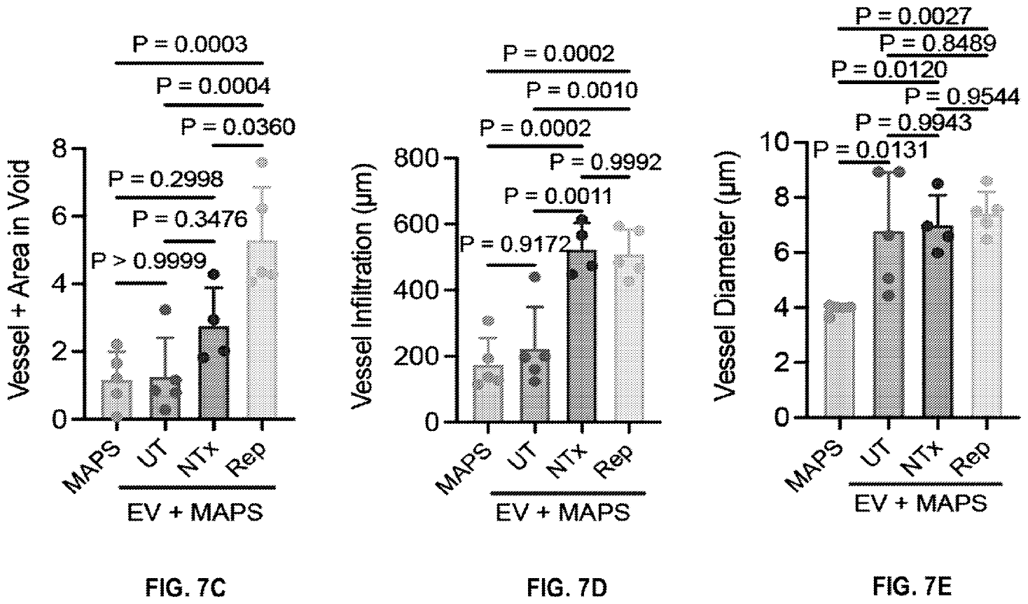

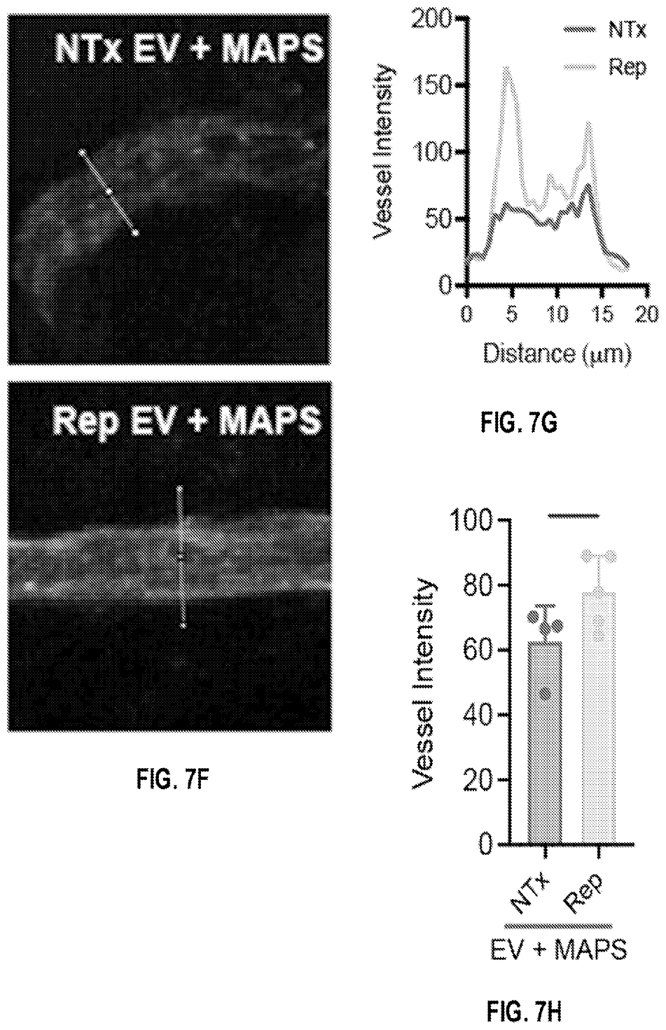

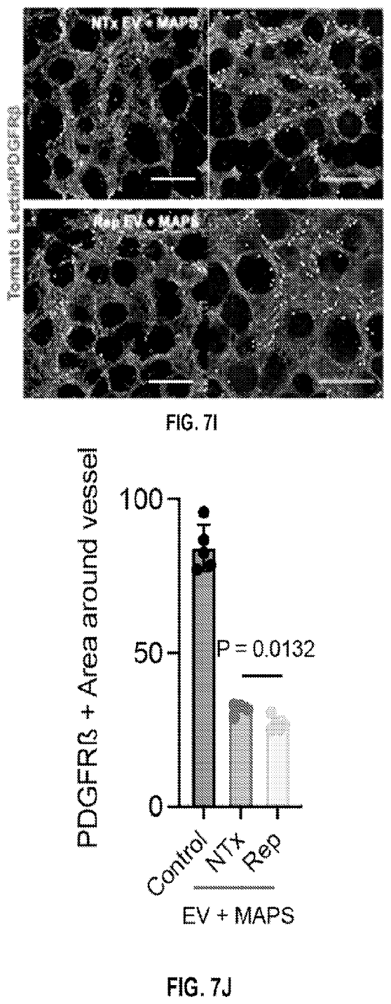

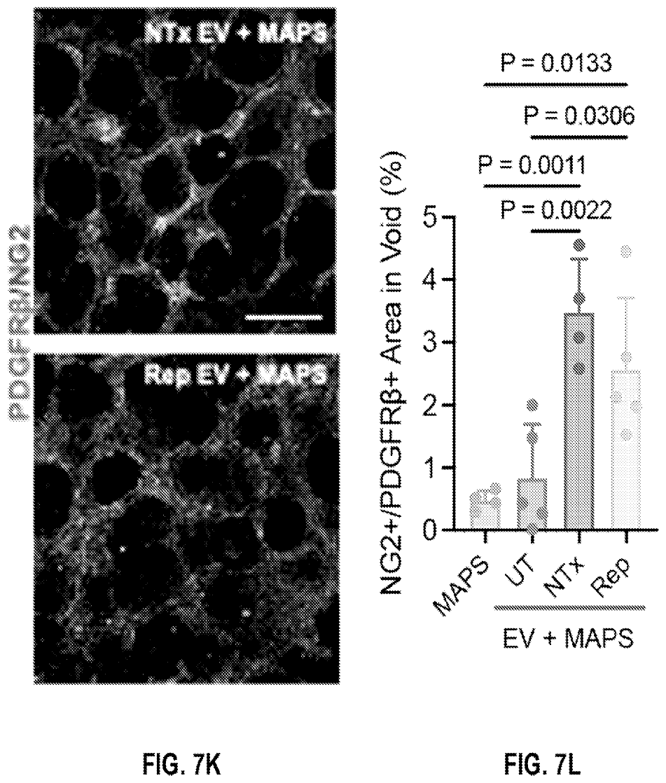

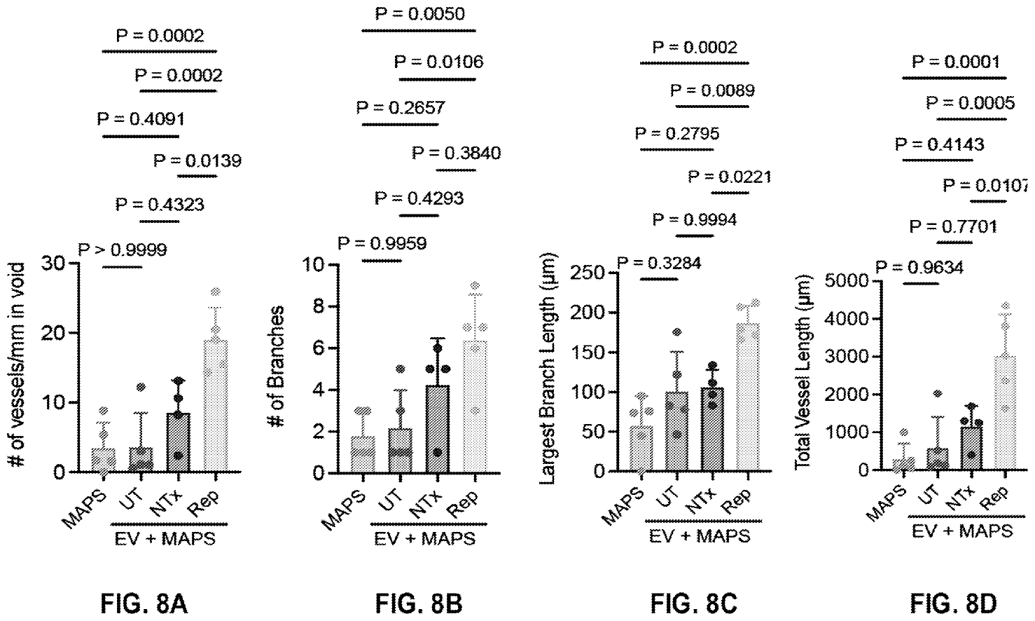

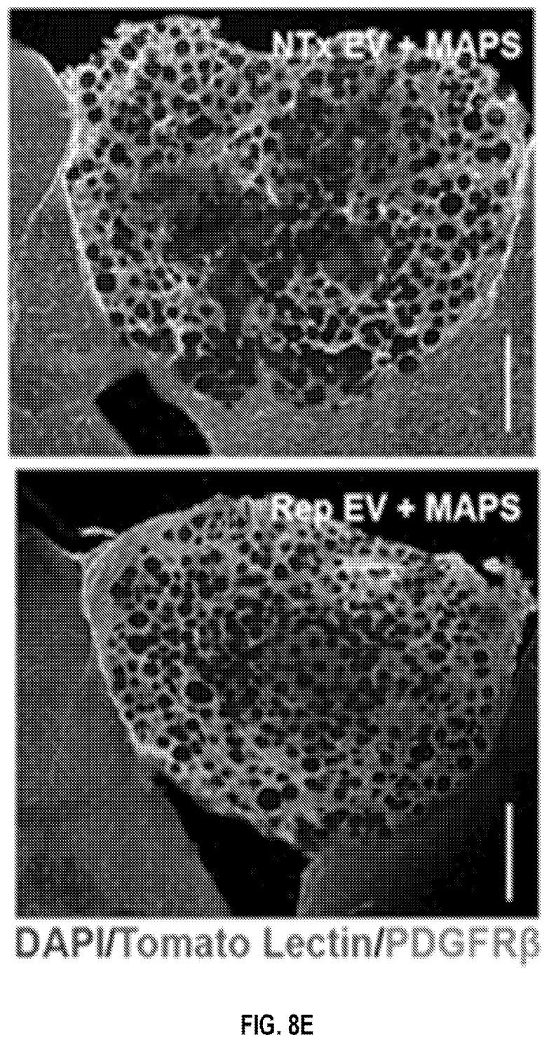

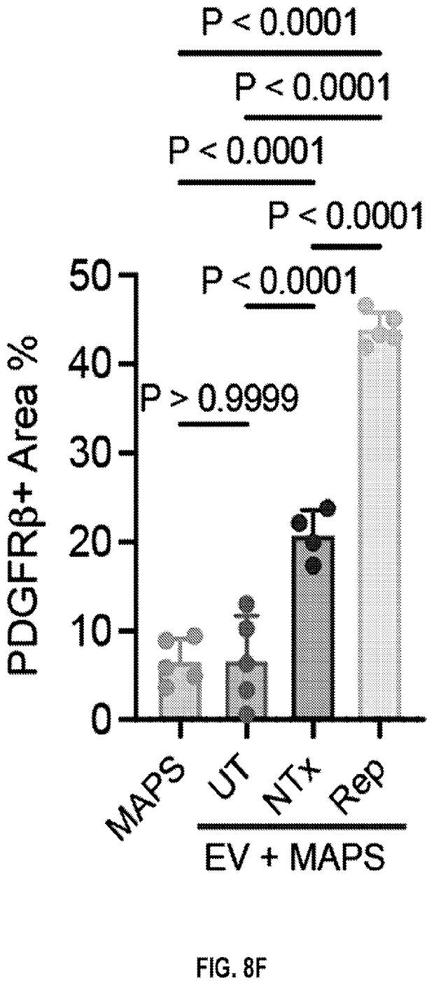

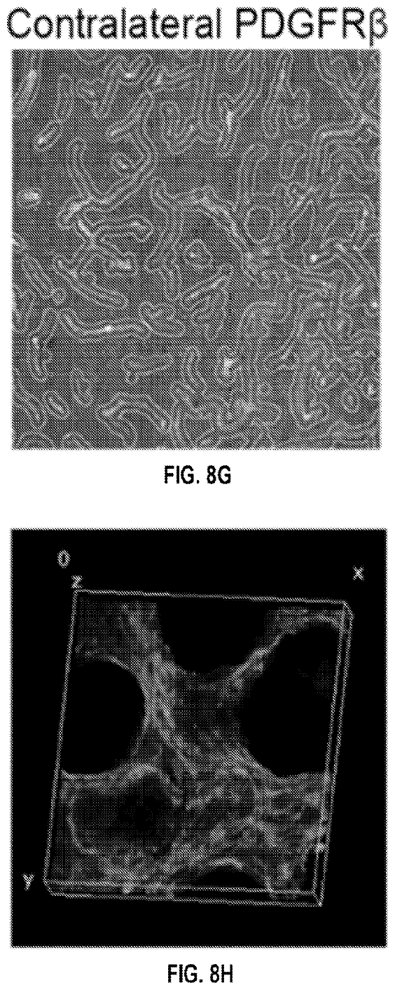

FIG. 7A-7E shows that Rep-EV+MAPs improves angiogenesis and induces pericyte infiltration 21 days after stroke. As shown in FIG. 7A, gross observation revealed that NTx- and Rep-EV+MAPS groups had obvious vessels in the middle of the infarct, while UT-EV+MAPS and MAPS only groups did not. As expected, the vessels grew surrounding the microgels that form MAPS rather than penetrating through them (FIG. 7A, insets), indicating that the void structure in MAPS can be remodeled in situ to support vessel growth. To quantify the density of the vasculature in the voids the positive area of tomato lectin stain was measured as well as the number of vessels per mm in a binarized image (FIG. 7B). To assess the vessel infiltration distance, the distance from the edge of the stroke to the furthest vessel found in the section was measured. Finally, to assess vessel morphology vessel branching and vessel thickness was assessed. Overall, new blood vessel formation within stroke infarct in NTx- and Rep-EV+MAPS groups was significantly improved compared to UT-EVs+MAPS and MAPS only groups (FIGS. 7C-7E and FIGS. 8A-8D).

Both NTx- and Rep-EV groups had similar branch counts and blood vessel infiltration, but the total blood vessel area within the infarct and maximum branch length were higher in Rep-EV group, indicating increased angiogenic cargo in Rep-EVs compared to NTx-EVs. In addition, the new vessels in Rep-EV group had higher tomato lectin fluorescent intensity than those in NTx-EV group, indicating Rep-EV-MAPS promoted the regeneration of blood vessels with a more mature glycocalyx (FIGS. 7F-7H). Notably there are perfused vessels spanning the center of the infarct in both NTx- and Rep-EV conditions, a tissue that never recovers from stroke. This indicates that reactive astrocytic EVs impart a significant reparative response after stroke, after only 16 days EV+MAPS injection.

PDGFRβ staining, as a marker of pericytes, was used as to further study the integrity of new blood vessels in the collected samples. A robust PDGFRβ+ cell population was found in the infarct region (FIGS. 8E-8F). To determine the % PDGFRβ+ area in mature vessels the % PDGFRβ+ area was measured in the contralateral side using a vessel ROI such that only PDGFRβ+ area in vessels was captured. It was found that 84% PDGFRβ+ area in the vessel ROI for the contralateral vessels (FIG. 7I, FIG. 7J and FIG. 8G), while for Rep-EV and NTx-EV+MAPS treatments only 25-30% PDGFRβ+ area was found in the vessel ROI, indicating that the new blood vessels in the infarct are less mature than those in the contralateral side. Since the PDGFRβ stain was not completely specific to pericytes, pericytes were further defined as cells co-expressing PDGFRβ and NG2 as described in REF 26. Co-expressing cells (PDGFRβ+ and NG2+) were found to colocalize with vessels and to have a statistically higher positive area in the Rep-EV and NTx-EV+MAPS groups (FIG. 7K, FIG. 7L and FIG. 8H, FIG. 8I). These results demonstrate that 16 days-post AD-EV+MAP injection, vessels are beginning to be remodeled to become mature.

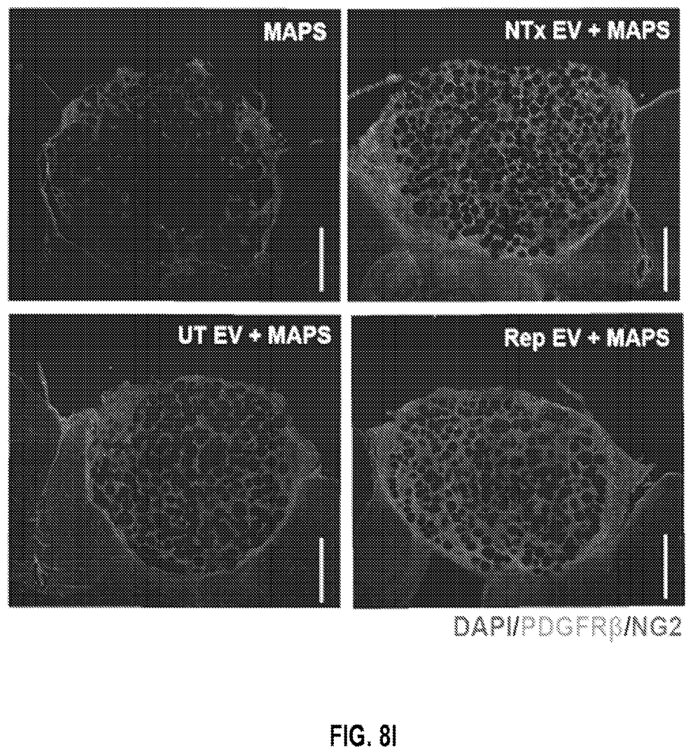

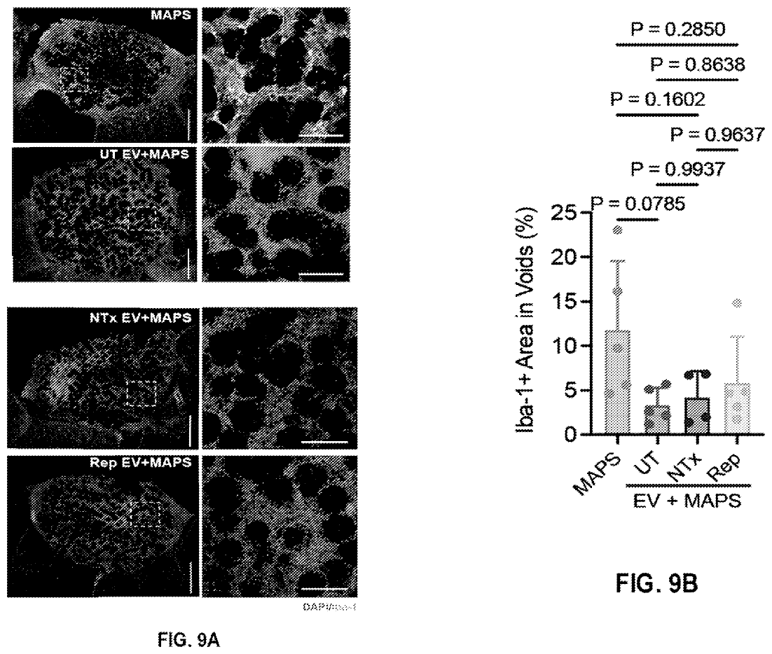

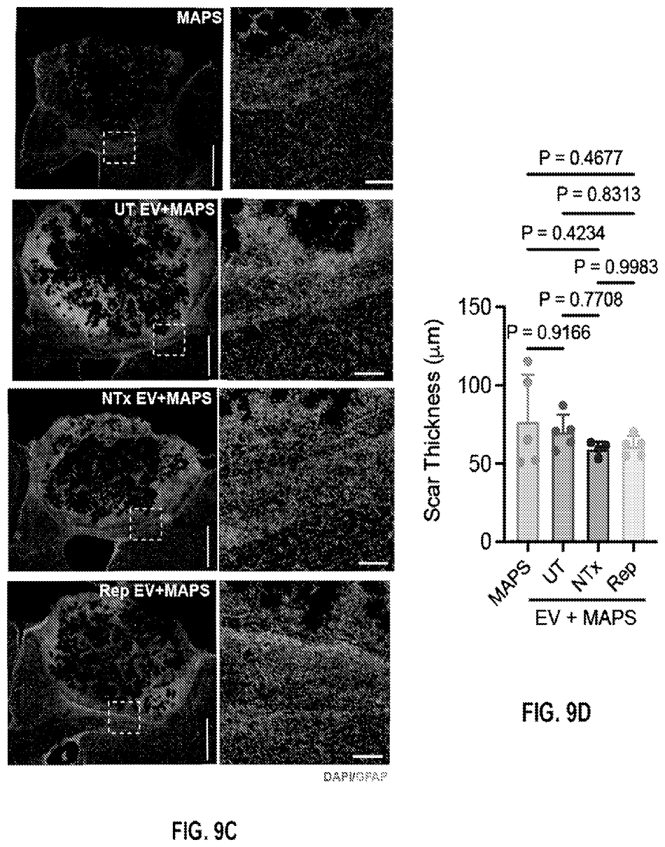

The overall PDGFRβ+ area in the void showed a significant increase for Rep-EV and NTx-EV+MAPS condition compared to MAPS and UT-EV+MAPS condition, with 45% and 20% PDGFRβ+ area, respectively (FIG. 8F). These results demonstrate that a PDGFRβ+ cell population contributes significantly to the remodeled void space that leads to regenerative processes after stroke in the Rep-EV and NTx-EV+MAPS groups, which could possibly be oligodentrocyte progenitor cells and fibroblast. The majority of infiltrating cells in MAPS only group were Iba-1+ microglia/macrophages (FIG. 9A, FIG. 9B), whereas EV+MAPS groups had limited amount of Iba-1+ cells, suggesting AD-EVs promoted transition from immune responses to regenerative process. The scar thickness was unchanged between EV+MAPS groups and MAPS only group, but all these groups had reduced scar compared to sham or EV only group (FIG. 4E, FIG. 4F, FIG. 9C-9D).

### Example 7: ADEV-MAPs Derived from Neurotoxic Astrocytes Improve Axonal Sprouting after Stroke

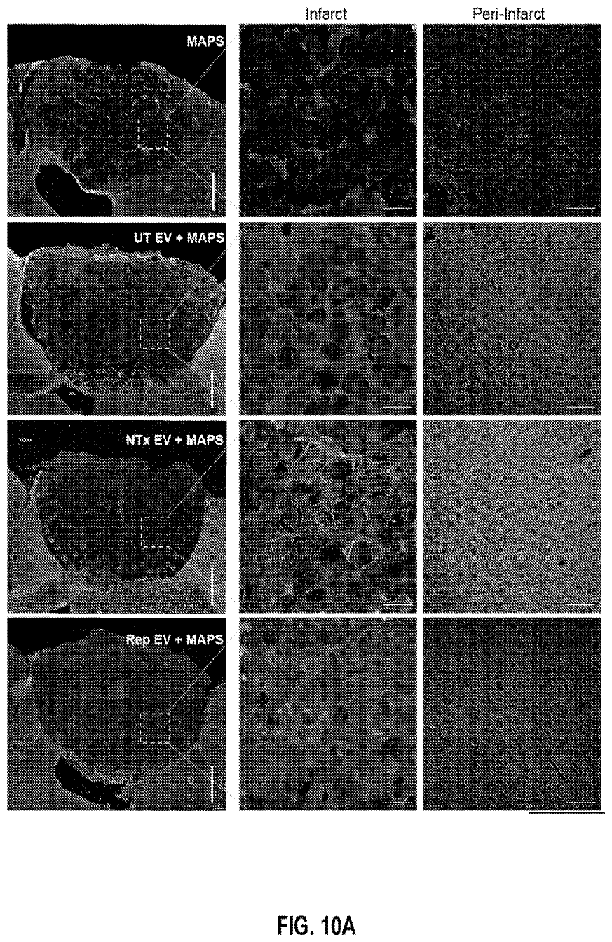

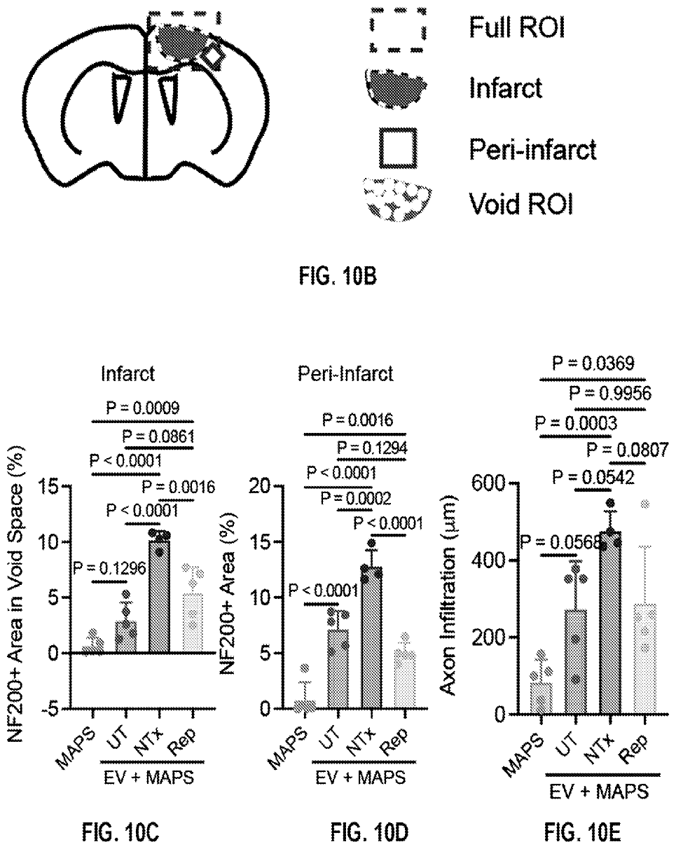

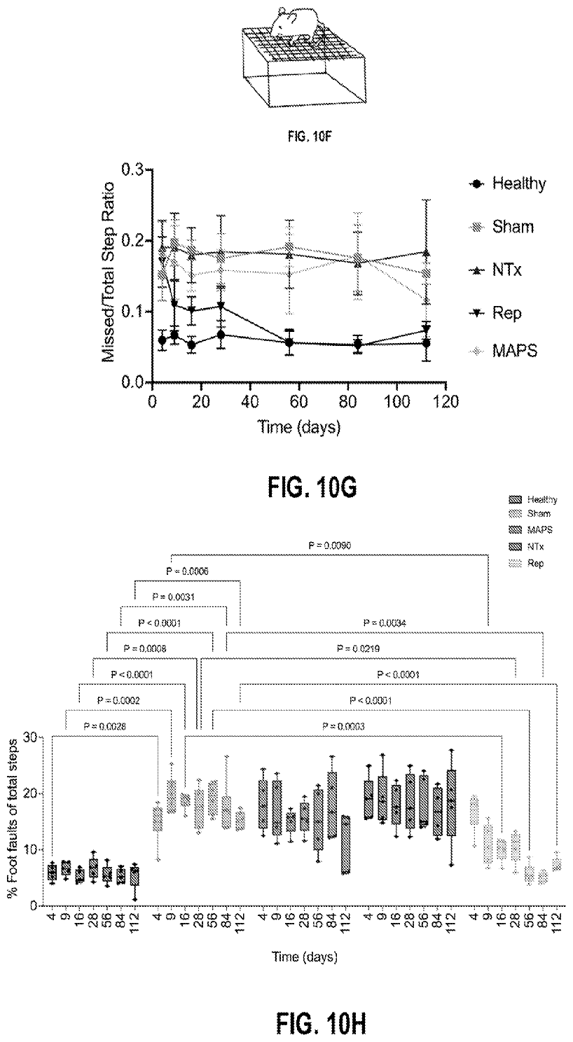

The treated and collected tissues from Examples 5 and 6 were further analyzed for signs of axonal sprouting and neurogenesis. Neurotoxic astrocytes have been found to kill neurons by secreting APOE and APOJ saturated lipids (see e.g., Guttenplan, K. A. et al. Nature 599, 102-107 (2021), incorporated herein by reference in its entirety) and post stroke axonal sprouting is necessary for behavioral improvement after stroke (see e.g., Benowitz, L. I. & Carmichael, S. T. Neurobiology of disease 37, 259-266 (2010), incorporated herein by reference in its entirety). Therefore, tissues were stained for NF200 (neurofilament) as a measure of axonal sprouting. Both NTx- and Rep-EV groups resulted in increased neurofilament area (as measured by NF200 staining) in the infarct and peri-infarct compared to MAPS only (FIG. 10A-10E). However, NTx-EV+MAPS group had the highest NF200 neurofilament area in infarct and peri-infarct as well as NF200 infiltration distance (FIG. 10C-10E), despite being derived from neurotoxic astrocytes suggesting that harmful saturated lipids are not presented in NTx-EVs. This data indicates that NTx-EV+MAPS can improve axon regrowth at and Rep-EV+MAPS can promote functional recovery.

### Example 8: Rep-EV Promote Functional Recovery after Stroke

To evaluate the functional recovery after stroke as results of implanting ADEV-MAPS scaffolds, grid walking behavioral studies were performed on mice treated with each experimental condition (FIG. 10F). To quantify effects on behavior, the ratio of missed and total steps was determined (FIG. 10G, FIG. 10H). First a baseline comparison between healthy and stroked mice was obtained where it was found the stroked mice had around 2.5-fold higher missed/total step ratio compared to healthy mice throughout the 4-month period. The three EV+MAPS groups (UT, Rep, NTx) had similar percentage of missed steps at Day 4 compared to stroked mice, but Rep-EV+MAPS group significantly decreased the percentage of missed steps at Day 16 compared to sham, while MAPS and NTx-EV+MAPS did not show any improvement. The mice in Rep-EV+MAP group continued to recover and have a similar behavior output as healthy mice after two months (FIG. 10G, FIG. 10H).

These results collectively showed that reactive astrocytes that express both inflammatory and reactive genes, have EVs with pro-reparative cargos capable of promoting tissue repair, while reactive astrocytes that express less inflammatory genes have EVs that promote functional recovery after stroke.

### Example 9: Proteomics Profiling of Proteins Components within UT, NTx, and Rep-EVs

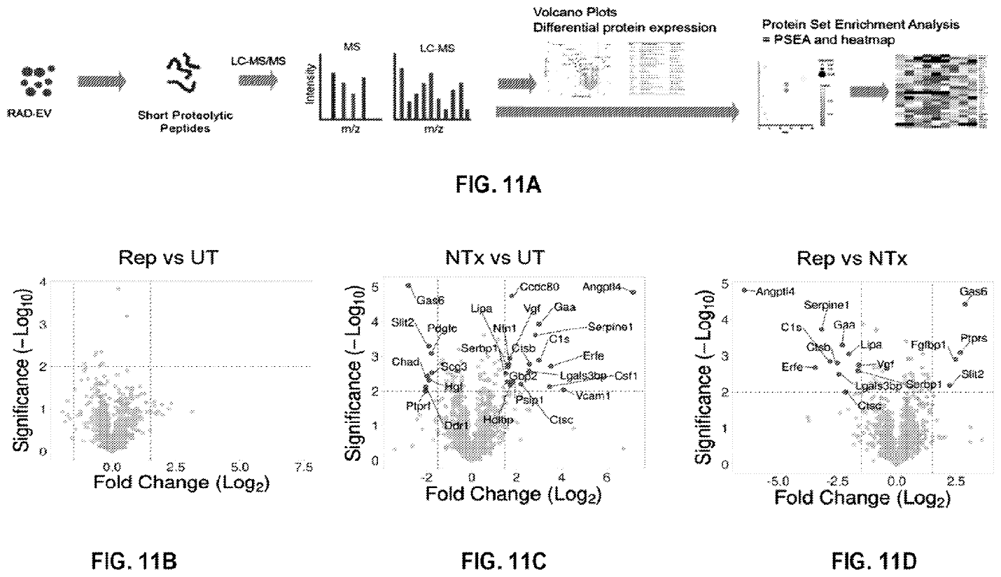

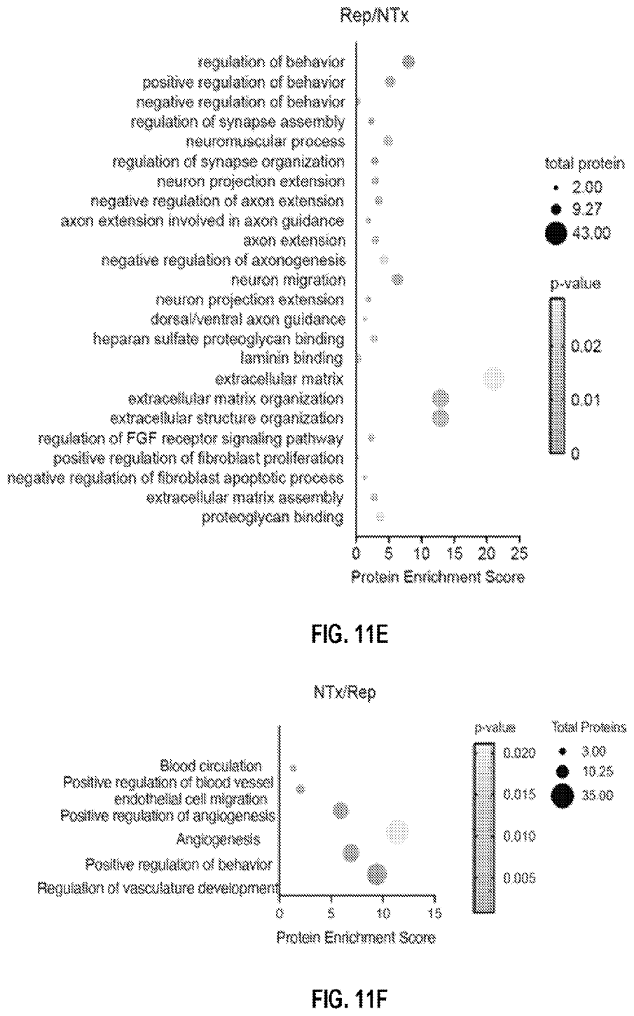

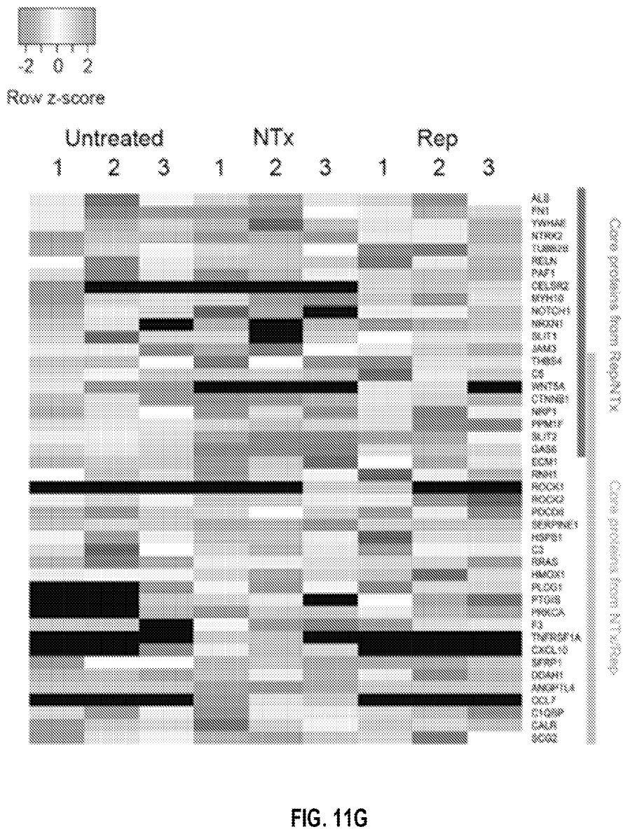

Due to the differential tissue repair results from EV+MAPS scaffolds derived from UT, NTx, and Rep astrocytes, protein cargo identity and levels across the three sets were analyzed to mechanistically explain differences differences observed. Specifically, proteomic analysis was performed to identify the key protein components and pathways leading to regeneration in each group. First, the astrocytic EVs were lysed after collection as described in Example 1 and analyzed with nanoscale liquid chromatography coupled to tandem mass spectrometry (nanoLC-MS/MS) and label-free quantification (FIG. 11A).

Specifically, The ADEV pellets obtained after isolation were resuspend in 8 M urea, 50 mM ammonium bicarbonate in H₂O with protease inhibitors. These solutions were mixed and sonicated for 5 cycles (30 sec on, 30 sec off). The total protein quantity within these samples were determined by micro BCA assay. The samples were then spiked with 6-8 M urea/50 mM Tris-HCl (pH=8) and reduced with 5 mM dithiolthreitol for 30 mins at 37° C. and alkylated with 15 mM iodoacetamide for 30 mins at room temperature in the dark. After 3X urea and Tris washes, proteins were digested using trypsin for overnight at 37° C. and eluted using 0.001% Zwittergent 3-16 and 1% HCOOH. Samples were dissolved in 200 μL 0.001% Zwittergent 3-16 prior to nanoLC-MS/MS.

Quantitative LC-MS/MS was performed on 2 μl of each sample, using an EASY nanoLC-1200 coupled to a Thermo Scientific Orbitrap Exploris-480 via an EASY-Spray source. In brief, the sample was first trapped on a Thermo Scientific Acclaim PepMap 100 C₁₈ nanoLC 20 mm×75 μm trapping column (5 μl/min at 99.9/0.1 v/v water/acetonitrile), after which the analytical separation was performed using an EASY-Spray C18 nanoLC 75 μm×250 mm column with a 150-min linear gradient of 5-95% acetonitrile with 0.1% formic acid at a flow rate of 300 nl/min with a column temperature of 55° C. Data collection on the Orbitrap Exploris-480 mass spectrometer was performed in a data-dependent acquisition (DDA) mode of acquisition with r=120,000 (at m/z 200) full MS scan from m/z 375 to 1,600 with a target AGC value of 2×10⁵ ions. MS/MS scans were acquired at rapid scan rate in the linear ion trap with an AGC target of 5×10³ ions and a max injection time of 100 ms. The total cycle time for MS and MS/MS scans was 1.5 s. A 20 s dynamic exclusion was employed to increase depth of coverage.

Proteome Discoverer 2.5 (Thermo Scientific) was used for database searching of the experimental nanoLC-MS/MS data against protein sequences for Rattus norvegicus (SwissProt, reviewed, TaxID=10116). Mascot Distiller and Mascot Server were used to produce fragment ion spectra and to perform the database searches using full trypsin enzyme rules with 5 ppm precursor and 0.02 Da product ion match tolerances. Database search parameters included static modification on cysteine (carbamidomethyl) and dynamic modifications on N-terminal acetylation, methionine loss, and methionine loss plus acetylation. Peptide Validator and Protein FDR Validator nodes in Proteome Discoverer were used to annotate the data at a maximum 1% protein false discovery rate.

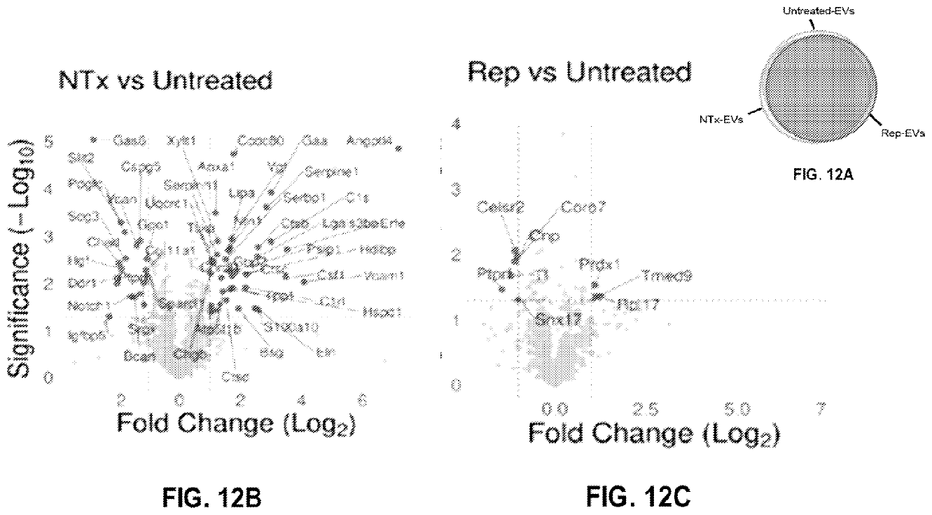

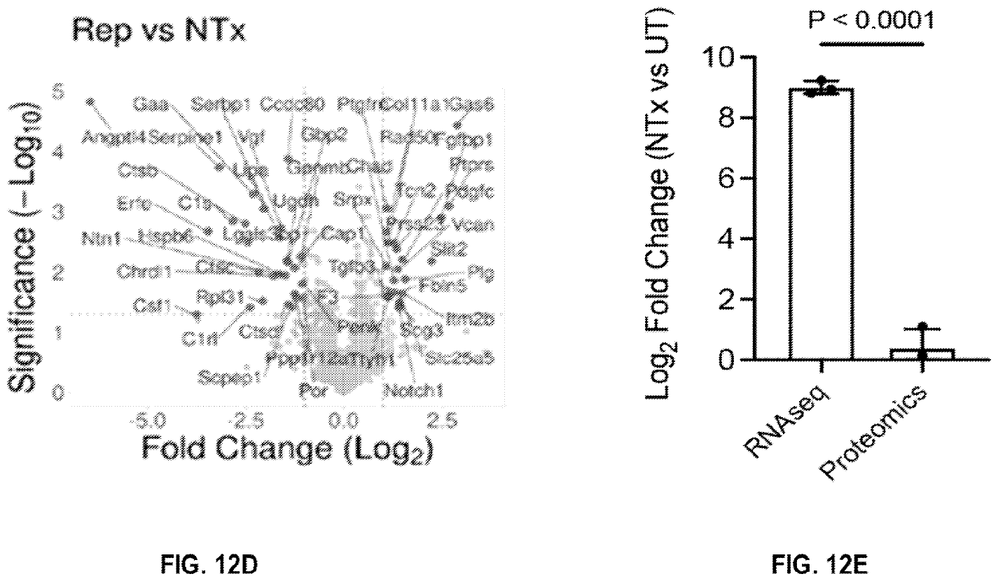

In total, 1073 proteins were detected from all samples and 94% of these proteins are congruent between Rep, NTx, and UT-EV groups (FIG. 12A). The label-free quantification showed the protein abundances in NTx-EVs were drastically different compared to UT EVs, whereas the protein profile in Rep-EVs was similar to UT EVs (FIGS. 11B-11D and FIGS. 12B-12D). These trends are similar to the gene expression data (FIG. 2A), indicating the astrocytic EVs contain protein cargos that correlate with their parent cells. Proteins in Rep-EVs were compared to NTx-EVs using significance ratio of 2.0 and fold change of 1.5 and as shown in FIGS. 11B-11D and Table 3 there were 4 up-regulated proteins, including Gas6 involved in reoxygenation (see e.g., Tong, L.-s. et al. Journal of Cerebral Blood Flow & Metabolism 37, 1971-1981 (2017), PTPRS relating to synapse formation (see e.g., Takahashi, H. & Craig, A. M. Trends in neurosciences 36, 522-534 (2013), FGFBP1 involved in regeneration (see e.g., Taetzsch, T. et al., Biochimica et Biophysica Acta (BBA)-Molecular Basis of Disease 1864, 2983-2991 (2018)), and SLIT2 involved in BBB repair (see e.g., Sherchan, P. et al. Scientific reports 7, 1-11 (2017), all of which are incorporated herein by reference in their entirety. In addition, 11 down-regulated proteins were observed including neuronal growth factor VGF (FIGS. 11B-11D, Table 3). These proteins are possible key players contributing to the angiogenesis in Rep-EV+MAPS (FIGS. 7A-7L) and axonogenesis in NTx-EV+MAPS (FIGS. 10A-10H) treated animals.

TABLE 3

| Relative Expression | | |
|---|---|---|
| (Upregulated or | | |
| Downregulated) | Protein | Role in CNS |
| Upregulated | GAS6 | Reoxygenation |
| Upregulated | PTPRS | Promotes synapse |
| | | formation |
| Upregulated | FGFBP1 | Regeneration |
| Upregulated | SLIT2 | BBB repair |
| Downregulated | VGF | Regeneration |
| Downregulated | SERBP1 | Matrix remodeling |
| Downregulated | LIPA | Lipid metabolism |
| Downregulated | CTSC | Matrix remodeling |
| Downregulated | GAA | Glycosidation |
| Downregulated | LGALS3BP | NPC migration |
| Downregulated | CTSB | Matrix remodeling |
| Downregulated | C1S | Inflammation |
| Downregulated | SERPINE1 | Matrix remodeling |
| Downregulated | ERFE | RBC Iron Availability |
| Downregulated | ANGPTL4 | BBB repair |

Table 4 provides presence of reactive and/or inflammatory genes/proteins between astrocyte RNA sequencing and EV proteomics data. Interestingly, many inflammatory genes found in RNA sequencing data were not presented as proteins in EVs, especially for NTx astrocytes.

TABLE 4

| Gene/Protein | Astrocytes | EVs |
|---|---|---|
| Lcn2 | Yes | No |
| Steap4 | Yes | No |
| S1pr3 | Yes | No |
| Osmr | Yes | No |
| Aspg | Yes | No |
| Serping1 | Yes | No |
| Ggtg1 | Yes | No |
| Fkbp5 | Yes | No |
| Psmb8 | Yes | No |
| Cxcl1 | Yes | No |
| Ccl2 | Yes | No |
| IL6 | Yes | No |
| Clcf1 | Yes | No |
| Lif | Yes | No |
| IL13ra1 | Yes | No |
| Tapbp | Yes | No |
| B2m | Yes | No |
| C1r | Yes | No |
| C4b | Yes | No |
| Timp1 | Yes | Yes |
| Hspb1 | Yes | Yes |
| Cxcl10 | Yes | Yes |
| Cd44 | Yes | Yes |
| Cp | Yes | Yes |
| Vim | Yes | Yes |
| Gfap | Yes | Yes |
| Gbp2 | Yes | Yes |
| Fbln5 | Yes | Yes |
| Srgn | Yes | Yes |
| C3 | Yes | Yes |
| C1s | Yes | Yes |
| Serpina3n | No | No |
| ligp1 | No | No |
| Ugt1a1 | No | No |
| Amigo2 | No | No |

NTx astrocytes had a 9.005 log 2 fold change of C3, the most characteristic and highly up-regulated marker for NTx astrocytes, at the cell level compared to UT astrocytes, but only 0.396 log 2 fold change in EV level (FIG. 12E), suggesting the reduced toxicity in NTx-EVs and explaining why axon regeneration was induced by NTx-EV+MAPS.

Because most of the proteins are the same among UT, NTx, and Rep-EVs, the abundance ratios for enrichment analysis was analyzed to obtain significantly up-regulated pathways in a group. Therefore, protein set enrichment analysis (PSEA)-Quant of abundance ratios between Rep/NTx and NTx/Rep was used to find gene ontology pathways of reparative processes (FIG. 11E, FIG. 11F). Surprisingly, Rep-EVs have 14 neuronal growth pathways significantly up-regulated, including axon extension involved in axon guidance (GO:0048846), regulation of synapse assembly (GO:0051963), dorsal/ventral axon guidance (GO:0033563), neuron projection extension (GO:1990138), neuron migration (GO:0001764), and axon extension (GO:0048675). Furthermore, there were 7 ECM protein related pathways up-regulated in Rep-EVs, including extracellular matrix (GO:0031012), proteoglycan binding (GO:0043394), extracellular matrix organization (GO:0030198), extracellular structure organization (GO:0043062), and heparan sulfate proteoglycan binding (GO:0043395). Rep-EV+MAPS group was the only treatment group that had a significant behavioral improvement, suggesting that these reparative Gene Ontology pathways play a significant role in recovery after stroke.

NTx-EVs also up-regulate pathways compared to Rep-EVs, which were mostly angiogenesis related, including angiogenesis (GO:0001525), positive regulation of behavior (GO:0048520), and regulation of vasculature development (GO:1901342), which may contribute to the improved PDGFRβ+ cells surrounding vessels in NTx-EV+MAPS group.

Core proteins from PSEA-Quant were plotted in a heatmap to show the difference of expression levels among the three groups (FIG. 11G). Rep-EVs group had higher expression of most of the core proteins from Rep/NTx analysis including some regenerative proteins, such as FN1, THBS4, and RELN, indicating these proteins probably contributed to the tissue regeneration and behavioral recovery in Rep-EVs group. Apart from these core proteins, NTx-EVs had more expression of axon permissive factors NTN1 (see e.g., Huang, Q. et al. ACS Applied Materials & Interfaces 13, 112-122 (2021) which is incorporated herein by reference in its entirety) and VGF and less expression of axon inhibitory factors, such as BCAN, VCAN, SLIT2 (see e.g., Anderson, M. A. et al. Nature 532, 195-200 (2016), which is incorporated herein by reference in its entirety), which possibly leads to improved axon infiltration in NTx-EVs group (FIG. 10A-10H).

### Example 10: Preparation of TDEV-HMPs and TDEV-MAP Hydrogels EVs

This example describes methods for deriving extracellular vesicles (EVs) from T-cells, immobilization to HMPs and preparation of MAP hydrogel scaffolds including these activated T-cell derived EVs.

CD4+ T helper cells were prepared from 8-week old mouse spleens using EasySep mouse CD4+ T cell isolation kit (Stemcell). The cells were cultured in RPMI1640 supplemented with 10% FBS, NEAA, antibiotics, and 55 μM β-mercaptoethanol with the inducing cytokines (0.5 μg/mL anti-CD28 and 1 μg/mL anti-IL-4 antibodies, 5 ng/ml IL-2, and 10 ng/ml IL-12 for T helper 1 (Th1) activation, 0.5 μg/mL anti-CD28 and 1 μg/mL anti-IFN-γ antibodies, 5 ng/mL IL-2, and 10 ng/ml IL-4 for T helper 2 (Th2) activation, and 0.5 μg/mL anti-CD28, 1 μg/mL anti-IFN-γ, and 1 μg/mL anti-IL-4 antibodies, and 2 ng/ml TGF-β1 for regulatory T cells (Treg). After 96 hours of culture, the culture media were collected and centrifuged at 2000 g for 30 mins to remove cells and debris. The supernatant was transferred into a new tube and mixed with half volume of the total exosome isolation kit (Thermo Scientific) for overnight incubation at 4° C. The solutions were then centrifuged at 10,000 g for 60 mins at 4° C. and the resulted Th cell EV pellets were ready to be resuspended in PBS for downstream process.

Th cell-EVs were conjugated to hyaluronic acid based microporous annealed particle hydrogels (MAPS) using the same methods described in Example 4 above). Th cell-EV+MAPS were transplanted into stroke cavity in a mouse stroke model and the brain repair was evaluated as described in Examples 5-8 above. The number of Th cell-EV transplanted into mouse were kept same across the groups, which is adjusted based on the EV concentration data measured by nanoparticle tracking analysis.

### Example 11: The Cell-EV+MAPS Effect on Brain Repair

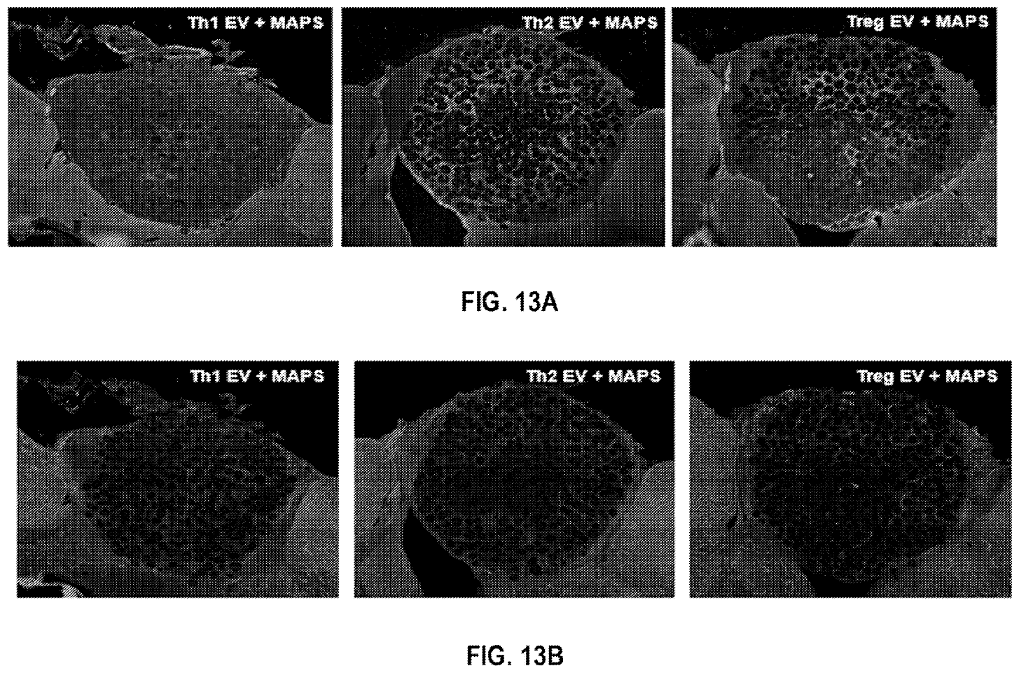

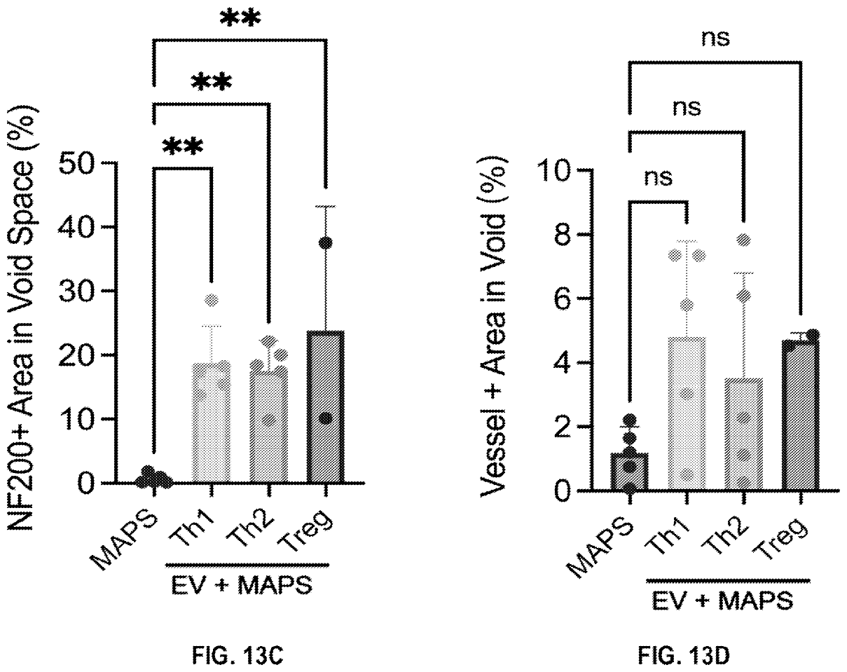

FIGS. 13A-13D shows the brain repair outcomes induced by Th cell-EV+MAPS at 21 days after stroke. Specifically, FIG. 13A and FIG. 13C shows that axon infiltration (measured by NF200 staining) into the stroke cavity was significantly increased with the addition of all Th cell-EV groups as compared to MAPS only. Further, in FIG. 13B and FIG. 13D, it is shown that the new blood vessel formation was enhanced about two-fold across the groups, without any significant differences. Compared to astrocyte derived EV groups, Th cell-EV groups show better axon infiltration that takes up 20% of the void space in stroke cavity (FIG. 13C), but the angiogenesis outcomes were not found to be statistically significant between the four groups (FIG. 13D).

1. An extracellular vesicle hydrogel microparticle (EV-HMP), the microparticle comprising: (i) a hydrogel microparticle (HMP) and (ii) an extracellular vesicle (EV) derived or collected from a cell population, wherein the extracellular vesicle is immobilized to the HMP.

2. The EV-HMP of claim 1, wherein the extracellular vesicle is labeled with an azido sugar and the hydrogel microparticle is functionalized with a strained alkyne.

3. The EV-HMP of claim 2, wherein the extracellular vesicle (EV) is immobilized to the hydrogel microparticle (HMP) via a triazole linkage between the azido sugar and the strained alkyne.

4. A method of making an extracellular vesicle hydrogel microparticle (EV-HMP), the method comprising:
(a) isolating an extracellular vesicle labeled with an azido sugar (azido-EV) from a cell population; and
(b) contacting the azido-EV with a hydrogel microparticle (HMP) functionalized with a strained alkyne so that the azido sugar of the azido-EV forms a stable triazole linkage with strained alkyne of the HMP to make the extracellular vesicle hydrogel microparticle (EV-HMP).

5. The method of claim 4, wherein step (b) comprises incubating the azido-EV with the hydrogel microparticle at an elevated temperature for at least 10 minutes, at least 15 minutes, at least 20 minutes, at least 25 minutes, or at least 30 minutes.

6. The method of claim 5, wherein the elevated temperature is about 30° C. to about 40° C.

7. The method of any one of claims 4 to 6, further comprising culturing the cell population in the presence of the azido sugar before isolating the azido-EV from the population.

8. The EV-HMP of any one of claims 2 to 3 or the method of any one of claims 4 to 7, wherein the azido sugar comprises N-azidoacetylmannosamine-tetraacylated (Ac4MannAz), 9-azido sialic acid, 6-Azide-Trehalose (6-TreAz), 8-Azido-3,8-dideoxy-D-manno-octulosonic acid (Kdo Azide), 9-azido-9-deoxy-N-acetylneuraminic c acid (9AzNeu5Ac), N-azidoacetylglucosamine-tetraacylated (Ac4GlcNAz), N-azidoacetylgalactosamine-tetraacylated (Ac4GalNAz), 6-azido-6-deoxy-N-acetyl-glucosamine triacylated (Ac3-6AzGlcNAc), or any combination thereof.

9. The EV-HMP of any one of claims 2 to 3 and 8 or the method of any one of claims 4 to 8, wherein the strained alkyne comprises dibenzocyclooctyne (DBCO), bicyclo[6.1.0]non-4-yn-9-ylmethanol (BCN), azadibenzocyclooctyne (ADIBO), cyclooctyne (OCT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or any combination thereof.

10. The EV-HMP of any one of claims 1 to 3 and 8 to 9 or the method of any one of claims 4 to 9, wherein the hydrogel microparticle comprises hyaluronic acid, Poly (ethylene glycol), gelatin, alginate, collagen, Methyl cellulose, or any combination thereof.

11. The EV-HMP of any one of claims 1 to 3 and 8 to 10 or the method of any one of claims 4 to 10, wherein the cell population comprises astrocytes or T-cells.

12. The EV-HMP of any one of claims 1 to 3 and 8 to 11 or the method of any one of claims 4 to 11, wherein the cell population is cultured in a medium comprising at least one cytokine or cytokine antibody.

13. The EV-HMP or method of claim 12, wherein the cell population is cultured in a medium comprising at least one cytokine or cytokine antibody for at least 10, at least 15, at least 20, or at least 24 hours.

14. The EV-HMP or method of claim 12 or 13, wherein the medium comprising at least one cytokine or cytokine antibody comprises an anti-CD28 antibody, an anti-IL4 antibody, IL-2, IL-12, an anti-IFNγ antibody, IL-4, TGFβ1, IL-1α, TNF, C1q, or any combination thereof.

15. The EV-HMP or method of claim 14, wherein the cell population comprises astrocytes and the medium comprising the at least one cytokine or cytokine antibody comprises IL-4, IL-1α, TNF, C1q, or any combination thereof.

16. The EV-HMP or method of claim 15, wherein the medium comprising the at least one cytokine or cytokine antibody comprises (a) IL-1α, TNF, and/or C1q; or (b) IL-4 and/or C1q.

17. The EV-HMP or method of claim 14, wherein the cell population comprises T-cells and the medium comprising the at least one cytokine or cytokine antibody comprises anti-CD28 antibody, anti-IL4 antibody, IL-2, IL-12, anti-IFNγ antibody, IL-4, TGFβ1, or any combination thereof.

18. The EV-HMP or method of claim 17, wherein the medium comprising the at least one cytokine or cytokine antibody comprises (a) anti-CD28 antibody, anti-IL4 antibody, IL-2, and/or IL-12; (b) anti-CD28 antibody, anti-IFNγ antibody, IL-2, and/or IL-4; or (c) anti-CD28 antibody, anti-IFNγ antibody, anti-IL4 antibody, and/or TGFβ1.

19. The EV-HMP or method of any one of claims 14 to 18, wherein the medium comprising the at least one cytokine or cytokine antibody comprises from about 0.05 to about 3 μg/mL of the anti-CD28 antibody, about 0.5 to about 5 μg/mL of the anti-IL4 antibody, about 1 to about 10 ng/ml of the IL-2, about 5 to about 20 ng/ml of the IL-12, about 0.5 to about 5 μg/mL of the anti-IFNγ antibody, about 5 to about 20 ng/mL of the IL-4, about 0.5 to about 5 ng/ml of the TGFβ1, about 1 to about 5 ng/ml of the IL-1α, about 10 to about 50 ng/ml of the TNF, about 100 to about 800 ng/ml of the C1q, or any combination thereof.

20. The EV-HMP or method of claim 19, wherein the medium comprising the at least one cytokine or cytokine antibody about 0.5 μg/mL of the anti-CD28 antibody, about 1 μg/mL of the anti-IL4 antibody, about 5 ng/ml of the IL-2, about 10 ng/ml of the IL-12, about 1 μg/mL of the anti-IFNγ antibody, about 10 ng/mL of the IL-4, about 2 ng/ml of the TGFβ1, about 3 ng/ml of the IL-1α, about 30 ng/ml of the TNF, about 400 ng/ml of the C1q, or any combination thereof.

21. The EV-HMP or method of any one of claims 12 to 20, wherein the medium comprising the at least one cytokine or cytokine antibody further comprises an extracellular vesicle collecting media (EV collecting media).

22. The EV-HMP or method of claim 21, wherein the EV collecting media comprises Dulbecco's Modified Eagle Medium (DMEM), FBS, hydrocortisone, an antibacterial agent, glutamine, insulin, NaPyruvate, N-acetyl-L-cysteine or any combination thereof.

23. An EV-HMP prepared by the method of any one of claims 4 to 22.

24. An EV-HMP suspension comprising two or more EV-HMPs of any one of claims 1 to 3 and 8 to 23.

25. An extracellular vesicle microporous annealed particle hydrogel (EV-MAP hydrogel), the EV-MAP hydrogel comprising one or more EV-HMPs of any one of claims 1 to 3 and 8 to 23 and a crosslinker, wherein at least two EV-HMPs are linked by a crosslinker.

26. The EV-MAP hydrogel of claim 25, wherein at least 50%, at least 60%, at least 70%, at least 80%, at least 85%, or at least 90% of EV-HMPs in the hydrogel are linked by the crosslinker.

27. A method of making an extracellular vesicle microporous annealed particle hydrogel (EV-MAP hydrogel), the method comprising contacting one or more EV-HMPs of any one of claims 1 to 3 and 8 to 23 with a crosslinker to obtain the EV-MAP hydrogel.

28. The method of claim 27, comprising contacting the one or more EV-HMPs with the crosslinker in vivo or in situ.

29. The EV-MAP hydrogel of claim 25 or 26 or the method of claim 27 or 28, wherein the crosslinker comprises two or more azide groups.

30. The EV-HAP hydrogel or method of claim 29, wherein the crosslinker comprises tetra-arm PEG azide.

31. An EV-MAP hydrogel prepared by the method of any one of claims 28 to 30.

32. A pharmaceutical composition comprising one or more EV-HMPs of any one of claims 1 to 3 and 8 to 23 and a pharmaceutically appropriate carrier.

33. A pharmaceutical composition comprising the EV-MAP hydrogel of any one of claims 25 to 26 and 29 to 31 and a pharmaceutically appropriate carrier.

34. A kit comprising: (a) the pharmaceutical composition of claim 32 and (b) a crosslinker.

35. The kit of claim 34, wherein the crosslinker comprises two or more azide groups.

36. The kit of claim 35, wherein the crosslinker comprises tetra-polyethylene glycol-azide.

37. A method of inducing and/or increasing angiogenesis and/or axonogenesis in a subject, the method comprising administering to the subject a therapeutically effective amount of the pharmaceutical composition of claim 32 or 33 to the subject such that angiogenesis and/or axonogenesis is increased in the subject.

38. The method of claim 37 wherein angiogenesis and/or axonogenesis is increased in the brain of the subject.

39. A method of inducing tissue repair in a subject, the method comprising administering to the subject a therapeutically effective amount of the pharmaceutical composition of claim 32 or 33 to the subject such that the tissue repair in the subject is induced.

40. The method of claim 39, wherein the tissue repair comprises brain tissue repair.

41. The method of any one of claims 37 to 40, wherein the subject has or is suspected of having a brain injury.

42. The method of claim 41, wherein the brain injury comprises or is caused by a traumatic injury (e.g., external injury), an aneurysm, a stroke, an infection, a cancer, or a combination of any thereof.

43. A method of treating a stroke in a subject, the method comprising administering to the subject a therapeutically effective amount of the pharmaceutical composition of claim 32 or 33 such that the stroke is treated in the subject.

44. The method of claim 43, wherein treating a stroke comprises reducing or preventing short-term tissue damage at the stroke site.

45. The method of claim 44, wherein the short-term tissue damage comprises damage to surrounding tissue at a stroke site that occurs within 24 hours of the stroke.

46. The method of claim 43, wherein treating a stroke comprises promoting and/or inducing long-term recovery after the stroke.

47. The method of any one of claims 43 to 46, wherein treating the stroke comprises inducing and/or increasing angiogenesis and/or axogenesis at the site of the stroke.

48. The method of any one of claims 42 to 47, wherein the stroke is a hemorrhagic stroke.

49. The method of any one of claims 42 to 47, wherein the stroke is an occlusive stroke.

50. The method of any one of claims 37 to 49, comprising administering to the subject a therapeutically effective amount of the pharmaceutical composition of claim 32.

51. The method of claim 50, further comprises administering a crosslinker to the subject.

52. The method of claim 51, wherein the crosslinker is administered before the pharmaceutical composition.

53. The method of claim 51, wherein the crosslinker is administered simultaneously with pharmaceutical composition.

54. The method of claim 51, wherein the crosslinker is administered after the pharmaceutical composition.

55. The method of claim 54, wherein the crosslinker is administered 1 to 10 days after the pharmaceutical composition.

56. The method of claim 55, wherein the crosslinker is administered 5 days after the pharmaceutical composition.

57. The method of any one of claims 51 to 56, wherein the crosslinker comprises two or more azide groups.

58. The method of claim 57, wherein the crosslinker comprises tetra-arm PEG-azide.

59. The method of any one of claims 37 to 58, wherein the pharmaceutical composition and/or the crosslinker are each delivered into the brain of the subject.

60. The method of any one of claims 37 to 59, wherein the pharmaceutical composition and/or the crosslinker are each administered via stereotaxic injection.

61. The method of any one of claims 37 to 60, wherein the pharmaceutical composition and/or the crosslinker are each administered intrathecally, intraventricularly, via direct infarct injection, via peri-infarct injection, or any combination thereof.

62. The method of any one of claims 42 to 61, wherein the pharmaceutical composition and/or the crosslinker are each administered into a stroke core of the subject.

63. The method of any one of claims 51 to 62, wherein the crosslinker is administered via the same route as the therapeutically effective amount of the pharmaceutical composition.

64. The method of any one of claims 37 to 63, wherein the subject is a mammal.

65. A method of preparing a population of reactive astrocytes, the method comprising culturing a population of astrocytes in a culture medium comprising at least one cytokine or cytokine antibody.

66. The method of claim 65, wherein the medium comprising the at least one cytokine or cytokine antibody comprises IL-4, IL-1α, TNF, C1q, or any combination thereof.

67. The method of claim 66, wherein the medium comprising the at least one cytokine or cytokine antibody comprises (a) IL-1α, TNF, and/or C1q; or (b) IL-4 and/or C1q.

68. A method of preparing a population of activated T-cells, the method comprising culturing a population of T-cells in a culture medium comprising at least one cytokine or cytokine antibody.

69. The method of claim 68, wherein the medium comprising the at least one cytokine or cytokine antibody comprises anti-CD28 antibody, anti-IL4 antibody, IL-2, IL-12, anti-IFNγ antibody, IL-4, TGFβ1, or any combination thereof.

70. The method of claim 69, wherein the medium comprising the at least one cytokine or cytokine antibody comprises (a) anti-CD28 antibody, anti-IL4 antibody, IL-2, and/or IL-12; (b) anti-CD28 antibody, anti-IFNγ antibody, IL-2, and/or IL-4; or (c) anti-CD28 antibody, anti-IFNγ antibody, anti-IL4 antibody, and/or TGFβ1.

71. An extracellular vesicle hydrogel microparticle (EV-HMP), the microparticle comprising: (i) a hydrogel microparticle (HMP) functionalized with a strained alkyne and (ii) an extracellular vesicle (EV) labeled with an azido sugar derived or collected from a cell population, wherein the cell population comprises astrocytes or T-cells cultured in a medium comprising at least one of an anti-CD28 antibody, an anti-IL4 antibody, IL-2, IL-12, an anti-IFNγ antibody, IL-4, TGFβ1, IL-1α, TNF, C1q, or any combination thereof, and wherein the extracellular vesicle is immobilized to the HMP via a stable triazole linkage between the strained alkyne and the azido sugar.


SEQUENCE LISTING (5 sequences)

SEQ ID NO: 1 - protein, 5 aa, synthetic construct
RGDSP

SEQ ID NO: 2 - protein, 10 aa, synthetic construct
RGDSPGERCG

SEQ ID NO: 3 - protein, 14 aa, synthetic construct
NQEQVSPLGGERCG

SEQ ID NO: 4 - protein, 8 aa, synthetic construct
FKGGERCG

SEQ ID NO: 5 - protein, 16 aa, synthetic construct
GCRDGPQGIWGQDRCG